SEQUENCE LISTING (394 sequences)

SEQ ID NO: 1 - protein, 14 aa, Homo sapiens
HVTGASSSSSSSTK

SEQ ID NO: 2 - protein, 13 aa, Homo sapiens
SLDTPDFTDIVLQ

SEQ ID NO: 3 - protein, 14 aa, Homo sapiens
VIIDQLPDLMGLKA

SEQ ID NO: 4 - protein, 12 aa, Artificial Sequence
  Synthetic Construct
  MISC_FEATURE (4)..(4): Xaa = A or S
  MISC_FEATURE (7)..(7): Xaa = N or D
  MISC_FEATURE (9)..(9): Xaa = T or S
  MISC_FEATURE (10)..(10): Xaa = S or L
IEVXNLXGXXRK

SEQ ID NO: 5 - protein, 11 aa, Homo sapiens
  MISC_FEATURE (1)..(1): Xaa = A or D
  MISC_FEATURE (4)..(4): Xaa = E or T
  MISC_FEATURE (6)..(6): Xaa = V or L
XGAXEXLLLAR

SEQ ID NO: 6 - protein, 19 aa, Homo sapiens
  MISC_FEATURE (15)..(15): Xaa = V or L
  MISC_FEATURE (16)..(16): Xaa = D or E
ISLDTPDFTDIVLQXXDIR

SEQ ID NO: 7 - protein, 9 aa, Homo sapiens
  MISC_FEATURE (2)..(2): Xaa = N or A
  MISC_FEATURE (6)..(6): Xaa = S or Q
  MISC_FEATURE (7)..(7): Xaa = E or D
  MISC_FEATURE (9)..(9): Xaa = R or V
AXLDGXXRX

SEQ ID NO: 8 - protein, 18 aa, Homo sapiens
  MISC_FEATURE (9)..(9): Xaa = D or N
ISLETNNNXVAIPLTGVK

SEQ ID NO: 9 - protein, 13 aa, Homo sapiens
  MISC_FEATURE (10)..(10): Xaa = S or T
  MISC_FEATURE (12)..(12): Xaa = N or D
  MISC_FEATURE (13)..(13): Xaa = H or R
EASALDFDVXNXX

SEQ ID NO: 10 - protein, 10 aa, Homo sapiens
  MISC_FEATURE (6)..(6): Xaa = V or I
IYWTDXSLKT

SEQ ID NO: 11 - protein, 8 aa, Homo sapiens
  MISC_FEATURE (2)..(2): Xaa = I or V
  MISC_FEATURE (6)..(6): Xaa = N or V
  MISC_FEATURE (8)..(8): Xaa = N or A
AXVVNXEX

SEQ ID NO: 12 - protein, 12 aa, Homo sapiens
  MISC_FEATURE (5)..(5): Xaa = C or S
RIESXDLSGANR

SEQ ID NO: 13 - protein, 9 aa, Homo sapiens
  MISC_FEATURE (3)..(3): Xaa = S or N
  MISC_FEATURE (7)..(7): Xaa = V or I
  MISC_FEATURE (8)..(8): Xaa = L or G
  MISC_FEATURE (9)..(9): Xaa = I or K
CAXGQCXXX

SEQ ID NO: 14 - protein, 10 aa, Homo sapiens
  MISC_FEATURE (3)..(3): Xaa = S or H
  MISC_FEATURE (4)..(4): Xaa = F or N
  MISC_FEATURE (5)..(5): Xaa = P or V
  MISC_FEATURE (8)..(8): Xaa = I or S
  MISC_FEATURE (10)..(10): Xaa = G or K
CDXXXCDXDX

SEQ ID NO: 15 - protein, 15 aa, Homo sapiens
  MISC_FEATURE (1)..(1): Xaa = N or A
XHVTGASSSSSSSTK

SEQ ID NO: 16 - protein, 12 aa, Homo sapiens
IEVANLNGTSRK

SEQ ID NO: 17 - protein, 11 aa, Homo sapiens
AGAEEVLLLAR

SEQ ID NO: 18 - protein, 19 aa, Homo sapiens
ISLDTPDFTDIVLQVDDIR

SEQ ID NO: 19 - protein, 9 aa, Homo sapiens
ANLDGQERR

SEQ ID NO: 20 - protein, 18 aa, Homo sapiens
ISLETNNNDVAIPLTGVK

SEQ ID NO: 21 - protein, 13 aa, Homo sapiens
EASALDFDVSNNH

SEQ ID NO: 22 - protein, 10 aa, Homo sapiens
IYWTDVSLKT

SEQ ID NO: 23 - protein, 8 aa, Homo sapiens
AIVVNAER

SEQ ID NO: 24 - protein, 12 aa, Homo sapiens
RIESCDLSGANR

SEQ ID NO: 25 - protein, 9 aa, Homo sapiens
CASGQCVLI

SEQ ID NO: 26 - protein, 10 aa, Homo sapiens
CDSFPDCIDG

SEQ ID NO: 27 - protein, 15 aa, Homo sapiens
NHVTGASSSSSSSTK

SEQ ID NO: 28 - protein, 12 aa, Homo sapiens
IEVSNLDGSLRK

SEQ ID NO: 29 - protein, 11 aa, Homo sapiens
DGATELLLLAR

SEQ ID NO: 30 - protein, 19 aa, Homo sapiens
ISLDTPDFTDIVLQLEDIR

SEQ ID NO: 31 - protein, 9 aa, Homo sapiens
AALDGSDRV

SEQ ID NO: 32 - protein, 18 aa, Homo sapiens
ISLETNNNNVAIPLTGVK

SEQ ID NO: 33 - protein, 13 aa, Homo sapiens
EASALDFDVTNDR

SEQ ID NO: 34 - protein, 10 aa, Homo sapiens
IYWTDISLKT

SEQ ID NO: 35 - protein, 8 aa, Homo sapiens
AVVVNPEK

SEQ ID NO: 36 - protein, 12 aa, Homo sapiens
RIESSDLSGANR

SEQ ID NO: 37 - protein, 9 aa, Homo sapiens
CANGQCIGK

SEQ ID NO: 38 - protein, 10 aa, Homo sapiens
CDHNVDCSDK

SEQ ID NO: 39 - protein, 15 aa, Homo sapiens
AHVTGASSSSSSSTK

SEQ ID NO: 40 - protein, 35 aa, Homo sapiens
ERVHKVKASRDVIIDQLPDLMGLKAVNVAKVVGTN

SEQ ID NO: 41 - protein, 35 aa, Homo sapiens
ERVHKRSAEREVIIDQLPDLMGLKATNVHRVIGSN

SEQ ID NO: 42 - protein, 35 aa, Homo sapiens
  MISC_FEATURE (6)..(6): Xaa = V or R
  MISC_FEATURE (7)..(7): Xaa = K or S
  MISC_FEATURE (9)..(9): Xaa = S or E
  MISC_FEATURE (11)..(11): Xaa = D or E
  MISC_FEATURE (26)..(26): Xaa = V or T
  MISC_FEATURE (29)..(29): Xaa = A or H
  MISC_FEATURE (30)..(30): Xaa = K or R
  MISC_FEATURE (32)..(32): Xaa = V or I
  MISC_FEATURE (34)..(34): Xaa = T or S
ERVHKXXAXRXVIIDQLPDLMGLKAXNVXXVXGXN

SEQ ID NO: 43 - protein, 42 aa, Homo sapiens
RISLETNNNDVAIPLTGVKEASALDFDVSNNHIYWTDVSLKT

SEQ ID NO: 44 - protein, 42 aa, Homo sapiens
RISLETNNNNVAIPLTGVKEASALDFDVTDNRIYWTDISLKT

SEQ ID NO: 45 - protein, 42 aa, Homo sapiens
  MISC_FEATURE (10)..(10): Xaa = D or N
  MISC_FEATURE (29)..(29): Xaa = S or T
  MISC_FEATURE (30)..(30): Xaa = N or D
  MISC_FEATURE (32)..(32): Xaa = H or R
  MISC_FEATURE (38)..(38): Xaa = V or I
RISLETNNNXVAIPLTGVKEASALDFDVXXNXIYWTDXSLKT

SEQ ID NO: 46 - protein, 138 aa, Homo sapiens
KGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDEL

SEQ ID NO: 47 - protein, 138 aa, Homo sapiens
KGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDEL

SEQ ID NO: 48 - protein, 138 aa, Homo sapiens
  MISC_FEATURE (6)..(6): Xaa = P or T
  MISC_FEATURE (12)..(12): Xaa = V or M
  MISC_FEATURE (19)..(19): Xaa = N or D
  MISC_FEATURE (20)..(20): Xaa = L or E
  MISC_FEATURE (22)..(22): Xaa = T or S
  MISC_FEATURE (32)..(32): Xaa = D or Q
  MISC_FEATURE (35)..(35): Xaa = A or T
  MISC_FEATURE (37)..(37): Xaa = A or F
  MISC_FEATURE (46)..(46): Xaa = G or V
  MISC_FEATURE (54)..(54): Xaa = P or T
  MISC_FEATURE (57)..(57): Xaa = D or E
  MISC_FEATURE (59)..(59): Xaa = Q or H
  MISC_FEATURE (63)..(63): Xaa = E or L
  MISC_FEATURE (64)..(64): Xaa = G or N
  MISC_FEATURE (70)..(70): Xaa = A or E
  MISC_FEATURE (71)..(71): Xaa = A or S
  MISC_FEATURE (74)..(74): Xaa = P or Q
  MISC_FEATURE (77)..(77): Xaa = R or S
  MISC_FEATURE (81)..(81): Xaa = V or I
  MISC_FEATURE (83)..(83): Xaa = L or G
  MISC_FEATURE (84)..(84): Xaa = R or A
  MISC_FEATURE (88)..(88): Xaa = D or N
  MISC_FEATURE (90)..(90): Xaa = E or D
  MISC_FEATURE (92)..(92): Xaa = D or N
  MISC_FEATURE (96)..(96): Xaa = R or K
  MISC_FEATURE (100)..(100): Xaa = A or K
  MISC_FEATURE (101)..(101): Xaa = D or N
  MISC_FEATURE (103)..(103): Xaa = A or V
  MISC_FEATURE (104)..(104): Xaa = I or L
  MISC_FEATURE (108)..(108): Xaa = P or I
  MISC_FEATURE (109)..(109): Xaa = N or D
  MISC_FEATURE (115)..(115): Xaa = S or N
  MISC_FEATURE (119)..(119): Xaa = V or I
  MISC_FEATURE (120)..(120): Xaa = L or G
  MISC_FEATURE (121)..(121): Xaa = I or K
  MISC_FEATURE (122)..(122): Xaa = K or H
  MISC_FEATURE (123)..(124): Xaa = Q or K
  MISC_FEATURE (127)..(127): Xaa = S or H
  MISC_FEATURE (128)..(128): Xaa = F or N
  MISC_FEATURE (129)..(129): Xaa = P or V
  MISC_FEATURE (132)..(132): Xaa = I or S
  MISC_FEATURE (134)..(134): Xaa = G or K
KGDGTXRCSCPXHLVLLQXXLXCGEPPTCSPXQFXCXTGEIDCIPXAWRCDGFXECXDXSDEXXCPVCSXXQFXCAXGQCXDXXLRCXGXAXCQDXSDEXXCXXLCLXXQFRCAXGQCXXXXXXCDXXXDCXDXSDEL

SEQ ID NO: 49 - protein, 1905 aa, Mus musculus
MRRWWGALLLGALLCAHGIASSLECACGRSHFTCAVSALGECTCIPAQWQCDGDNDCGDHSDEDGCTLPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEESCPSAVPSPPCNLEEFQCAYGRCILDIYHCDGDDDCGDWSDESDCSSHQPCRSGEFMCDSGLCINSGWRCDGDADCDDQSDERNCTTSMCTAEQFRCRSGRCVRLSWRCDGEDDCADNSDEENCENTGSPQCASDQFLCWNGRCIGQRKLCNGINDCGDSSDESPQQNCRPRTGEENCNVNNGGCAQKCQMVRGAVQCTCHTGYRLTEDGRTCQDVNECAEEGYCSQGCTNTEGAFQCWCEAGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQSLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKINSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASSRQVIISSNLTWPNGLAIDYGSQRLYWADAGMKTIEFAGLDGSKRKVLIGSQLPHPFGLTLYGQRIYWTDWQTKSIQSADRLTGLDRETLQENLENLMDIHVFHRQRPPVTTLCAVENGGCSHLCLRSPNPSGFSCTCPTGINLLRDGKTCSPGMNSFLIFARRIDVRMVSLDIPYFADVVVPINMTMKNTIAIGVDPLEGKVYWSDSTLHRISRASLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRTVLINNNLGWPNGLTVDKTSSQLLWADAHTERIEVADLNGANRHTLVSPVQHPYGLTLLDSYIYWTDWQTRSIHRADKSTGSNVILVRSNLPGLMDIQAVDRAQPLGFNKCGSRNGGCSHLCLPRPSGFSCACPTGIQLKGDRKTCDPSPETYLLFSSRGSIRRISLDTDDHTDVHVPVPGLNNVISLDYDSVHGKVYYTDVFLDVIRRADLNGSNMETVIGHGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVSHVSHPFALTQQDRWIYWTDWQTKSIQRVDKYSGRNKETVLANVEGLMDIIVVSPQRQTGTNACGVNNGGCTHLCFARASDFVCACPDEPDGHPCSLVPGLVPPAPRATSMNEKSPVLPNTLPTTLHSSTTKTRTSLEGAGGRCSERDAQLGLCAHSNEAVPAAPGEGLHVSYAIGGLLSILLILLVIAALMLYRHRKSKFTDPGMGNLTYSNPSYRTSTQEVKLEAAPKPAVYNQLCYKKEGGPDHSYTKEKIKIVEGIRLLAGDDAEWGDLKQLRSSRGGLLRDHVCMKTDTVSIQASSGSLDDTETEQLLQEEQSECSSVHTAATPERRGSLPDTGWKHERKLSSESQV

SEQ ID NO: 50 - protein, 1905 aa, Homo sapiens
MRRQWGALLLGALLCAHGLASSPECACGRSHFTCAVSALGECTCIPAQWQCDGDNDCGDHSDEDGCILPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEENCPSAVPAPPCNLEEFQCAYGRCILDIYHCDGDDDCGDWSDESDCSSHQPCRSGEFMCDSGLCINAGWRCDGDADCDDQSDERNCTTSMCTAEQFRCHSGRCVRLSWRCDGEDDCADNSDEENCENTGSPQCALDQFLCWNGRCIGQRKLCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAIGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVSHVSHPFALTQQDRWIYWTDWQTKSIQRVDKYSGRNKETVLANVEGLMDIIVVSPQRQTGTNACGVNNGGCTHLCFARASDFVCACPDEPDSRPCSLVPGLVPPAPRATGMSEKSPVLPNTPPTTLYSSTTRTRTSLEEVEGRCSERDARLGLCARSNDAVPAAPGEGLHISYAIGGLLSILLILVVIAALMLYRHKKSKFTDPGMGNLTYSNPSYRTSTQEVKIEAIPKPAMYNQLCYKKEGGPDHNYTKEKIKIVEGICLLSGDDAEWDDLKQLRSSRGGLLRDHVCMKTDTVSIQASSGSLDDTETEQLLQEEQSECSSVHTAATPERRGSLPDTGWKHERKLSSESQV

SEQ ID NO: 51 - protein, 1905 aa, Rattus norvegicus
MRRWWGALLLGALLCAHGTASNLECACGRSHFTCAVSALGECTCIPAQWQCDGDNDCGDHSDEDGCTLPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEESCPSAVPSPPCNLEEFQCAYGRCILDIYHCDGDDDCGDWSDESDCSSHQPCRSGEFMCDSGLCVNAGWRCDGDADCDDQSDERNCTTSMCTAEQFRCRSGRCVRLSWRCDGEDDCADNSDEENCENTGSPQCASDQFLCWNGRCIGQRKLCNGVNDCGDNSDESPQQNCRPRTGEENCNVNNGGCAQKCQMIRGAVQCTCHTGYRLTEDGRTCQDVNECAEEGYCSQGCTNSEGAFQCWCEAGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQSLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKINSHACAQSLDKFLLFARRMDIRRISFDTEDLSDDVIPLADVRSAVALDWDSRDDHVYWTDVSTDTISRAKWDGTGQKVVVDTSLESPAGLAIDWVTNKLYWTDAGTDRIEVANTDGSMRTVLIWENLDRPRDIVVEPMGGYMYWTDWGASPKIERAGMDASNRQVIISSNLTWPNGLAIDYGSQRLYWADAGMKTIEFAGLDGSKRKVLIGSQLPHPFGLTLYGQRIYWTDWQTKSIQSADRLTGLDRETLQENLENLMDIHVFHRQRPPVTTPCAVENGGCSHLCLRSPSPSGFSCTCPTGINLLLDGKTCSPGMNSFLIFARRIDVRMVSLDIPYFADVVVPINMTMKNTIAIGVDPLEGKVYWSDSTLHRISRASLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRTVLINNNLGWPNGLTVDKTSSQLLWADAHTERIEVADLNGANRHTLVSPVQHPYGLTLLDSYIYWTDWQTRSIHRADKSTGSNVILVRSNLPGLMDIQAVDRAQPLGFNKCGSRNGGCSHLCLPRPSGFSCACPTGIQLKGDGKTCDPSPETYLLFSSRGSIRRISLDTDDHTDVHVPVPGLNNVISLDYDSVDGKVYYTDVFLDVIRRADLNGSNMETVIGHGLKTTDGLAVDWVARNLYWTDTGRNTIEASRLDGSCRKVLINNSLDEPRAIAVFPRKGYLFWTDWGHIAKIERANLDGSERKVLINADLGWPNGLTLDYDTRRIYWVDAHLDRIESADLNGKLRQVLVSHVSHPFALTQQDRWIYWTDWQTKSIQRVDKYSGRNKETVLANVEGLMDIIVVSPQRQTGTNACGVNNGGCSHLCFARASDFVCACPDEPDSHPCSLVPGLMPPAPRATSLNEKSPVLPNTLPTTLHSSTTRTRTSPEGAEGRCSERDAQLGLCAHSNEAVPAAPGEGLHVSYAVGGLLSVLLILLVTAALMLYRHRKSKFTDPGMGNLTYSNPSYRTSTQEVKIEAAPKPAMYNQLCYKKEGGPDHSYTKEKIKIVEGIHLLAGHDAEWGDLKQLRSSRGGLLRDHVCMKTDTVSIQASSGSLDDTETEQLLQEEQSECSSVHTATTPERRGSLPDTGWKHERKLSSESQV

SEQ ID NO: 52 - protein, 1944 aa, Gallus gallus
MWLMEDLTVTRSPLELGLCQHSLHLHAEGVLNGADPTLGSKLETKGFGRKISRLAKRQPHKGITSSTECSCGRNHFTCAVSAFGECTCIPAQWQCDGDNDCGDHSDEDGCMLPTCSPLDFHCDNGKCIRRSWVCDGDNDCEDDSDEQDCPPRECEEDEFSCQNGYCIRSLWHCDGDNDCGDNSDEQCDMRKCSEKEFRCSDGSCIAEHWFCDGDTDCKDGSDEENCPSDVPAATCSLEEFQCAYGRCILDIYHCDGDDDCGDWSDESDCSSHQPCRSGEFMCNSGLCINAGWRCDGDSDCDDQSDERNCTTSMCTADQFRCKSGRCVRLSWRCDGEDDCSDNSDEENCENTGTPQCAPDQFLCGNGRCIGQRKLCNGANDCGDGSDESPHQNCRPRTGEENCNVNNGGCAQKCQMVRGMVQCTCHTGYRLLEDGRSCQDVNECAEEGYCSQGCTNSEGGFQCWCEQGYELRPDKRSCKALGPEPVLLFANRIDIRQVLPHRSEYTLLLNNLENAIALDFHHSKELVFWSDVTLDRIMRANLNGSNVEEVVSTGLESPGGLAIDWIHDKLYWTDSGTSRIEVANLDGTHRKVLLWQNLEKPRAIALHPMEGTIYWTDWGNTPRIEYSNMDGSNRRIIADTHLFWPNGLTIDYAGHRMYWVDAKHHVIERADLDGRNRKAVISQGLPHPFAITVFEDSLYWTDWHTKSINSANKFTGKNQEIIRNKLHFPMDIHTLHPQRQPAGRNRCGANNGGCTHLCLPSSKDYTCACPTGFRKTSSHACAQSLDKFLLFARRMDIRRISFDTDDLSDDVIPLADVRSAVALDWDSKDDYVYWTDVSTDSISRAKWDGSGQEVVVDTSLESPAGLAIDWVTNKLYWTDAGTDRIEVSNTDGTMRTVLIWENLDRPRDIVVDPVGGFMYWTDWGANPKIERAGMDASNRLVIISSNLTWPNGLAIDYESQRLYWADAGMKTIEYASLDGSNRKVLIGSNLPHPFGLTLYGERIYWTDWQAKSIQSADRRTGQARETLQDNLENLMDIHVFHRHRPPVRTPCEVNNGGCSHLCLLAPLPKGYSCTCPTGINLQSDGKTCSTGMTSFLIFARRTDIRMVSLDIPYFADVVVSVNVTMKNTIAIGVDPHEGKVYWSDSTLRKISRAALDGSKFEDIITTGLLTTDGLAVDAIGRKIYWTDTGTNRIEVGNLDGSMRKVLVWQNLDSPRAIALYHEMGYMYWTDWGENAKLERSGMDGSGRVVLISNNLGWPNGLAVDKAGSQLLWADAHTERIEAADLNGANRRTLLSPVQHPYGLTLLDSYIYWTDWQTRSIHRADKDSGANVILVRANLPGLMDIQAVDRARPLGFNKCGVRNGGCSHLCLPHPTGFSCACPTGIQLKRDEQTCDSSPETYLLFSSRASIRRISLDTSDHTDVHIPVPELNNVISLDYDSVDGKIYYTDVFLDVIRRSDLNGSNMETVIGQGLKTTDGLAVDWVARNLYWTDTGRNTVEVARLDGSSRKVLINNSLDEPRAIAVFPKKGYLFWTDWGHIAKIERANLDGSERKILINTDLGWPNGLTLDYDTRRIYWVDAHLDRIESCDLNGKLRQVLVSQVSHPFALTQQDRWIYWTDWQTKSIQRVDKYSGRNKETVLANVEGLMDIIVVSPQRQTGTNACGVNNGGCTHLCFARASDFVCACPDEPDGRPCSTVPGVVPFGPEITSERSQTPPGRIGTSTTKPLTSLEEEEGNCSDKDARRGLCTHANEAVLATMGEGLHVSYIIGGLLSVLFILLLIASLIIYRHNKSKFTDPGLGNLTYSNPSYRTSTQEVKIETIPKPTMYNQLCYKKETGPDHSYTKEKIKIVEGICLLSSDDSEWDDLKQIRSSRGGILRDHVCMKTDTVSIQASSGSLDDTETEQLLQEEQSECSSVNTAAATPERRGSLPDTGWKHQRKPSTESEV

SEQ ID NO: 53 - protein, 1614 aa, Mus musculus
METAPTRAPPPPPPPLLLLVLYCSLVPAAASPLLLFANRRDVRLVDAGGVKLESTIVASGLEDAAAVDFQFSKGAVYWTDVSEEAIKQTYLNQTGAAAQNIVISGLVSPDGLACDWVGKKLYWTDSETNRIEVANLNGTSRKVLFWQDLDQPRAIALDPAHGYMYWTDWGEAPRIERAGMDGSTRKIIVDSDIYWPNGLTIDLEEQKLYWADAKLSFIHRANLDGSFRQKVVEGSLTHPFALTLSGDTLYWTDWQTRSIHACNKWTGEQRKEILSALYSPMDIQVLSQERQPPFHTPCEEDNGGCSHLCLLSPREPFYSCACPTGVQLQDNGKTCKTGAEEVLLLARRTDLRRISLDTPDFTDIVLQVGDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIVLHPVMGLMYWTDWGENPKIECANLDGRDRHVLVNTSLGWPNGLALDLQEGKLYWGDAKTDKIEVINIDGTKRKTLLEDKLPHIFGFTLLGDFIYWTDWQRRSIERVHKVKASRDVIIDQLPDLMGLKAVNVAKVVGTNPCADGNGGCSHLCFFTPRATKCGCPIGLELLSDMKTCIIPEAFLVFTSRATIHRISLETNNNDVAIPLTGVKEASALDFDVSNNHIYWTDVSLKTISRAFMNGSSVEHVIEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERMVIADDLPYPFGLTQYSDYIYWTDWNLHSIERADKTSGRNRTLIQGHLDFVMDILVFHSSRQDGLNDCVHSNGQCGQLCLAIPGGHRCGCASHYTLDPSSRNCSPPSTFLLFSQKFAISRMIPDDQLSPDLVLPLHGLRNVKAINYDPLDKFIYWVDGRQNIKRAKDDGTQPSMLTSPSQSLSPDRQPHDLSIDIYSRTLFWTCEATNTINVHRLDGDAMGVVLRGDRDKPRAIAVNAERGYMYFTNMQDHAAKIERASLDGTEREVLFTTGLIRPVALVVDNALGKLFWVDADLKRIESCDLSGANRLTLEDANIVQPVGLTVLGRHLYWIDRQQQMIERVEKTTGDKRTRVQGRVTHLTGIHAVEEVSLEEFSAHPCARDNGGCSHICIAKGDGTPRCSCPVHLVLLQNLLTCGEPPTCSPDQFACTTGEIDCIPGAWRCDGFPECADQSDEEGCPVCSASQFPCARGQCVDLRLRCDGEADCQDRSDEANCDAVCLPNQFRCTSGQCVLIKQQCDSFPDCADGSDELMCEINKPPSDDIPAHSSAIGPVIGIILSLFVMGGVYFVCQRVMCQRYTGASGPFPHEYVGGAPHVPLNFIAPGGSQHGPFPGIPCSKSVMSSMSLVGGRGSVPLYDRNHVTGASSSSSSSTKATLYPPILNPPPSPATDPSLYNVDVFYSSGIPATARPYRPYVIRGMAPPTTPCSTDVCDSDYSTSRWKSSKYYLDLNSDSDPYPPPPTPHSQYLSAEDSCPPSPGTERSYCHLFPPPPSPCTDSS

SEQ ID NO: 54 - protein, 1615 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 55 - protein, 1615 aa, Rattus norvegicus
METAPTRAPPPPPQPLLLLLALCCSLVPAAASPLLLFANRRDVRLVDAGGVKLESTIVASGLEDAAAVDFQFSKGAVYWTDVSEEAIKQTYLNQTGAAAQNIVISGLVSPDGLACDWVGKKLYWTDSETNRIEVANLNGTSRKVLFWQDLDQPRAIALDPAHGYMYWTDWGEAPRIERAGMDGSTRKIIVDSDIYWPNGLTIDLEEQKLYWADAKLSFIHRANLDGSFRQKVVEGSLTHPFALTLSGDTLYWTDWQTRSIHACNKWTGEKRKEILSALYSPMDIQVLSQERQPHFHTPCEEGNGGCSHLCLLSPREPFYSCACPTGVQLQDNGKTCKAGAEEVLLLARRTDLRRISLDTPDFTDIVLQVGDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIVLHPVMGLMYWTDWGENPKIECANLDGRDRHVLVNTSLGWPNGLALDLQEGKLYWGDAKTDKIEVINIDGTKRQTLLEDKLPHIFGFTLLGDFIYWTDWQRRSIERVHKVKASRDVIIDQLPDLMGLKAVNVAKVVGTNPCADGNGGCSHLCFFTPHATKCGCPIGLELLSDMKTCIIPEAFLVFTSRATIHRISLETNNNDVAIPLTGVKEASALDFDVSNNHIYWTDVSLKTISRAFMNGSSVEHVIEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERMVIADDLPYPFGLTQYSDYIYWTDWNLHSIERADKTSGRNRTLIQGHLDFVMDILVFHSSRQDGLNDCVHSNGQCGQLCLAIPGGHRCGCASHYTLDPSSRNCSPPSTFLLFSQKFAISRMIPDDQLSPDLILPLHGLRNVKAINYDPLDKFIYWVDGRQNIKRAKDDGTQPSMLTSPSQSLSPDRQPHDLSIDIYSRTLFWTCEATNTINVHRLDGDAMGVVLRGDRDRPRAIAVNAERGYMYFTNMQDHAAKIERASLDGTEREVLFTTGLIRPVALVVDNALGKLFWVDADLKRIESCDLSGANRLTLEDANIVQPVGLTVLGRHLYWIDRQQQMIERVEKTTGDKRTRVQGRVTHLTGIHAVEEVSLEEFSAHPCARDNGGCSHICIAKGDGTPRCSCPVHLVLLQNLLTCGEPPTCSPDQFACATGEIDCIPGAWRCDGFPECADQSDEEGCPVCSASQFPCARGQCVDLRLRCDGEADCQDGSDEANCDAVCLPNQFRCASGQCVLIKQQCDSFPDCADGSDELMCEINKPPSDDVPAHSSAIGPVIGIILSLFVMGGVYFVCQRVMCQRYTGASGPFPHEYVGGTPHVPLNFIAPGGSQHGPFPGIPCSKSMMSSVSLVGGRGSVPLYDRNHVTGASSSSSSSTKATLYPPILNPPPSPATDPSLYNMDMFYSSNIPSTARPYRPYIIRGMAPPTTPCSTDVCDSDYSTSRWKTSKYYLDLNSDSDPYPPPPTPHSQYLSAEDSCPPSPVTERSYCHLFPPPPSPCTDSS

SEQ ID NO: 56 - protein, 1616 aa, Gallus gallus
MEAAPPLPLLLLLRLLAASVLRCEPGSAGASPFLLFANRRDVRLVDAGGTKLESTVVVSGLEDAAAVDFQYSQGIVFWTDVSEEAIKQTYINQTGNVVQNVIISGLVSPDGLACDWIGKKLYWTDSETNRIEVANLNGTSRKVLFWQDLDQPRAIALDPAHGYMYWTDWGETPRIERAGMDSSTRKIIVDSDIYWPNGLTIDLDEQKLYWADAKLSFIHRANLDGSFRQKVVEGSLTHPFALTLSGDTLYWTDWQTRSIHACNKRTGEKRREILSALYSPMDIQVLSPDRQPYFHTPCEENNGGCSHLCLLSPRDPFYSCACPTGVQLEDDGRTCKSGAEEVLLLARRTDLRRISLDMPDFTDIILQIDNIRHAIAIDYDPVEGYIYWTDDDVRAIRRAYLDGSGAQTLVTTEINHPDGIAVDWVARNLYWTDTGTDRIEVTRLNGTSRKILISENLDEPRAIVLNPVMGYMYWTDWGESPKIECAYLDGSERRVLVNTSLGWPNGLALDLEEDKLYWGDAKTDKIEVINVDGTMRKTLLEDKLPHIFGFTLLGDYIYWTDWQRRSIERVHKIRASRDIIIDQLPDLMGLKATSVTKVFGTNPCAENNGGCSHLCFFTPQETRCACPIGLELLSDMKTCIIPEAFLVFTSRAAIHRISLETNNNDVAIPLTGVKEASALDFDVSDNRIYWTDVSLKTISRAFMNGSSVEHVIEFGLDYPEGMAVDWMGKNLYWADTGTNRIEVARLDGQYRQVLVWKDLDNPRSLALDPTKGYMYWTEWGGKPRIVRAYMDGTNSITLVDKVGRANDLTIDYADQRLYWTDLDTSMIESSNMLGQEREIIADDLPHPFGLTQYSDYIYWTDWNLHSIERADKTSGKNRTLIQGHLDFVMDILVFHSSRQDGLNDCVQNNGHCGHLCLAIPNGFRCGCAAHYTLDPNSRNCSSPTSFLLFSQKSAISRMIPDDQQSPDIILPMHGLRNVKAIDYDPLDKLIYWVDGRQNIIKRAKDDGTQPFTVMSSPNQSQNPEKQPHDLSIDIYSHTLYWTCEATNSVNVHRLNGESIGMVLRGDHDKPRAIVVNAERGYMYFTNMQERAPKIERAALDGTEREVLFTTGLIRPVALVIDNKLGKLFWVDADLKRIESCDLSGANRVTLEDSNILQPMGLTVLGNHLYWIDRQQQMIERVEKTNGYKRTRIQGRIAHLTGIHAVEELDMEEFSAHPCSRDNGGCSHICIAKGDGTPRCSCPEHLVLLQNLLTCGEPPTCSPDQFTCATGEIDCIPMAWRCDGFPECDDQSDEDSCPICSASQFQCEKGQCIDAHLRCNGEIDCQDKSDEVDCDTICLLNQFRCASGQCILLKQQCDSFPDCIDGSDELMCEKSKPSSDEPQPHSSAIGPVIGIILSLFVMGGMYFVCQRVVCQRYAGPNSPFPHEYVSGTPHVPLNFIAPGSSQHGTFTGISCGKSMISSMSLMGGSSGAPLYDRNHVTGASSSSSSSTKATFYPQILNPPPSPATDRSLYNAEMFYSSNIPSTTRSYRPYLIRGTAPPTTPCSTDVCDSDYTTSRWKANKYYIDLNSDSDPYPPPPTPRSQYMSAEESCPPSPATERSYFHLYPPPPSPCTDSS

SEQ ID NO: 57 - protein, 1613 aa, Mus musculus
MGAVLRSLLACSFCVLLRAAPLLLYANRRDLRLVDATNGKENATIVVGGLEDAAAVDFVFGHGLIYWSDVSEEAIKRTEFNKSESVQNVVVSGLLSPDGLACDWLGEKLYWTDSETNRIEVSNLDGSLRKVLFWQELDQPRAIALDPSSGFMYWTDWGEVPKIERAGMDGSSRFVIINTEIYWPNGLTLDYQERKLYWADAKLNFIHKSNLDGTNRQAVVKGSLPHPFALTLFEDTLYWTDWNTHSILACNKYTGEGLREIHSNIFSPMDIHAFSQQRQPNATNPCGIDNGGCSHLCLMSPVKPFYQCACPTGVKLMENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGTIYWGDAKTDKIEVMNTDGTGRRVLVEDKIPHIFGFTLLGDYVYWTDWQRRSIERVHKRSAEREVIIDQLPDLMGLKATSVHRVIGSNPCAEDNGGCSHLCLYRPQGLRCACPIGFELIGDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSDMLGLNREVIADDLPHPFGLTQYQDYIYWTDWSRRSIERANKTSGQNRTIIQGHLDYVMDILVFHSSRQAGWNECASSNGHCSHLCLAVPVGGFVCGCPAHYSLNADNRTCSAPSTFLLFSQKSAINRMVIDEQQSPDIILPIHSLRNVRAIDYDPLDKQLYWIDSRQNSIRKAHEDGGQGFNVVANSVANQNLEIQPYDLSIDIYSRYIYWTCEATNVIDVTRLDGRSVGVVLKGEQDRPRAIVVNPEKGYMYFTNLQERSPKIERAALDGTEREVLFFSGLSKPIALALDSKLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGDIDCIPVAWRCDGFTECEDHSDELNCPVCSESQFQCASGQCIDGALRCNGDANCQDKSDEKNCEVLCLIDQFRCANGQCVGKHKKCDHSVDCSDRSDELDCYPTEEPAPQATNTVGSVIGVIVTIFVSGTIYFICQRMLCPRMKGDGETMTNDYVVHSPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDVNYDSEPVPPPPTPRSQYLSAEENYESCPPSPYTERSYSHHLYPPPPSPCTDSS

SEQ ID NO: 58 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 59 - protein, 1234 aa, Rattus norvegicus
MYWTDWGEVPKIERAGMDGSSRFVIINTEIYWPNGLTLDYEERKLYWADAKLNFIHKSNLDGTNRQAVVKGSLPHPFALTLFEDTLYWTDWNTHSILACNKYTGEGLREIHSNIFSPMDIHAFSQQRQPNATNPCGIDNGGCSHLCLMSPVKPFYQCACPTGVKLLESGKTCKDGATELLLLARRTDLRRISLDTPDFTDIVLQLEDIRHAIAIDYDPVEGYVYWTDDEVRAIRRSFIDGSGSQFVVTAQIAHPDGIAVDWVARNLYWTDTGTDRIEVTRLNGTMRKILISEDLEEPRAIVLDPMAGYMYWTDWGEIPKIERAALDGSDRVVLVNTSLGWPNGLALDYDEGTIYWGDAKTDKIEVMNTDGTGRRVLVEDKIPHIFGFTLLGDYVYWTDWQRRSIERVHKRSAEREVIIDQLPDLMGLKATSVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQAGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGGQGFTVVVSSVPNQNLEIQPYDLSIDIYSRYIYWTCEATNVIDVTRLDGRSVGVVLKGEQDRPRAIVVNPEKGYMYFTNLQERSPKIERAALDGTEREVLFFSGLSKPVALALDSKLSRLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGDIDCIPVAWRCDGFTECEDHSDELNCPVCSESQFQCASGQCIDGALRCNGDANCQDKSDEKNCEVLCLIDQFRCANGQCVGKHKKCDHNVDCSDRSDELDCYFEPTTILQAVQLPALCTAHYTLQH

SEQ ID NO: 60 - protein, 1625 aa, Gallus gallus
  misc_feature (683)..(683): Xaa can be any naturally occurring amino acid
MMQIPPCFKRQKEKCDNFQNPGTERYCVLLRSAPLLLYANRRDLRLVDAANGKENATVIVGGLEDAAAVDFVFVKGLIYWSDVSEEAIKRTEFNKSGSVQNVVISGLLSPDGLACDWLGEKLYWTDSETNRIEVSNLDGSLRKVLFWQELDQPRAIALDPARGFMYWTDWGEVPKIERAGMDGSSRSIIVNTDIYWPNGLTLDYEEQKLYWADAKLNFIHKSNLDGSHRQAVVKGSLPHPFALTLFGDTLYWTDWNTHSILACSKYSGEDLREIHSNIFSPMDIHAFSQKRQPNATNPCGINNGGCSHLCLMSPTKPSYQCACPTGVKLLENGKTCKDGATELLLLARRTDLRRISLDTPDFTDIVLPLEDIRHAIAIDFDPLEGYIYWTDDEVRAIRRSFVDGSGSQFVVTAQIAHPDGIAVDWVARNLYWTDTGTDRIEVTRLNGTMRKILISEDLEEPRAIVLDPMVGYMYWTDWGEIPKIERAALDGSDRIVLVNTSLGWPNGLALDYAESKIYWGDAKTDKIEVMNADGTGRRVLVEDKLPHIFGFTLLGDYVYWTDWQRRSIERVHKRTAEREIIIDQLPDLMGLKATNVHQIIGTNPCAEDNGGCSHLCLYRPQGLRCACPIGLELINDMKTCIVPEAFLLFSRRADIRRISLETNNNNVAIPLTGVKEASALDFXCDRQSNYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRASMDGSERTTLVPNVGRANGLTIDYAKRRLYWTDLDTNLIESSNMLGLDREIIADDLPHPFGLTQYQDYIYWTDWSRRSIERANKTSGQNRTIIQGHLDYVMDILVFHSSRQAGWNECASSNGHCSHLCLAVPVGGFVCGCPAHYSLNSDNRTCSAPTTFLLFSQKNAINRMVIDEQQSPDIILPIHSLRNVRAIDYDPLDKQLYWIDSRQNIIRKAQEDGSQSLTVVISPVPNQNLDMQPYDLSIDIYSRYIYWTCEATNVINVTRLDGRPMGVVLKGDQDRPRAIVVNPEKGYMYFTNLQERSPKIERAALDGTEREVLFFSGLSKPIALAIDSQLGKLFWADSDLRRIESSDLSGANRVVLEDSNILQPVGLTVFENWLYWIDRQQQMIEKIDMTGREGRTKVQARIAQLSDIHAVKELNVQEYRQHPCSQDNGGCSHICIVKGDGTTRCSCPVHLVLLQDELSCGEPPTCSPQQFTCFTGEIDCIPVAWRCDGFTECEDHSDEKNCPVCSDTQFQCESGQCIDSALRCNGEANCQDNSDEKNCEVLCLTSQFRCASGQCIGKSKKCDHNLDCSDSSDEQGCYTTEEPAPQPNNTIGSIIGVILTLFVVGAMYFICQRVLCPRMKGDGETMTNDYVVHGPASVPLGYVPHPSSLSGSLPGMSRGKSVISSLSIMGGSSGPPYDRAHVTGASSSSSSSTKGTYFPPILNPPPSPATERSHYTMEFGYSSNSPSTHRSYSYRPYSYRHFAPPTTPCSTDVCDSDYAPSRRVTATMAKGYTSDLNYDSEPVPPPPTPRSQYLSAEENYESCPPSPYTERSYSHHLYPPPPSPCTDSS

SEQ ID NO: 61 - protein, 206 aa, Mus musculus
MLPPAIHLSLIPLLCILMRNCLAFKNDATEILYSHVVKPVPAHPSSNSTLNQARNGGRHFSSTGLDRNSRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESVSPAKPAQHHRERKRASKSSKHSLS

SEQ ID NO: 62 - protein, 206 aa, Homo sapiens
MLPPAIHFYLLPLACILMKSCLAFKNDATEILYSHVVKPVPAHPSSNSTLNQARNGGRHFSNTGLDRNTRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESMSPAKPVQHHRERKRASKSSKHSMS

SEQ ID NO: 63 - protein, 206 aa, Rattus norvegicus
MLPPAIHLSLIPLLCILMKNCLAFKNDATEILYSHVVKPVSAHPSSNSTLNQARNGGRHFSSTGLDRNSRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESVSPAKPAQHHRERKRASKSSKHSLS

SEQ ID NO: 64 - protein, 206 aa, Gallus gallus
MLLSAIHFYGLLLACTFTRSYSAFKNDATEILYSHVVKPAPASPSSNSTLNQARNGGRHYAGTGSDRNNRVQVGCRELRSTKYISDGQCTSINPLKELVCAGECLPLPLLPNWIGGGYGTKYWSRRSSQEWRCVNDKTRTQRIQLQCQDGSIRTYKITVVTACKCKRYTRQHNESSHNFEGTSQAKPVQHHKERKRASKSSKHSTS

SEQ ID NO: 65 - protein, 20 aa, Homo sapiens
KGDGTPRCSCPVHLVLLQNL

SEQ ID NO: 66 - protein, 20 aa, Homo sapiens
GDGTPRCSCPVHLVLLQNLL

SEQ ID NO: 67 - protein, 20 aa, Homo sapiens
DGTPRCSCPVHLVLLQNLLT

SEQ ID NO: 68 - protein, 20 aa, Homo sapiens
GTPRCSCPVHLVLLQNLLTC

SEQ ID NO: 69 - protein, 20 aa, Homo sapiens
TPRCSCPVHLVLLQNLLTCG

SEQ ID NO: 70 - protein, 20 aa, Homo sapiens
PRCSCPVHLVLLQNLLTCGE

SEQ ID NO: 71 - protein, 20 aa, Homo sapiens
RCSCPVHLVLLQNLLTCGEP

SEQ ID NO: 72 - protein, 20 aa, Homo sapiens
CSCPVHLVLLQNLLTCGEPP

SEQ ID NO: 73 - protein, 20 aa, Homo sapiens
SCPVHLVLLQNLLTCGEPPT

SEQ ID NO: 74 - protein, 20 aa, Homo sapiens
CPVHLVLLQNLLTCGEPPTC

SEQ ID NO: 75 - protein, 20 aa, Homo sapiens
PVHLVLLQNLLTCGEPPTCS

SEQ ID NO: 76 - protein, 20 aa, Homo sapiens
VHLVLLQNLLTCGEPPTCSP

SEQ ID NO: 77 - protein, 20 aa, Homo sapiens
HLVLLQNLLTCGEPPTCSPD

SEQ ID NO: 78 - protein, 20 aa, Homo sapiens
LVLLQNLLTCGEPPTCSPDQ

SEQ ID NO: 79 - protein, 20 aa, Homo sapiens
VLLQNLLTCGEPPTCSPDQF

SEQ ID NO: 80 - protein, 20 aa, Homo sapiens
LLQNLLTCGEPPTCSPDQFA

SEQ ID NO: 81 - protein, 20 aa, Homo sapiens
LQNLLTCGEPPTCSPDQFAC

SEQ ID NO: 82 - protein, 20 aa, Homo sapiens
QNLLTCGEPPTCSPDQFACA

SEQ ID NO: 83 - protein, 20 aa, Homo sapiens
NLLTCGEPPTCSPDQFACAT

SEQ ID NO: 84 - protein, 20 aa, Homo sapiens
LLTCGEPPTCSPDQFACATG

SEQ ID NO: 85 - protein, 20 aa, Homo sapiens
LTCGEPPTCSPDQFACATGE

SEQ ID NO: 86 - protein, 20 aa, Homo sapiens
TCGEPPTCSPDQFACATGEI

SEQ ID NO: 87 - protein, 20 aa, Homo sapiens
CGEPPTCSPDQFACATGEID

SEQ ID NO: 88 - protein, 20 aa, Homo sapiens
GEPPTCSPDQFACATGEIDC

SEQ ID NO: 89 - protein, 20 aa, Homo sapiens
EPPTCSPDQFACATGEIDCI

SEQ ID NO: 90 - protein, 20 aa, Homo sapiens
PPTCSPDQFACATGEIDCIP

SEQ ID NO: 91 - protein, 20 aa, Homo sapiens
PTCSPDQFACATGEIDCIPG

SEQ ID NO: 92 - protein, 20 aa, Homo sapiens
TCSPDQFACATGEIDCIPGA

SEQ ID NO: 93 - protein, 20 aa, Homo sapiens
CSPDQFACATGEIDCIPGAW

SEQ ID NO: 94 - protein, 20 aa, Homo sapiens
SPDQFACATGEIDCIPGAWR

SEQ ID NO: 95 - protein, 20 aa, Homo sapiens
PDQFACATGEIDCIPGAWRC

SEQ ID NO: 96 - protein, 20 aa, Homo sapiens
DQFACATGEIDCIPGAWRCD

SEQ ID NO: 97 - protein, 20 aa, Homo sapiens
QFACATGEIDCIPGAWRCDG

SEQ ID NO: 98 - protein, 20 aa, Homo sapiens
FACATGEIDCIPGAWRCDGF

SEQ ID NO: 99 - protein, 20 aa, Homo sapiens
ACATGEIDCIPGAWRCDGFP

SEQ ID NO: 100 - protein, 20 aa, Homo sapiens
CATGEIDCIPGAWRCDGFPE

SEQ ID NO: 101 - protein, 20 aa, Homo sapiens
ATGEIDCIPGAWRCDGFPEC

SEQ ID NO: 102 - protein, 20 aa, Homo sapiens
TGEIDCIPGAWRCDGFPECD

SEQ ID NO: 103 - protein, 20 aa, Homo sapiens
GEIDCIPGAWRCDGFPECDD

SEQ ID NO: 104 - protein, 20 aa, Homo sapiens
EIDCIPGAWRCDGFPECDDQ

SEQ ID NO: 105 - protein, 20 aa, Homo sapiens
IDCIPGAWRCDGFPECDDQS

SEQ ID NO: 106 - protein, 20 aa, Homo sapiens
DCIPGAWRCDGFPECDDQSD

SEQ ID NO: 107 - protein, 20 aa, Homo sapiens
CIPGAWRCDGFPECDDQSDE

SEQ ID NO: 108 - protein, 20 aa, Homo sapiens
IPGAWRCDGFPECDDQSDEE

SEQ ID NO: 109 - protein, 20 aa, Homo sapiens
PGAWRCDGFPECDDQSDEEG

SEQ ID NO: 110 - protein, 20 aa, Homo sapiens
GAWRCDGFPECDDQSDEEGC

SEQ ID NO: 111 - protein, 20 aa, Homo sapiens
AWRCDGFPECDDQSDEEGCP

SEQ ID NO: 112 - protein, 20 aa, Homo sapiens
WRCDGFPECDDQSDEEGCPV

SEQ ID NO: 113 - protein, 20 aa, Homo sapiens
RCDGFPECDDQSDEEGCPVC

SEQ ID NO: 114 - protein, 20 aa, Homo sapiens
CDGFPECDDQSDEEGCPVCS

SEQ ID NO: 115 - protein, 20 aa, Homo sapiens
DGFPECDDQSDEEGCPVCSA

SEQ ID NO: 116 - protein, 20 aa, Homo sapiens
GFPECDDQSDEEGCPVCSAA

SEQ ID NO: 117 - protein, 20 aa, Homo sapiens
FPECDDQSDEEGCPVCSAAQ

SEQ ID NO: 118 - protein, 20 aa, Homo sapiens
PECDDQSDEEGCPVCSAAQF

SEQ ID NO: 119 - protein, 20 aa, Homo sapiens
ECDDQSDEEGCPVCSAAQFP

SEQ ID NO: 120 - protein, 20 aa, Homo sapiens
CDDQSDEEGCPVCSAAQFPC

SEQ ID NO: 121 - protein, 20 aa, Homo sapiens
DDQSDEEGCPVCSAAQFPCA

SEQ ID NO: 122 - protein, 20 aa, Homo sapiens
DQSDEEGCPVCSAAQFPCAR

SEQ ID NO: 123 - protein, 20 aa, Homo sapiens
QSDEEGCPVCSAAQFPCARG

SEQ ID NO: 124 - protein, 20 aa, Homo sapiens
SDEEGCPVCSAAQFPCARGQ

SEQ ID NO: 125 - protein, 20 aa, Homo sapiens
DEEGCPVCSAAQFPCARGQC

SEQ ID NO: 126 - protein, 20 aa, Homo sapiens
EEGCPVCSAAQFPCARGQCV

SEQ ID NO: 127 - protein, 20 aa, Homo sapiens
EGCPVCSAAQFPCARGQCVD

SEQ ID NO: 128 - protein, 20 aa, Homo sapiens
GCPVCSAAQFPCARGQCVDL

SEQ ID NO: 129 - protein, 20 aa, Homo sapiens
CPVCSAAQFPCARGQCVDLR

SEQ ID NO: 130 - protein, 20 aa, Homo sapiens
PVCSAAQFPCARGQCVDLRL

SEQ ID NO: 131 - protein, 20 aa, Homo sapiens
VCSAAQFPCARGQCVDLRLR

SEQ ID NO: 132 - protein, 20 aa, Homo sapiens
CSAAQFPCARGQCVDLRLRC

SEQ ID NO: 133 - protein, 20 aa, Homo sapiens
SAAQFPCARGQCVDLRLRCD

SEQ ID NO: 134 - protein, 20 aa, Homo sapiens
GCPVCSAAQFPCARGQCVDL

SEQ ID NO: 135 - protein, 20 aa, Homo sapiens
AQFPCARGQCVDLRLRCDGE

SEQ ID NO: 136 - protein, 20 aa, Homo sapiens
QFPCARGQCVDLRLRCDGEA

SEQ ID NO: 137 - protein, 20 aa, Homo sapiens
FPCARGQCVDLRLRCDGEAD

SEQ ID NO: 138 - protein, 20 aa, Homo sapiens
PCARGQCVDLRLRCDGEADC

SEQ ID NO: 139 - protein, 20 aa, Homo sapiens
CARGQCVDLRLRCDGEADCQ

SEQ ID NO: 140 - protein, 20 aa, Homo sapiens
ARGQCVDLRLRCDGEADCQD

SEQ ID NO: 141 - protein, 20 aa, Homo sapiens
RGQCVDLRLRCDGEADCQDR

SEQ ID NO: 142 - protein, 20 aa, Homo sapiens
GQCVDLRLRCDGEADCQDRS

SEQ ID NO: 143 - protein, 20 aa, Homo sapiens
QCVDLRLRCDGEADCQDRSD

SEQ ID NO: 144 - protein, 20 aa, Homo sapiens
CVDLRLRCDGEADCQDRSDE

SEQ ID NO: 145 - protein, 20 aa, Homo sapiens
VDLRLRCDGEADCQDRSDEA

SEQ ID NO: 146 - protein, 20 aa, Homo sapiens
DLRLRCDGEADCQDRSDEAD

SEQ ID NO: 147 - protein, 20 aa, Homo sapiens
LRLRCDGEADCQDRSDEADC

SEQ ID NO: 148 - protein, 20 aa, Homo sapiens
RLRCDGEADCQDRSDEADCD

SEQ ID NO: 149 - protein, 20 aa, Homo sapiens
LRCDGEADCQDRSDEADCDA

SEQ ID NO: 150 - protein, 20 aa, Homo sapiens
RCDGEADCQDRSDEADCDAI

SEQ ID NO: 151 - protein, 20 aa, Homo sapiens
CDGEADCQDRSDEADCDAIC

SEQ ID NO: 152 - protein, 20 aa, Homo sapiens
DGEADCQDRSDEADCDAICL

SEQ ID NO: 153 - protein, 20 aa, Homo sapiens
GEADCQDRSDEADCDAICLP

SEQ ID NO: 154 - protein, 20 aa, Homo sapiens
EADCQDRSDEADCDAICLPN

SEQ ID NO: 155 - protein, 20 aa, Homo sapiens
ADCQDRSDEADCDAICLPNQ

SEQ ID NO: 156 - protein, 20 aa, Homo sapiens
DCQDRSDEADCDAICLPNQF

SEQ ID NO: 157 - protein, 20 aa, Homo sapiens
CQDRSDEADCDAICLPNQFR

SEQ ID NO: 158 - protein, 20 aa, Homo sapiens
QDRSDEADCDAICLPNQFRC

SEQ ID NO: 159 - protein, 20 aa, Homo sapiens
DRSDEADCDAICLPNQFRCA

SEQ ID NO: 160 - protein, 20 aa, Homo sapiens
RSDEADCDAICLPNQFRCAS

SEQ ID NO: 161 - protein, 20 aa, Homo sapiens
SDEADCDAICLPNQFRCASG

SEQ ID NO: 162 - protein, 20 aa, Homo sapiens
DEADCDAICLPNQFRCASGQ

SEQ ID NO: 163 - protein, 20 aa, Homo sapiens
EADCDAICLPNQFRCASGQC

SEQ ID NO: 164 - protein, 20 aa, Homo sapiens
ADCDAICLPNQFRCASGQCV

SEQ ID NO: 165 - protein, 20 aa, Homo sapiens
DCDAICLPNQFRCASGQCVL

SEQ ID NO: 166 - protein, 20 aa, Homo sapiens
CDAICLPNQFRCASGQCVLI

SEQ ID NO: 167 - protein, 20 aa, Homo sapiens
DAICLPNQFRCASGQCVLIK

SEQ ID NO: 168 - protein, 20 aa, Homo sapiens
AICLPNQFRCASGQCVLIKQ

SEQ ID NO: 169 - protein, 20 aa, Homo sapiens
ICLPNQFRCASGQCVLIKQQ

SEQ ID NO: 170 - protein, 20 aa, Homo sapiens
CLPNQFRCASGQCVLIKQQC

SEQ ID NO: 171 - protein, 20 aa, Homo sapiens
LPNQFRCASGQCVLIKQQCD

SEQ ID NO: 172 - protein, 20 aa, Homo sapiens
PNQFRCASGQCVLIKQQCDS

SEQ ID NO: 173 - protein, 20 aa, Homo sapiens
NQFRCASGQCVLIKQQCDSF

SEQ ID NO: 174 - protein, 20 aa, Homo sapiens
QFRCASGQCVLIKQQCDSFP

SEQ ID NO: 175 - protein, 20 aa, Homo sapiens
FRCASGQCVLIKQQCDSFPD

SEQ ID NO: 176 - protein, 20 aa, Homo sapiens
RCASGQCVLIKQQCDSFPDC

SEQ ID NO: 177 - protein, 20 aa, Homo sapiens
CASGQCVLIKQQCDSFPDCI

SEQ ID NO: 178 - protein, 20 aa, Homo sapiens
ASGQCVLIKQQCDSFPDCID

SEQ ID NO: 179 - protein, 20 aa, Homo sapiens
SGQCVLIKQQCDSFPDCIDG

SEQ ID NO: 180 - protein, 20 aa, Homo sapiens
GQCVLIKQQCDSFPDCIDGS

SEQ ID NO: 181 - protein, 20 aa, Homo sapiens
QCVLIKQQCDSFPDCIDGSD

SEQ ID NO: 182 - protein, 20 aa, Homo sapiens
CVLIKQQCDSFPDCIDGSDE

SEQ ID NO: 183 - protein, 20 aa, Homo sapiens
VLIKQQCDSFPDCIDGSDEL

SEQ ID NO: 184 - protein, 20 aa, Homo sapiens
KGDGTTRCSCPMHLVLLQDE

SEQ ID NO: 185 - protein, 20 aa, Homo sapiens
GDGTTRCSCPMHLVLLQDEL

SEQ ID NO: 186 - protein, 20 aa, Homo sapiens
DGTTRCSCPMHLVLLQDELS

SEQ ID NO: 187 - protein, 20 aa, Homo sapiens
GTTRCSCPMHLVLLQDELSC

SEQ ID NO: 188 - protein, 20 aa, Homo sapiens
TTRCSCPMHLVLLQDELSCG

SEQ ID NO: 189 - protein, 20 aa, Homo sapiens
TRCSCPMHLVLLQDELSCGE

SEQ ID NO: 190 - protein, 20 aa, Homo sapiens
RCSCPMHLVLLQDELSCGEP

SEQ ID NO: 191 - protein, 20 aa, Homo sapiens
CSCPMHLVLLQDELSCGEPP

SEQ ID NO: 192 - protein, 20 aa, Homo sapiens
SCPMHLVLLQDELSCGEPPT

SEQ ID NO: 193 - protein, 20 aa, Homo sapiens
CPMHLVLLQDELSCGEPPTC

SEQ ID NO: 194 - protein, 20 aa, Homo sapiens
PMHLVLLQDELSCGEPPTCS

SEQ ID NO: 195 - protein, 20 aa, Homo sapiens
MHLVLLQDELSCGEPPTCSP

SEQ ID NO: 196 - protein, 20 aa, Homo sapiens
HLVLLQDELSCGEPPTCSPQ

SEQ ID NO: 197 - protein, 20 aa, Homo sapiens
LVLLQDELSCGEPPTCSPQQ

SEQ ID NO: 198 - protein, 20 aa, Homo sapiens
VLLQDELSCGEPPTCSPQQF

SEQ ID NO: 199 - protein, 20 aa, Homo sapiens
LLQDELSCGEPPTCSPQQFT

SEQ ID NO: 200 - protein, 20 aa, Homo sapiens
LQDELSCGEPPTCSPQQFTC

SEQ ID NO: 201 - protein, 20 aa, Homo sapiens
QDELSCGEPPTCSPQQFTCF

SEQ ID NO: 202 - protein, 20 aa, Homo sapiens
DELSCGEPPTCSPQQFTCFT

SEQ ID NO: 203 - protein, 20 aa, Homo sapiens
ELSCGEPPTCSPQQFTCFTG

SEQ ID NO: 204 - protein, 20 aa, Homo sapiens
LSCGEPPTCSPQQFTCFTGE

SEQ ID NO: 205 - protein, 20 aa, Homo sapiens
SCGEPPTCSPQQFTCFTGEI

SEQ ID NO: 206 - protein, 20 aa, Homo sapiens
CGEPPTCSPQQFTCFTGEID

SEQ ID NO: 207 - protein, 20 aa, Homo sapiens
GEPPTCSPQQFTCFTGEIDC

SEQ ID NO: 208 - protein, 20 aa, Homo sapiens
EPPTCSPQQFTCFTGEIDCI

SEQ ID NO: 209 - protein, 20 aa, Homo sapiens
PPTCSPQQFTCFTGEIDCIP

SEQ ID NO: 210 - protein, 20 aa, Homo sapiens
PTCSPQQFTCFTGEIDCIPV

SEQ ID NO: 211 - protein, 20 aa, Homo sapiens
TCSPQQFTCFTGEIDCIPVA

SEQ ID NO: 212 - protein, 20 aa, Homo sapiens
CSPQQFTCFTGEIDCIPVAW

SEQ ID NO: 213 - protein, 20 aa, Homo sapiens
SPQQFTCFTGEIDCIPVAWR

SEQ ID NO: 214 - protein, 20 aa, Homo sapiens
PQQFTCFTGEIDCIPVAWRC

SEQ ID NO: 215 - protein, 20 aa, Homo sapiens
QQFTCFTGEIDCIPVAWRCD

SEQ ID NO: 216 - protein, 20 aa, Homo sapiens
QFTCFTGEIDCIPVAWRCDG

SEQ ID NO: 217 - protein, 20 aa, Homo sapiens
FTCFTGEIDCIPVAWRCDGF

SEQ ID NO: 218 - protein, 20 aa, Homo sapiens
TCFTGEIDCIPVAWRCDGFT

SEQ ID NO: 219 - protein, 20 aa, Homo sapiens
CFTGEIDCIPVAWRCDGFTE

SEQ ID NO: 220 - protein, 20 aa, Homo sapiens
FTGEIDCIPVAWRCDGFTEC

SEQ ID NO: 221 - protein, 20 aa, Homo sapiens
TGEIDCIPVAWRCDGFTECE

SEQ ID NO: 222 - protein, 20 aa, Homo sapiens
GEIDCIPVAWRCDGFTECED

SEQ ID NO: 223 - protein, 20 aa, Homo sapiens
EIDCIPVAWRCDGFTECEDH

SEQ ID NO: 224 - protein, 20 aa, Homo sapiens
IDCIPVAWRCDGFTECEDHS

SEQ ID NO: 225 - protein, 20 aa, Homo sapiens
DCIPVAWRCDGFTECEDHSD

SEQ ID NO: 226 - protein, 20 aa, Homo sapiens
CIPVAWRCDGFTECEDHSDE

SEQ ID NO: 227 - protein, 20 aa, Homo sapiens
IPVAWRCDGFTECEDHSDEL

SEQ ID NO: 228 - protein, 20 aa, Homo sapiens
PVAWRCDGFTECEDHSDELN

SEQ ID NO: 229 - protein, 20 aa, Homo sapiens
VAWRCDGFTECEDHSDELNC

SEQ ID NO: 230 - protein, 20 aa, Homo sapiens
AWRCDGFTECEDHSDELNCP

SEQ ID NO: 231 - protein, 20 aa, Homo sapiens
WRCDGFTECEDHSDELNCPV

SEQ ID NO: 232 - protein, 20 aa, Homo sapiens
RCDGFTECEDHSDELNCPVC

SEQ ID NO: 233 - protein, 20 aa, Homo sapiens
CDGFTECEDHSDELNCPVCS

SEQ ID NO: 234 - protein, 20 aa, Homo sapiens
DGFTECEDHSDELNCPVCSE

SEQ ID NO: 235 - protein, 20 aa, Homo sapiens
GFTECEDHSDELNCPVCSES

SEQ ID NO: 236 - protein, 20 aa, Homo sapiens
FTECEDHSDELNCPVCSESQ

SEQ ID NO: 237 - protein, 20 aa, Homo sapiens
TECEDHSDELNCPVCSESQF

SEQ ID NO: 238 - protein, 20 aa, Homo sapiens
ECEDHSDELNCPVCSESQFQ

SEQ ID NO: 239 - protein, 20 aa, Homo sapiens
CEDHSDELNCPVCSESQFQC

SEQ ID NO: 240 - protein, 20 aa, Homo sapiens
EDHSDELNCPVCSESQFQCA

SEQ ID NO: 241 - protein, 20 aa, Homo sapiens
DHSDELNCPVCSESQFQCAS

SEQ ID NO: 242 - protein, 20 aa, Homo sapiens
HSDELNCPVCSESQFQCASG

SEQ ID NO: 243 - protein, 20 aa, Homo sapiens
SDELNCPVCSESQFQCASGQ

SEQ ID NO: 244 - protein, 20 aa, Homo sapiens
DELNCPVCSESQFQCASGQC

SEQ ID NO: 245 - protein, 20 aa, Homo sapiens
ELNCPVCSESQFQCASGQCI

SEQ ID NO: 246 - protein, 20 aa, Homo sapiens
LNCPVCSESQFQCASGQCID

SEQ ID NO: 247 - protein, 20 aa, Homo sapiens
NCPVCSESQFQCASGQCIDG

SEQ ID NO: 248 - protein, 20 aa, Homo sapiens
CPVCSESQFQCASGQCIDGA

SEQ ID NO: 249 - protein, 20 aa, Homo sapiens
PVCSESQFQCASGQCIDGAL

SEQ ID NO: 250 - protein, 20 aa, Homo sapiens
VCSESQFQCASGQCIDGALR

SEQ ID NO: 251 - protein, 20 aa, Homo sapiens
CSESQFQCASGQCIDGALRC

SEQ ID NO: 252 - protein, 20 aa, Homo sapiens
SESQFQCASGQCIDGALRCN

SEQ ID NO: 253 - protein, 20 aa, Homo sapiens
ESQFQCASGQCIDGALRCNG

SEQ ID NO: 254 - protein, 20 aa, Homo sapiens
SQFQCASGQCIDGALRCNGD

SEQ ID NO: 255 - protein, 20 aa, Homo sapiens
QFQCASGQCIDGALRCNGDA

SEQ ID NO: 256 - protein, 20 aa, Homo sapiens
FQCASGQCIDGALRCNGDAN

SEQ ID NO: 257 - protein, 20 aa, Homo sapiens
QCASGQCIDGALRCNGDANC

SEQ ID NO: 258 - protein, 20 aa, Homo sapiens
CASGQCIDGALRCNGDANCQ

SEQ ID NO: 259 - protein, 20 aa, Homo sapiens
ASGQCIDGALRCNGDANCQD

SEQ ID NO: 260 - protein, 20 aa, Homo sapiens
SGQCIDGALRCNGDANCQDK

SEQ ID NO: 261 - protein, 20 aa, Homo sapiens
GQCIDGALRCNGDANCQDKS

SEQ ID NO: 262 - protein, 20 aa, Homo sapiens
QCIDGALRCNGDANCQDKSD

SEQ ID NO: 263 - protein, 20 aa, Homo sapiens
CIDGALRCNGDANCQDKSDE

SEQ ID NO: 264 - protein, 20 aa, Homo sapiens
IDGALRCNGDANCQDKSDEK

SEQ ID NO: 265 - protein, 20 aa, Homo sapiens
DGALRCNGDANCQDKSDEKN

SEQ ID NO: 266 - protein, 20 aa, Homo sapiens
GALRCNGDANCQDKSDEKNC

SEQ ID NO: 267 - protein, 20 aa, Homo sapiens
ALRCNGDANCQDKSDEKNCE

SEQ ID NO: 268 - protein, 20 aa, Homo sapiens
LRCNGDANCQDKSDEKNCEV

SEQ ID NO: 269 - protein, 20 aa, Homo sapiens
RCNGDANCQDKSDEKNCEVL

SEQ ID NO: 270 - protein, 20 aa, Homo sapiens
CNGDANCQDKSDEKNCEVLC

SEQ ID NO: 271 - protein, 20 aa, Homo sapiens
NGDANCQDKSDEKNCEVLCL

SEQ ID NO: 272 - protein, 20 aa, Homo sapiens
GDANCQDKSDEKNCEVLCLI

SEQ ID NO: 273 - protein, 20 aa, Homo sapiens
DANCQDKSDEKNCEVLCLID

SEQ ID NO: 274 - protein, 20 aa, Homo sapiens
ANCQDKSDEKNCEVLCLIDQ

SEQ ID NO: 275 - protein, 20 aa, Homo sapiens
NCQDKSDEKNCEVLCLIDQF

SEQ ID NO: 276 - protein, 20 aa, Homo sapiens
CQDKSDEKNCEVLCLIDQFR

SEQ ID NO: 277 - protein, 20 aa, Homo sapiens
QDKSDEKNCEVLCLIDQFRC

SEQ ID NO: 278 - protein, 20 aa, Homo sapiens
DKSDEKNCEVLCLIDQFRCA

SEQ ID NO: 279 - protein, 20 aa, Homo sapiens
KSDEKNCEVLCLIDQFRCAN

SEQ ID NO: 280 - protein, 20 aa, Homo sapiens
SDEKNCEVLCLIDQFRCANG

SEQ ID NO: 281 - protein, 20 aa, Homo sapiens
DEKNCEVLCLIDQFRCANGQ

SEQ ID NO: 282 - protein, 20 aa, Homo sapiens
EKNCEVLCLIDQFRCANGQC

SEQ ID NO: 283 - protein, 20 aa, Homo sapiens
KNCEVLCLIDQFRCANGQCI

SEQ ID NO: 284 - protein, 20 aa, Homo sapiens
NCEVLCLIDQFRCANGQCIG

SEQ ID NO: 285 - protein, 20 aa, Homo sapiens
CEVLCLIDQFRCANGQCIGK

SEQ ID NO: 286 - protein, 20 aa, Homo sapiens
EVLCLIDQFRCANGQCIGKH

SEQ ID NO: 287 - protein, 20 aa, Homo sapiens
VLCLIDQFRCANGQCIGKHK

SEQ ID NO: 288 - protein, 20 aa, Homo sapiens
LCLIDQFRCANGQCIGKHKK

SEQ ID NO: 289 - protein, 20 aa, Homo sapiens
CLIDQFRCANGQCIGKHKKC

SEQ ID NO: 290 - protein, 20 aa, Homo sapiens
LIDQFRCANGQCIGKHKKCD

SEQ ID NO: 291 - protein, 20 aa, Homo sapiens
IDQFRCANGQCIGKHKKCDH

SEQ ID NO: 292 - protein, 20 aa, Homo sapiens
DQFRCANGQCIGKHKKCDHN

SEQ ID NO: 293 - protein, 20 aa, Homo sapiens
QFRCANGQCIGKHKKCDHNV

SEQ ID NO: 294 - protein, 20 aa, Homo sapiens
FRCANGQCIGKHKKCDHNVD

SEQ ID NO: 295 - protein, 20 aa, Homo sapiens
RCANGQCIGKHKKCDHNVDC

SEQ ID NO: 296 - protein, 20 aa, Homo sapiens
CANGQCIGKHKKCDHNVDCS

SEQ ID NO: 297 - protein, 20 aa, Homo sapiens
ANGQCIGKHKKCDHNVDCSD

SEQ ID NO: 298 - protein, 20 aa, Homo sapiens
NGQCIGKHKKCDHNVDCSDD

SEQ ID NO: 299 - protein, 20 aa, Homo sapiens
GQCIGKHKKCDHNVDCSDDE

SEQ ID NO: 300 - protein, 20 aa, Homo sapiens
QCIGKHKKCDHNVDCSDDEL

SEQ ID NO: 301 - protein, 20 aa, Homo sapiens
RISLETNNNDVAIPLTGVKE

SEQ ID NO: 302 - protein, 20 aa, Homo sapiens
ISLETNNNDVAIPLTGVKEA

SEQ ID NO: 303 - protein, 20 aa, Homo sapiens
SLETNNNDVAIPLTGVKEAS

SEQ ID NO: 304 - protein, 20 aa, Homo sapiens
LETNNNDVAIPLTGVKEASA

SEQ ID NO: 305 - protein, 20 aa, Homo sapiens
ETNNNDVAIPLTGVKEASAL

SEQ ID NO: 306 - protein, 20 aa, Homo sapiens
TNNNDVAIPLTGVKEASALD

SEQ ID NO: 307 - protein, 20 aa, Homo sapiens
NNNDVAIPLTGVKEASALDF

SEQ ID NO: 308 - protein, 20 aa, Homo sapiens
NNDVAIPLTGVKEASALDFD

SEQ ID NO: 309 - protein, 20 aa, Homo sapiens
NDVAIPLTGVKEASALDFDV

SEQ ID NO: 310 - protein, 20 aa, Homo sapiens
DVAIPLTGVKEASALDFDVS

SEQ ID NO: 311 - protein, 20 aa, Homo sapiens
VAIPLTGVKEASALDFDVSN

SEQ ID NO: 312 - protein, 20 aa, Homo sapiens
AIPLTGVKEASALDFDVSNN

SEQ ID NO: 313 - protein, 20 aa, Homo sapiens
IPLTGVKEASALDFDVSNNH

SEQ ID NO: 314 - protein, 20 aa, Homo sapiens
PLTGVKEASALDFDVSNNHI

SEQ ID NO: 315 - protein, 20 aa, Homo sapiens
LTGVKEASALDFDVSNNHIY

SEQ ID NO: 316 - protein, 20 aa, Homo sapiens
TGVKEASALDFDVSNNHIYW

SEQ ID NO: 317 - protein, 20 aa, Homo sapiens
GVKEASALDFDVSNNHIYWT

SEQ ID NO: 318 - protein, 20 aa, Homo sapiens
VKEASALDFDVSNNHIYWTD

SEQ ID NO: 319 - protein, 20 aa, Homo sapiens
KEASALDFDVSNNHIYWTDV

SEQ ID NO: 320 - protein, 20 aa, Homo sapiens
EASALDFDVSNNHIYWTDVS

SEQ ID NO: 321 - protein, 20 aa, Homo sapiens
ASALDFDVSNNHIYWTDVSL

SEQ ID NO: 322 - protein, 20 aa, Homo sapiens
SALDFDVSNNHIYWTDVSLK

SEQ ID NO: 323 - protein, 20 aa, Homo sapiens
ALDFDVSNNHIYWTDVSLKT

SEQ ID NO: 324 - protein, 20 aa, Homo sapiens
RISLETNNNNVAIPLTGVKE

SEQ ID NO: 325 - protein, 20 aa, Homo sapiens
ISLETNNNNVAIPLTGVKEA

SEQ ID NO: 326 - protein, 20 aa, Homo sapiens
SLETNNNNVAIPLTGVKEAS

SEQ ID NO: 327 - protein, 20 aa, Homo sapiens
LETNNNNVAIPLTGVKEASA

SEQ ID NO: 328 - protein, 20 aa, Homo sapiens
ETNNNNVAIPLTGVKEASAL

SEQ ID NO: 329 - protein, 20 aa, Homo sapiens
TNNNNVAIPLTGVKEASALD

SEQ ID NO: 330 - protein, 20 aa, Homo sapiens
NNNNVAIPLTGVKEASALDF

SEQ ID NO: 331 - protein, 20 aa, Homo sapiens
NNNVAIPLTGVKEASALDFD

SEQ ID NO: 332 - protein, 20 aa, Homo sapiens
NNVAIPLTGVKEASALDFDV

SEQ ID NO: 333 - protein, 20 aa, Homo sapiens
NVAIPLTGVKEASALDFDVT

SEQ ID NO: 334 - protein, 20 aa, Homo sapiens
VAIPLTGVKEASALDFDVTN

SEQ ID NO: 335 - protein, 20 aa, Homo sapiens
AIPLTGVKEASALDFDVTND

SEQ ID NO: 336 - protein, 20 aa, Homo sapiens
IPLTGVKEASALDFDVTNDR

SEQ ID NO: 337 - protein, 20 aa, Homo sapiens
PLTGVKEASALDFDVTNDRI

SEQ ID NO: 338 - protein, 20 aa, Homo sapiens
LTGVKEASALDFDVTNDRIY

SEQ ID NO: 339 - protein, 20 aa, Homo sapiens
TGVKEASALDFDVTNDRIYW

SEQ ID NO: 340 - protein, 20 aa, Homo sapiens
GVKEASALDFDVTNDRIYWT

SEQ ID NO: 341 - protein, 20 aa, Homo sapiens
VKEASALDFDVTNDRIYWTD

SEQ ID NO: 342 - protein, 20 aa, Homo sapiens
KEASALDFDVTNDRIYWTDI

SEQ ID NO: 343 - protein, 20 aa, Homo sapiens
EASALDFDVTNDRIYWTDIS

SEQ ID NO: 344 - protein, 20 aa, Homo sapiens
ASALDFDVTNDRIYWTDISL

SEQ ID NO: 345 - protein, 20 aa, Homo sapiens
SALDFDVTNDRIYWTDISLK

SEQ ID NO: 346 - protein, 20 aa, Homo sapiens
ALDFDVTNDRIYWTDISLKT

SEQ ID NO: 347 - protein, 20 aa, Homo sapiens
ERVHKVKASRDVIIDQLPDL

SEQ ID NO: 348 - protein, 20 aa, Homo sapiens
RVHKVKASRDVIIDQLPDLM

SEQ ID NO: 349 - protein, 20 aa, Homo sapiens
VHKVKASRDVIIDQLPDLMG

SEQ ID NO: 350 - protein, 20 aa, Homo sapiens
HKVKASRDVIIDQLPDLMGL

SEQ ID NO: 351 - protein, 20 aa, Homo sapiens
KVKASRDVIIDQLPDLMGLK

SEQ ID NO: 352 - protein, 20 aa, Homo sapiens
VKASRDVIIDQLPDLMGLKA

SEQ ID NO: 353 - protein, 20 aa, Homo sapiens
KASRDVIIDQLPDLMGLKAV

SEQ ID NO: 354 - protein, 20 aa, Homo sapiens
ASRDVIIDQLPDLMGLKAVN

SEQ ID NO: 355 - protein, 20 aa, Homo sapiens
SRDVIIDQLPDLMGLKAVNV

SEQ ID NO: 356 - protein, 20 aa, Homo sapiens
RDVIIDQLPDLMGLKAVNVA

SEQ ID NO: 357 - protein, 20 aa, Homo sapiens
DVIIDQLPDLMGLKAVNVAK

SEQ ID NO: 358 - protein, 20 aa, Homo sapiens
VIIDQLPDLMGLKAVNVAKV

SEQ ID NO: 359 - protein, 20 aa, Homo sapiens
IIDQLPDLMGLKAVNVAKVV

SEQ ID NO: 360 - protein, 20 aa, Homo sapiens
IDQLPDLMGLKAVNVAKVVG

SEQ ID NO: 361 - protein, 20 aa, Homo sapiens
DQLPDLMGLKAVNVAKVVGT

SEQ ID NO: 362 - protein, 20 aa, Homo sapiens
QLPDLMGLKAVNVAKVVGTN

SEQ ID NO: 363 - protein, 20 aa, Homo sapiens
ERVHKRSAEREVIIDQLPDL

SEQ ID NO: 364 - protein, 20 aa, Homo sapiens
RVHKRSAEREVIIDQLPDLM

SEQ ID NO: 365 - protein, 20 aa, Homo sapiens
VHKRSAEREVIIDQLPDLMG

SEQ ID NO: 366 - protein, 20 aa, Homo sapiens
HKRSAEREVIIDQLPDLMGL

SEQ ID NO: 367 - protein, 20 aa, Homo sapiens
KRSAEREVIIDQLPDLMGLK

SEQ ID NO: 368 - protein, 20 aa, Homo sapiens
RSAEREVIIDQLPDLMGLKA

SEQ ID NO: 369 - protein, 20 aa, Homo sapiens
SAEREVIIDQLPDLMGLKAT

SEQ ID NO: 370 - protein, 20 aa, Homo sapiens
AEREVIIDQLPDLMGLKATN

SEQ ID NO: 371 - protein, 20 aa, Homo sapiens
EREVIIDQLPDLMGLKATNV

SEQ ID NO: 372 - protein, 20 aa, Homo sapiens
REVIIDQLPDLMGLKATNVH

SEQ ID NO: 373 - protein, 20 aa, Homo sapiens
EVIIDQLPDLMGLKATNVHR

SEQ ID NO: 374 - protein, 20 aa, Homo sapiens
VIIDQLPDLMGLKATNVHRV

SEQ ID NO: 375 - protein, 20 aa, Homo sapiens
IIDQLPDLMGLKATNVHRVI

SEQ ID NO: 376 - protein, 20 aa, Homo sapiens
IDQLPDLMGLKATNVHRVIG

SEQ ID NO: 377 - protein, 20 aa, Homo sapiens
DQLPDLMGLKATNVHRVIGS

SEQ ID NO: 378 - protein, 20 aa, Homo sapiens
QLPDLMGLKATNVHRVIGSN

SEQ ID NO: 379 - DNA, 7500 bp, Mus musculus
ggaaccctccgctgattctctccgccccggcgtcccttgggagcccgcgggacccccgggccggcagggaacgccggcggcggccgcagcaccatgaggcggtggtggggcgcgctgctgcttggcgccctgctctgcgcacacggaatagccagcagtctggagtgtgcatgtggtcggagccacttcacatgtgcggtgagcgcgcttggcgaatgtacctgtatccccgcccagtggcagtgtgatggagacaatgactgtggagaccacagcgatgaggatggctgtacgctgcctacctgctcccctctggacttccactgtgacaacggcaagtgcatccgacgctcctgggtgtgtgacggggacaatgactgtgaagatgactctgatgagcaggactgtcccccccgggagtgtgaggaggacgaattcccctgccagaacggctactgcattcggagtctgtggcactgtgatggggacaacgactgtggtgacaacagtgacgagcaatgcgacatgcgcaagtgctcagacaaggaattccgctgcagcgacggcagctgcattgctgagcattggtactgcgatggtgacacagactgcaaagatggctctgatgaggaaagctgcccttcagcagtaccctctcctccttgcaacctggaagagttccagtgcgcctatggccgctgcatcctcgacatctaccactgtgacggagatgacgactgtggagactggtcagacgagtctgactgctcctcccaccagccctgccgctctggggaattcatgtgtgacagtggcctgtgcatcaactcgggctggcgctgtgatggtgatgcagactgtgatgaccagtctgatgaacgcaactgcaccacctccatgtgcacagccgaacagtttcgctgtcggtcaggccgttgcgtccgtctgtcctggcgctgtgatggggaggatgactgtgcggacaacagcgatgaagaaaactgtgagaacacaggaagcccccagtgcgcctctgaccagttcctgtgttggaacgggcgctgtattgggcagaggaagctgtgcaacgggatcaacgactgcggtgacagcagtgacgaaagtccacagcagaactgtcggccccggacgggtgaggagaactgcaatgttaacaacggtggctgtgcccagaagtgtcagatggtgcgaggggcagtgcagtgtacctgccacacaggctaccggctcacagaggatgggagaacgtgccaggatgtaaacgaatgtgctgaggaggggtactgcagccagggctgcacaaacactgaaggggctttccagtgctggtgtgaagcagggtatgagctacggcccgatcggcgcagctgcaaagctctggggccagaacctgtgctactgtttgccaatcgtatcgacatccggcaggtgctcccgcaccgctccgagtacacgctgctactgaacaacctggagaacgccattgccctcgactttcatcaccggcgggagctggtcttctggtcggatgtcaccctggaccgcatcctccgtgccaaccttaatggcagcaatgtagaggaggtggtgtctactgggctagagagcccagggggcctagcggtggattgggtccatgacaagctctactggaccgactctgggacgtcgaggattgaggtggctaatctggatggggcccaccggaaggtgctgctatggcagagcctagagaagcccagagccattgccctacaccccatggagggaaccatttattggacagactggggcaacaccccccgcattgaggcctccagcatggacggctctgggcgtcgcatcattgctgacactcatcttttctggcccaacggcctcaccatcgactacgctggtcgccgcatgtactgggtggatgctaagcaccacgtcatcgagagggccaatctagatgggagtcaccgcaaggctgtcattagccagggcctcccacatccctttgccatcacagtgtttgaagacagcttgtattggacagactggcacaccaagagcatcaatagtgccaacaagtttacggggaagaaccaggaaatcattcgcaacaagctccactttcctatggacattcataccttgcacccccagcgccagcccgcagggaaaaaccgctgtggggacaacaatggaggttgcactcacctgtgtctacccagtggccagaactacacctgtgcctgccccaccggcttccgcaagatcaacagccacgcctgtgcccagagtcttgacaagttcctgctttttgcccgaaggatggacatccgtcggatcagcttcgacacagaggacctgtccgacgatgtcatcccactggctgacgtgcgcagtgctgtggcccttgactgggactcccgggatgaccacgtgtattggacagatgtcagcactgataccatcagcagggccaagtgggatggaacaggacaggaggtggtagtggataccagtttggagagccctgctggcctggcaattgattgggtcaccaacaagctctactggacagatgcaggcacggacaggatcgaagtggccaacacagatggcagcatgaggacagtgctcatctgggagaaccttgaccgtcctcgggacatagtggtagagcccatgggcgggtacatgtattggactgactggggtgcaagtcccaagattgaacgggctggcatggacgcctccagccgacaagtcatcatctcttcgaacctgacctggcctaacggattggctattgactacggctctcagcggctgtactgggcggatgccggcatgaagactattgaatttgctgggctggatggcagcaagaggaaggtactaattggaagccagctcccccacccttttgggctgactctatatggacagcgcatctattggactgactggcagaccaagagtatacaaagtgctgaccggctgactgggctagaccgggagactctgcaggagaacctggagaacctcatggacattcatgtcttccaccgccagaggcctccagtgacgacgctgtgtgccgtggagaatggcggctgcagccatttgtgtctcaggtccccaaatccgagcggcttcagctgcacctgccccacaggtatcaacctgctacgagatggcaagacatgctcaccaggcatgaacagtttcctcatcttcgccaggaggattgatgttcgcatggtctccttggatatcccttattttgctgatgtggtggtacccatcaacatgaccatgaagaacaccatcgccattggagtggatccgctggaaggaaaagtgtactggtctgacagcacgctgcacaggatcagccgtgccagcctggatggctcacagcatgaggacatcatcactacaggtctgcagaccacagacgggcttgcagtggatgccatcggccggaaagtgtactggacagacacaggaacgaatcggattgaagttggcaacctggatgggtctatgcggaaagtgttggtgtggcagaaccttgacagtccccgggccattgtattataccatgaaatggggttcatgtactggacagactggggggagaacgccaaattagagcgttccggaatggatggctcagaccgtactgtgctcatcaacaacaacctagggtggcccaatggactgactgtggacaagaccagttcccaactgctgtgggctgatgcccatactgagagaattgaggtggcagacctgaatggcgctaatcggcatacactggtgtcaccagtccagcacccatatggcctcactctgcttgactcctatatctactggactgactggcagacccgcagcatccaccgggccgacaagagtactggcagcaatgtcatccttgtgaggtccaacctgccaggcctcatggacatccaagctgttgaccgggcacagccactgggttttaataaatgtggctcaagaaatggcggctgttcccacctctgtttacctcggccttctggtttctcctgtgcctgtcccactggcattcagctgaagggagacaggaagacctgtgacccctctccagaaacctacctgctcttctctagccgaggctctattcgccgtatctcactggacaccgatgaccacacagacgtgcacgttcctgttcctgggctcaacaatgtcatctccctagactatgacagtgtgcatggaaaggtctattacacagacgtgttcctggatgttatcaggcgagcagacctgaatggcagcaacatggagactgttattggacacgggcttaagaccactgacgggctggcagtggactgggttgccagaaacctgtactggacagataccggccgaaataccatcgaagcatctcggctagatggatcctgtcgcaaagtgctaatcaacaacagtctagacgagccccgggccattgctgttttccccagaaaggggtacctcttctggacagactggggccacattgccaagattgaacgggcaaacctggatggctctgagaggaaggtcctcatcaacacagacctaggctggcctaacggcctcaccttggactatgatacccgcaggatctactgggtagatgcacatctggatcggatcgagagtgctgacctcaatgggaagctgaggcaggtcttggtcagccatgtgtcccacccctttgccctcacacagcaggacaggtggatctactggacggactggcagaccaaatcgatccaacgtgtcgacaagtactcgggccgcaacaaggagacagtactggccaacgtggagggacttatggatattattgtggtgtcccctcagcggcagacagggaccaatgcctgcggtgtgaataatggtggctgtactcacctctgctttgcccgagcctctgactttgtgtgcgcgtgtcctgatgagcctgacggtcatccctgctcccttgtacctggcctcgtacctcctgctcccagagctaccagcatgaatgagaagagcccagtgctacccaacacgctgcccaccaccttacattcttctaccaccaagacccgcacctctctggagggggcaggaggaagatgctctgaaagggatgcccagctgggcctctgtgcacattccaatgaagccgtccctgctgctccaggtgaaggactgcacgtcagctatgccatcggtggactcctcagtatcctgctgattttgttggtgatcgcggctttgatgctatacagacacagaaaatccaagttcacggatcctggaatgggaaacctgacctacagcaacccctcctaccgaacttccactcaggaagtgaaacttgaagcagctcccaagccagccgtgtataatcagctgtgctataagaaagagggtggacctgaccacagctacaccaaggagaagatcaagattgtagagggaatccgcctcctggctggggatgacgctgaatggggtgacctcaagcaactgcgcagctccagggggggccttctacgggatcacgtatgcatgaagacagacacagtgtccatccaggccagctctggctccctagatgacacggagacagagcagctgcttcaggaagagcagtctgagtgtagcagtgttcacactgcagccactccggagagacggggctctctgccagacacaggctggaaacacgaacgcaagctctcctcagaaagccaggtctaagtgctgcccacattctcttccctgcctgcctgttcattctcctctgtggacttctgatccttgtgctcacataacaacgcgggccctttcccatgcacgcatcttcctcctcccaccccaaagtgctcccgagcccccatagaacctgttactcatctgagtccaaaactacctggggacaagaagtgagggcacatctcaagaatttagacctggattttaccatgaacttcacctcttgcactctatcctgggccccctgaagctgggctcagtgtgattccttgtcagcacagagctccgtctccctttgccctggcatcctatgtgcctgccccacagcaccgatgcaattcataagactctggtcttccccatcacagctggcatgatggcctccctgtgaactggcctaccaggggctgggactggaggtcacaggttaaaaggtaatggacagaggttgagagaaggaagaagcagcccagctggtctgggtgcttgggaaacctccagcctcaagtgttggtaattatgaaaaaccttggggggcggggattcaagccctggaatcatgtccattgctggcagtggacattctcctcatccagaagtcactgctttgtcagttgtgatacaggtgaagtgtgggaatggcttaggctcttcttcctcctgctggaatgtctggggccatttctaccttggataactggatagctcctgccatggcaactcactaataggaattccctcctggtgctgtgatgggccgtacttttcttaagcctttagctatgctgtgagcttgagtgaacattgggtgggccttggagagcagacttgtagaacgtttaagaagctctgcaaagaaagcatagcatcctacccgagcttcacctattctactcttctctcttccattctgtgggattctaagggaaacaattctgtcacagcctttcaaagactggctcaacattttcctaaatggtcagaaagagcaactagagagacacaagacacccaggggctcaggaggagaattgtgaggttgaacctcttgagttcattctcaagtgctcagggatccaagttcatggacacagcctgtggctctcgggtggtaaaggtcatgtgccaactcttctcagcccctgtccacactaaagtgccagcattagcttcatgctcctttaggccaagggcttctctcaggggagctgagattttttttttttttttgtactgccctagaatctcatcccagtttttctacttctgagagaggggagtctagccttgctttctctaaccggtcctgtgtcacctcaatcgctaatgtccaatgatgaggagggtcagtgaagatgtaaagtggtaagcaaggagtggagaggtggtgagaaccagtccagagcaagttcaggcacttctccatagaaaccgaagcacaggagagattggcaggtgctgagaatggaggagaggacttttaggggaagtttcctgacataaaacatctttcatggtttattgctattcagagatcagcagccaggcatcgcatgaacaaacaagatcaaatcttttctggtcagttattacgagtaagagacagatgtcaaaccattagtcttgttgtcattagctaggcccagatatatgtctgcacacagagccagagca

SEQ ID NO: 380 - DNA, 7881 bp, Homo sapiens
gctctggcagcactggaggcggcggcggcccgagggcgacttgcggggcgcgcaggccgccgtgcacccgggacgcttccccctcggggaccctccgcgggcttctccgccgcgccgtccggcgggagccggcgggaccccgggcgagcggcgcgggcggcaccatgaggcggcagtggggcgcgctgctgcttggcgccctgctctgcgcacacggcctggccagcagccccgagtgtgcttgtggtcggagccacttcacatgtgcagtgagtgctcttggagagtgtacctgcatccctgcccagtggcagtgtgatggagacaatgactgcggggaccacagcgatgaggatggatgtatactacctacctgttcccctcttgactttcactgtgacaatggcaagtgcatccgccgctcctgggtgtgtgacggggacaacgactgtgaggatgactcggatgagcaggactgtcccccccgggagtgtgaggaggacgagtttccctgccagaatggctactgcatccggagtctgtggcactgcgatggtgacaatgactgtggcgacaacagcgatgagcagtgtgacatgcgcaagtgctccgacaaggagttccgctgtagtgacggaagctgcattgctgagcattggtactgcgacggtgacaccgactgcaaagatggctccgatgaggagaactgtccctcagcagtgccagcgcccccctgcaacctggaggagttccagtgtgcctatggacgctgcatcctcgacatctaccactgcgatggcgacgatgactgtggagactggtcagacgagtctgactgctcctcccaccagccctgccgctctggggagttcatgtgtgacagtggcctgtgcatcaatgcaggctggcgctgcgatggtgacgcggactgtgatgaccagtctgatgagcgcaactgcaccacctccatgtgtacggcagaacagttccgctgtcactcaggccgctgtgtccgcctgtcctggcgctgtgatggggaggacgactgtgcagacaacagcgatgaagagaactgtgagaatacaggaagcccccaatgtgccttggaccagttcctgtgttggaatgggcgctgcattgggcagaggaagctgtgcaacggggtcaacgactgtggtgacaacagcgacgaaagcccacagcagaattgccggccccggacgggtgaggagaactgcaatgttaacaacggtggctgtgcccagaagtgccagatggtgcggggggcagtgcagtgtacctgccacacaggctaccggctcacagaggatgggcacacgtgccaagatgtgaatgaatgtgccgaggaggggtattgcagccagggctgcaccaacagcgaaggggctttccaatgctggtgtgaaacaggctatgaactacggcccgaccggcgcagctgcaaggctctggggccagagcctgtgctgctgttcgccaatcgcatcgacatccggcaggtgctgccacaccgctctgagtacacactgctgcttaacaacctggagaatgccattgcccttgatttccaccaccgccgcgagcttgtcttctggtcagatgtcaccctggaccggatcctccgtgccaacctcaacggcagcaacgtggaggaggttgtgtctactgggctggagagcccagggggcctggctgtggattgggtccatgacaaactctactggaccgactcaggcacctcgaggattgaggtggccaatctggacggggcccaccggaaggtgttgctgtggcagaacctggagaagccccgggccattgccttgcatcccatggagggtaccatttactggacagactggggcaacaccccccgtattgaggcctccagcatggatggctctggacgccgcatcattgccgatacccatctcttctggcccaatggcctcaccatcgactatgccgggcgccgtatgtactgggtggatgctaagcaccatgtcatcgagagggccaatctggatgggagtcaccgtaaggctgtcattagccagggcctcccgcatcccttcgccatcacagtgtttgaagacagcctgtactggacagactggcacaccaagagcatcaatagcgctaacaaatttacggggaagaaccaggaaatcattcgcaacaaactccacttccctatggacatccacaccttgcacccccagcgccaacctgcagggaaaaaccgctgtggggacaacaacggaggctgcacgcacctgtgtctgcccagtggccagaactacacctgtgcctgccccactggcttccgcaagatcagcagccacgcctgtgcccagagtcttgacaagttcctgctttttgcccgaaggatggacatccgtcgaatcagctttgacacagaggacctgtctgatgatgtcatcccactggctgacgtgcgcagtgctgtggcccttgactgggactcccgggatgaccacgtgtactggacagatgtcagcactgataccatcagcagggccaagtgggatggaacaggacaggaggtggtagtggataccagtttggagagcccagctggcctggccattgattgggtcaccaacaaactgtactggacagatgcaggtacagaccggattgaagtagccaacacagatggcagcatgagaacagtactcatctgggagaaccttgatcgtcctcgggacatcgtggtggaacccatgggcgggtacatgtattggactgactggggtgcgagccccaagattgaacgagctggcatggatgcctcaggccgccaagtcattatctcttctaatctgacctggcctaatgggttagctattgattatgggtcccagcgtctatactgggctgacgccggcatgaagacaattgaatttgctggactggatggcagtaagaggaaggtgctgattggaagccagctcccccacccatttgggctgaccctctatggagagcgcatctattggactgactggcagaccaagagcatacagagcgctgaccggctgacagggctggaccgggagactctgcaggagaacctggaaaacctaatggacatccatgtcttccaccgccgccggcccccagtgtctacaccatgtgctatggagaatggcggctgtagccacctgtgtcttaggtccccaaatccaagcggattcagctgtacctgccccacaggcatcaacctgctgtctgatggcaagacctgctcaccaggcatgaacagtttcctcatcttcgccaggaggatagacattcgcatggtctccctggacatcccttattttgctgatgtggtggtaccaatcaacattaccatgaagaacaccattgccgttggagtagacccccaggaaggaaaggtgtactggtctgacagcacactgcacaggatcagtcgtgccaatctggatggctcacagcatgaggacatcatcaccacagggctacagaccacagatgggctcgcggttgatgccattggccggaaagtatactggacagacacgggaacaaaccggattgaagtgggcaacctggacgggtccatgcggaaagtgttggtgtggcagaaccttgacagtccccgggccatcgtactgtaccatgagatggggtttatgtactggacagactggggggagaatgccaagttagagcggtccggaatggatggctcagaccgcgcggtgctcatcaacaacaacctaggatggcccaatggactgactgtggacaaggccagctcccaactgctatgggccgatgcccacaccgagcgaattgaggctgctgacctgaatggtgccaatcggcatacattggtgtcaccggtgcagcacccatatggcctcaccctgctcgactcctatatctactggactgactggcagactcggagcatccaccgtgctgacaagggtactggcagcaatgtcatcctcgtgaggtccaacctgccaggcctcatggacatgcaggctgtggaccgggcacagccactaggttttaacaagtgcggctcgagaaatggcggctgctcccacctctgcttgcctcggccttctggcttctcctgtgcctgccccactggcatccagctgaagggagatgggaagacctgtgatccctctcctgagacctacctgctcttctccagccgtggctccatccggcgtatctcactggacaccagtgaccacaccgatgtgcatgtccctgttcctgagctcaacaatgtcatctccctggactatgacagcgtggatggaaaggtctattacacagatgtgttcctggatgttatcaggcgagcagacctgaacggcagcaacatggagacagtgatcgggcgagggctgaagaccactgacgggctggcagtggactgggtggccaggaacctgtactggacagacacaggtcgaaataccattgaggcgtccaggctggatggttcctgccgcaaagtactgatcaacaatagcctggatgagccccgggccattgctgttttccccaggaaggggtacctcttctggacagactggggccacattgccaagatcgaacgggcaaacttggatggttctgagcggaaggtcctcatcaacacagacctgggttggcccaatggccttaccctggactatgatacccgcaggatctactgggtggatgcgcatctggaccggatcgagagtgctgacctcaatgggaaactgcggcaggtcttggtcggccatgtgtcccacccctttgccctcacacagcaagacaggtggatctactggacagactggcagaccaagtcaatccagcgtgttgacaaatactcaggccggaacaaggagacagtgctggcaaatgtggaaggactcatggatatcatcgtggtttcccctcagcggcagacagggaccaatgcctgtggtgtgaacaatggtggctgcacccacctctgctttgccagagcctcggacttcgtatgtgcctgtcctgacgaacctgatagccagccctgctcccttgtgcctggcctggtaccaccagctcctagggctactggcatgagtgaaaagagcccagtgctacccaacacaccacctaccaccttgtattcttcaaccacccggacccgcacgtctctggaggaggtggaaggaagatgctctgaaagggatgccaggctgggcctctgtgcacgttccaatgacgctgttcctgctgctccaggggaaggacttcatatcagctacgccattggtggactcctcagtattctgctgattttggtggtgattgcagctttgatgctgtacagacacaaaaaatccaagttcactgatcctggaatggggaacctcacctacagcaacccctcctaccgaacatccacacaggaagtgaagattgaagcaatccccaaaccagccatgtacaaccagctgtgctataagaaagagggagggcctgaccataactacaccaaggagaagatcaagatcgtagagggaatctgcctcctgtctggggatgatgctgagtgggatgacctcaagcaactgcgaagctcacgggggggcctcctccgggatcatgtatgcatgaagacagacacggtgtccatccaggccagctctggctccctggatgacacagagacggagcagctgttacaggaagagcagtctgagtgtagcagcgtccatactgcagccactccagaaagacgaggctctctgccagacacgggctggaaacatgaacgcaagctctcctcagagagccaggtctaaatgcccacattctcttccctgcctgcctgttccttctcctttatggacgtctagtccttgtgctcgcttacaccgcaggccccgcttctgtgtgcttgtcctcctcctcctcccaccccataactgttcctaagccttcaccggagctgtttaccacgtgagtccataactacctgtgcacaagaaatgatggcacatcacgagaatttagacctggattttaccatgaacctcacatcttgtactccatcctgggccccctgaaactgcttattcgtgattcctcaccagcgtagagctccacctcccctttccccagtaccctcagtgcctgcttctcagtgctgatgcagctgatgacccaggactgcgctctgccccatcacagccagcatgactgcttctctgagagaacttgcccatcaggggctgggacatgggggtgtgggtaaagacagggatgaaggatagaggctgagagaagaaggaagaatcagcccagcaggtatgggcatctgggaaacctccagcctcaagtgtgttggtaacatgaaaaagctctggggggtagttggatctgggtgtctggtccattgctggcagtggacattattcttgccctaagagacactgccttttcagcagcagatactggtgagatgggggtggctcaggctgttcttcctcctcctagaatgtctggagctgtttctacattcagataactgggtcccctatcacaaggctactggctaataggaattccctcctggtgccaccactggccagtacctttcctaagtctttgctcaaattaaccaggttgtgagccagtggcttgagtgaatgttaggccttgggggctgagtctctgaaaagtctaagaagctctgcctagaccaaatatggtatacctcctgacccctctctccctcatgtcctgggattctggggaagagacctagaaacaagctttcaaagaaaaaccagaagttgtcataaatggtcagaaagaacgatcaggttggagacttgggaaacccagggcctaaagagaagtatccatgagggtcaaacttcctgttgaacttcctatgttctttctcaagtgctcagggatctaagttagtggacagcaagcctgtggctacggggtggtgatgttcctcttccagctgtcccctcagctaaggggcttagtttccatgtgggatgccatcacttggttcatgctcattcacacaaagggcacgtgtctcagcctggtatcagggaaattgagacttatttttgccctaaaacgtctccctagctgttcttcgtggggttttttttgtttgtttttttgcctaatttgctttttctgaccaagccttgtggcaccagcaatctccaaagtcctgtggtgggagggctgaataaataaaaatacaaagaggtgggtaaggagtaggaaggtagagagcaccactgatgaggccctcctagcccatggcagacccagacctcttctcccccaggaattagaagtggcaggagagaacaacaggggctgggaatggaggggagaatttctaggggaagtttcctgagttgaaacttctcctgtggttactggtattgagaaatcagctaccaaagtgaaaaaggacaagatcaattcttttctagtcagttctaagactgctagagagagataccaggcccttagccttgctctcagtagcgtcagccccagttctgagcctccccacattacacttaacaagcagtaaaggagtgagcactttgggtccttagactcatgtctggggaggaagagcaagtagaaaagtggcattttcttgattggaaagggggaaggatcttattgcacttgggctgttcagaatgtagaaaggacatatttgaggaagtatctatttgagcactgatttactctgtaaaaagcaaaatctctctgtcctaaactaatggaagcgattctcccatgctcatg

SEQ ID NO: 381 - DNA, 7784 bp, Rattus norvegicus
cggccgcagcaccatgaggcggtggtggggcgcgctgctgctcggcgccctgctctgcgcacacggcacagccagcaatctggagtgtgcgtgtggtcggagccacttcacatgtgcagtgagcgcgctcggcgaatgtacctgcatccccgcccagtggcagtgtgatggtgacaatgactgtggagaccacagcgatgaggatggctgtacgctgcctacctgttcccctctggacttccactgtgacaacggcaagtgcatccgacgctcctgggtgtgtgatggggacaatgactgtgaagatgactccgatgagcaggactgtcccccccgggagtgtgaggaggacgagttcccctgccagaacggctactgcatccggagtctgtggcactgtgatggggacaatgactgtggtgacaacagtgacgaacagtgcgacatgcgcaagtgctcagacaaggaattccgctgcagtgatgggagctgcattgctgagcattggtactgtgatggagacacagactgcaaagacggctctgatgaggaaagctgcccctcagcagtaccctctcctccttgcaacctggaagagttccagtgtgcctacggccgccgcatccttgacatctaccactgtgacggggatgatgactgcggagactggtcagacgagtccgactgctcctcccaccagccctgccgctctggggaattcatgtgtgacagtggcctgtgcgtcaacgcaggctggcgctgtgatggtgatgcggactgcgatgaccagtctgatgaacgtaactgcaccacctccatgtgcacagctgaacagtttcgctgtcggtcaggccgctgtgtccgtctgtcctggcgctgtgatggggaggatgactgcgcggacaacagtgatgaagaaaactgtgagaacacaggaagcccccaatgcgcctctgaccagttcctgtgttggaatgggcgttgtattgggcagaggaagctctgcaatggggtcaatgactgcggcgacaacagtgacgaaagcccacagcagaactgtcggccccggacgggtgaggagaactgcaatgttaacaacggtggctgtgcccagaagtgtcagatgattcgaggggcagtgcagtgtacctgccacacaggctaccggctcacagaggatgggagaacgtgccaggatgtgaatgaatgtgctgaggagggatactgcagccagggctgcacaaacagcgaaggggctttccagtgctggtgcgaagccggttatgagctgcggcccgatcggcgcagctgcaaggctctggggccggaacctgtgctactgtttgccaatcgcatcgacatccggcaggtgctcccgcaccgctccgaatacacgctgctactgaacaacctggagaacgccattgcccttgactttcatcatcggcgggagctggtcttctggtctgatgtcaccctggaccgcatcctccgtgccaaccttaatggcagcaatgtggaggaggtggtgtctactgggctagagagcccaggcggcttggctgtggattgggtccatgacaagctctactggactgactccgggacgtcgaggattgaggtggctaatctggacggggcccaccggaaggtgctgctgtggcagagcctggagaagcccagggccattgccctgcaccccatggagggaaccatctattggacagactggggcaacaccccccgcatcgaggcctccagcatggacggctctggacgtcgcatcattgctgacactcatcttttctggcccaacggcctcaccatcgactacgctggacgccgtatgtactgggtggacgctaagcaccacgtcatcgagagagccaatctagatgggagtcaccgcaaggctgtcattagccagggcctcccacatccctttgccatcacagtgtttgaagacagcttgtattggacagactggcacaccaagagcatcaatagtgccaacaagttcactgggaagaaccaggagatcattcgcaacaagctccactttcctatggacattcataccttgcacccccagcgccagcctgcagggaaaaaccgctgcggggacaacaacggaggttgcactcacctgtgtctacccagtggccagaactacacttgtgcctgccccaccggcttccgaaaaatcaacagccacgcctgtgcccagagtcttgacaagttcctgctttttgcccgaaggatggacatccgtcggatcagcttcgacacagaggacctgtccgacgatgtcatcccactggctgacgtgcgcagtgctgtggcccttgactgggactcccgggatgaccacgtgtactggacagatgtcagcactgataccatcagcagggccaagtgggatggaacaggacagaaggtggtagttgacaccagtttggagagccctgctggcctggcaattgattgggtcaccaacaaactctactggacagatgcaggtacagaccggattgaagtggccaacacggacggcagcatgaggacagtgctcatctgggagaaccttgatcgtcctcgggacatcgtggtagaacccatgggcgggtacatgtattggactgactggggtgcaagtcccaagattgaacgagctggcatggatgcctccaaccgacaagtcatcatctcttcgaatctgacatggcctaatggattagctattgactatgggtcccagcggctgtactgggctgatgccggcatgaagactattgaatttgctgggctggatggcagcaagaggaaggtactaattggaagtcaactcccccaccctttcgggctgactctatacggacagcgcatctattggactgactggcagaccaaaagtatccaaagtgctgaccggctgactgggctagaccgggagactctgcaggagaacctggagaatcttatggacatccatgtcttccaccgccagaggcctccagtgacgacaccatgtgctgtggagaatggcggctgcagccatttgtgtcttcggtccccaagtccgagcggcttcagctgtacctgccccacaggtatcaacctgctgcttgatggcaagacatgctcaccaggcatgaacagtttcctcatcttcgccaggaggattgatgttcgcatggtctccttggacatcccttattttgctgatgtggtggtacccatcaacatgaccatgaagaacaccattgccattggagtggacccgctggaaggaaaagtgtactggtctgacagcacactgcacaggatcagccgtgccagcctggatggctcacagcacgaggacatcatcactacaggtttgcagaccacagatggacttgcagtggatgccatcggccggaaagtgtattggacagacacgggaacgaatcggattgaagttggcaacctggatgggtctatgcggaaagtgttggtgtggcagaaccttgacagtccccgggccattgtgctataccatgaaatggggttcatgtactggacagactggggggagaatgccaaactagagcgttccgggatggatggctcagaccgtactgtgctcatcaacaacaacctagggtggcctaacggactgactgtggacaagaccagctcccaactgctgtgggctgatgcccatactgagagaattgaggtggctgacctgaacggtgccaatcggcatacattggtgtcaccagtgcagcacccatatggcctcactctgcttgactcttacatctactggactgactggcagacccgtagcatccaccgggccgacaagagtactggcagcaatgttatcctcgtgaggtccaacctgccaggcctcatggacatccaagctgttgaccgggcacagccactgggttttaataagtgtggttcgagaaatggcggctgctcccacctctgtttacctcggccctctggtttctcctgtgcctgccccactggcattcagctgaagggggatgggaagacctgtgacccctctccggagacctacctgctcttctctagccggggctccattcgccgtatctcactggacaccgatgaccacacagacgtgcatgtccctgttcctgggctcaacaatgtcatctccctagactatgacagtgtggatggaaaggtctattacacagacgtgttcctggatgttatcaggcgagcagacctgaatggcagcaacatggagactgttattgggcatgggctgaagaccactgatgggctggcagtggactgggttgccagaaacctgtactggacagatacaggccgaaataccatcgaagcatctcggctagatggttcctgtcgcaaagtgctaatcaacaacagtctggacgagccccgggccattgctgttttccccagaaaggggtacctcttctggacagactggggccacattgccaagattgaacgggcaaacctggatggttctgagaggaaggtcctcatcaatgcagacctaggctggcccaatggccttaccttggactatgatacccgcaggatctactgggtagatgcacatctggatcggattgagagtgctgacctcaatgggaagctgaggcaggtcttggtcagccatgtgtcccacccctttgccctcacacagcaggacaggtggatctactggacagactggcagaccaaatccatccagcgtgtcgacaagtactcaggccgcaacaaggagacagtgctggccaatgtggagggactcatggatatcatcgtggtgtcccctcaacggcagacagggaccaatgcctgtggtgtgaacaatggtggctgtagccacctctgctttgccagagcctctgactttgtgtgcgcctgtcctgatgagccagacagtcatccctgctccctcgtgcctggcctcatgcctcctgctcccagagctaccagcctgaatgaaaagagcccagtgctccccaacacattgcccaccaccttacattcttctaccaccaggacccgcacatctccagagggggcagaaggaagatgctctgaaagggacgcccagctgggcctctgtgcacattccaatgaagctgtacctgctgctccaggtgaaggactgcatgtcagctacgccgttggtggactcctcagcgtcctgctgattttgttggtgactgcagctttgatgctatacagacacagaaaatccaaattcacggatcctggaatgggaaacctgacctacagcaacccctcctaccgaacttccactcaggaagtgaaaattgaagcagctcccaaaccagccatgtataatcagctgtgctataagaaagagggtggacctgaccacagctacaccaaggagaagatcaagattgtagagggaattcacctcctggctgggcatgatgctgaatggggtgacctcaagcaactgcgcagctccagggggggccttctacgggatcacgtatgcatgaagacagacacggtgtccatccaggccagctctggctccctagatgacacggagacagagcagctgctgcaggaagagcagtctgagtgtagcagtgttcacactgcaaccactccagagagacggggctctctgccagacacaggctggaaacatgaacgcaagctctcctcagaaagccaggtctaagtgcccacattctcttccctgcctgcctgttcgttcttctctgtggacttctgatccttgtgctcacaaagcaacgggccctttcccgtgcactcatcttcctcctcccacccaaaagtgttcccacgaccccatagaacctgttactcacctgagtctgaaactacctggggacaagaagtgagggcacatctcaagaatttagacctggattttaccatgaacttcacctcttgcactctatcctgggccccctgaagctgggctcagtgtaataccttgtcagcacagcatcttcctttgccctggcatcctatgtgtctgccccacggcaccgatgcaattggtgatgtaagactttggtcttccccatcacagctgacatgatggcctctctgtgaactggcctaccaggggctgggactggaggtcacaggtaaaaggtaatgaatggatagaggttgagagaaggaagaaccagcccagctggtctgggtgtttgggaaacctccagcctcaagtgttggtaattatgaaaaacctttgggggaaggcacttgagccctgggatctcgtccattgctggcagtggacgttctcacccaggtgaagtgtgggtatggcctaagttcttcttcctcctgctggaatgtctggggccactatctaccttcagtaactggaggcctctgtcatggcaactcactaataggaattccctcctggtgctgctactggccagtacttttcttaagcctttaactatgctgtgagcttgagtgaacattggtcgggccttggagagtaggcctctagaacgtttaagaagctctgcaaagatcaagaacagtatcttacctgagcctcactactctgcccttttttctcccattctctgggattctaagggaagcaattctatcacagcctttcaaagaatggctagacattttcctaaatggtcaggaagagcaactagagagacacaagacacccaggggctcaggagaagtatcgtgaggttgaacttcttgagttcattctcaggtgctcagggacccaagcctggggcactaaggtcatgtgccaactcttctcggcctctgcccacactaaagtgccaccattagctgcatgcttctttaggccaagggcttctctcaggggagctgagagtctttttgtgctgccctagaatttcatcctagtgtttcacttctgagagaggggaatctagacttgttttctctaaccgatcctgtatcaccttggtctccattgtccagtgatgagtgaggaggttcaatgaagatgcaaagtggtgagcagggagtggagaggtggtgagattaggtccagagcaagtctaggcacttctccatagaaactgaagcacaggagagattgacaggagctgaacatggaggggagggtttttaggggaagttttctgacttaaaacatctttcatggttattgctacacagagatcagcagccaggcatatgcatgaaaaacaggacaagatcaaatcttttctggtcagttattaggagtaggagacagatgtcaaaccattagtctcgttgtcactagctaggcccagacctatgtctacacatagagaaccaaggcaggccaaaggccttttccagtacaccaactgagaaaagaacagtgccatttttaagaggaaagacagacatttcacacagaatgaggcaacatttgagcctttaaacccgtagctggggaggaggaagaaaacaggagtgtggtgtcctcctgagtggaaagggcaaggttcttattgcactcgggctgttcagaatgtagaaaggacgcatttgagggaatatctatttgagcactgatttactctgtaaagagggcaaaatctctctgtcctaaactaatggaagtgattttcccatgctcatgtgtaatgattttaacattactcactggagagattggactttctggagttatttaaccactatgttcagtattttagggctttatgataatttaatataaatttagctttctt

SEQ ID NO: 382 - DNA, 5835 bp, Gallus gallus
atgtggctaatggaagacctgactgtgactcgcagtccgcttgaacttggcctgtgtcagcactccctgcacctccatgccgagggagtgctgaacggagcggatccaacgctgggatcaaagctggaaacaaaaggctttgggagaaaaatttcccgtctggccaagaggcagccacacaaaggcataacaagtagtaccgaatgctcctgtggacgcaatcactttacgtgtgcagtcagtgcttttggggaatgtacatgcatcccggctcagtggcagtgtgatggagacaatgactgtggggaccacagcgacgaagatggctgcatgctgcccacttgctcccccttggacttccactgtgacaacgggaagtgcatccgccggtcgtgggtctgcgatggagacaacgactgtgaggatgattctgatgaacaggactgcccgccacgagaatgtgaggaagatgagttctcgtgtcagaacgggtattgcatccgcagcctgtggcactgcgatggtgacaatgactgtggagacaacagtgacgagcagtgtgatatgaggaaatgctcagagaaggagttccgttgcagcgatggcagctgcatagctgagcactggttctgtgatggtgacacagactgcaaagatggttcggatgaagagaattgcccatcagatgttccagctgccacctgcagcttggaggaattccagtgtgcatatggacggtgcatcttggacatctaccactgtgatggcgacgatgactgtggggactggtctgatgaatctgactgctcatctcaccagccatgtcgctccggggagttcatgtgcaacagtggcttgtgtattaatgctggttggcggtgtgatggagactctgactgtgatgaccagtctgatgagaggaactgcactacatctatgtgtacagctgaccagttccgctgtaaatcagggcgctgtgtccgtttgtcctggcgttgtgatggggaagatgactgctctgacaacagtgatgaggagaattgtgaaaatacagggacccctcagtgtgctccagaccagttcctgtgtgggaatgggcgttgcattggccaacggaagctctgcaatggtgcaaatgattgtggagatggcagcgatgagagcccgcatcaaaactgccgtccacgaacaggggaggagaactgcaatgtcaacaatggtggctgtgctcagaagtgccagatggtacgagggatggtgcagtgcacctgccatacaggttacaggcttctggaagatggccggtcatgccaagatgtgaatgaatgtgctgaggaaggttactgcagccaaggctgtaccaacagtgaaggtggtttccagtgctggtgtgagcaaggctatgagctgcgccctgacaagcgtagctgcaaagctctagggccagagccagtgctgctctttgccaatcgaattgatatccggcaagtgttaccacatcgctcagagtatacactgctcctgaacaacttggaaaatgccattgcccttgacttccaccacagcaaggagctggtattttggtctgatgtcactctggaccgcataatgagggccaatctgaatggtagcaacgtggaagaagtggtttctacaggtctggagagcccaggtggacttgctattgactggatacatgacaaattgtactggacagactctggaacatctcgaattgaagtagcaaacttggatggtactcacaggaaggtgcttttgtggcagaacctggagaaaccccgggcgattgccctccatcctatggagggcacaatctactggactgactggggcaacactccccgcattgagtattccaacatggatggctctaatcggcgcatcattgcggatacgcacctattctggcccaatgggctgaccattgactatgcagggcatcggatgtactgggtggatgccaaacaccatgtcatcgagagggctgaccttgatggacggaataggaaagctgttattagccaaggccttccacacccatttgctatcaccgtgtttgaggacagtttgtactggacagactggcacaccaagagcattaacagtgccaacaaattcacaggcaagaaccaggagatcatccgaaacaaactccacttccccatggacatccacacactgcatcctcagcgtcagccagcagggagaaaccgctgtggggccaacaatgggggctgtacacacctgtgcctgccgagcagcaaggattatacctgcgcctgccccactggcttccgcaagaccagcagccatgcctgtgctcagagtcttgacaagttcctgctttttgcccgaaggatggacatccgtcggatcagctttgacacagatgacctgtcagatgatgtcatcccccttgctgatgtgcgcagcgcagtggctctggactgggactcaaaggatgactatgtctactggacagatgttagcaccgactcaatcagccgagcaaaatgggatggatcaggacaggaggttgtggtggataccagcctggaaagtccagctggactggcaattgactgggtcaccaacaaactatactggactgatgcaggaacagatcgaatagaagtctccaacacagatgggaccatgaggactgttctgatctgggagaacctagacagacctagagatattgtagtggaccctgttggagggttcatgtactggactgactggggagccaacccaaaaattgagcgtgctggtatggatgcttcaaatcgcttggtgatcatttcctccaacttgacgtggcctaatggcttggccattgactatgagtcacagcgcttgtactgggcagatgcaggcatgaagaccattgagtatgccagtctggatggcagcaacaggaaggtgctgattgggagcaatctacctcacccatttggacttactctttatggcgagagaatctactggacagactggcaggccaaaagcatccagagcgcagatagaaggactgggcaggcacgggaaacgctacaggacaatctggagaatcttatggatattcatgttttccaccggcataggccaccagtacgtacaccatgtgaagttaacaatggaggctgcagtcacctgtgccttctggcacctcttccgaaaggttatagctgtacctgtcctactgggatcaacctacagtctgatggcaagacgtgctccactggaatgaccagctttctgatctttgccagaaggactgacatacggatggtctctctggatattccctactttgcagatgtcgtcgtttcagtcaatgtcaccatgaaaaacaccattgcaattggagtggatcctcatgaaggaaaggtttactggtcagacagcacactgcgaaaaataagccgggctgcccttgatggctccaaatttgaggacatcatcactacaggtctgttgactacagatgggctggccgtggatgctattggcagaaagatatattggacagatacaggaacaaaccgtattgaagtgggaaatcttgatggctctatgaggaaagtcttggtctggcagaatctggacagcccacgggcaatagccttataccatgagatggggtatatgtactggacagactggggtgagaatgccaagctggaacgctctgggatggatggctcaggacgtgtcgttttgatcagcaacaacctgggctggcccaatggactggcagtggataaggctgggtcccagctgctctgggctgatgcacacactgagcgtattgaggctgcagatctcaatggtgctaaccgccgcaccctgctgtccccagtgcaacacccctatggcctcactttgctggactcttacatctattggaccgactggcaaactcgcagcattcacagggctgacaaggacagcggtgccaatgtcatcctggtgagggcaaacctgcctggccttatggacattcaagctgttgatagagcacggcccctaggctttaataaatgtggagttcgtaacggtggctgctcccacctttgcttgcctcaccccactggattctcctgtgcctgccccactggcatccagctgaagcgagatgagcagacatgtgactcttctccagagacctacctacttttctctagccgtgcttccattcgtcgtatctcgctggataccagtgaccacacagatgtgcacatccctgtccctgagctgaacaatgtcatttctctggattatgacagtgtggatggcaagatttactacacagatgtttttcttgatgtcatcaggcggtctgatctcaatggcagcaacatggaaactgttattggtcagggcctgaagactacagatggtctggcagtggactgggttgctaggaacctctactggacagacacaggacgcaatactgttgaagtagctagactggatgggagctccaggaaagtgctgatcaacaacagtctggatgagccccgagccattgctgtctttcccaagaaggggtacctcttctggacagattggggtcacattgctaaaattgaacgggcaaacttggatggctctgaacgtaaaatcctgatcaacactgacctaggatggccaaatggcttgaccttggattatgacaccagaaggatatactgggtggacgcgcatcttgatcgcatagagagttgtgatctcaatgggaagctacgacaagtgctggtcagccaggtgtcacatccctttgctctgactcagcaggataggtggatatattggactgactggcaaacaaaatctatacagagagtggacaaatactctgggcggaacaaagagacagtcctggccaatgttgaggggctgatggatatcattgtggtttcccctcaaagacagacaggtactaatgcttgtggagtgaacaacggaggctgcactcacctttgctttgccagagcttctgactttgtctgtgcatgcccagatgaaccagatgggcggccctgttctactgttcctggcgtggtaccctttggtcctgaaatcaccagtgagagaagtcagacaccacctggtagaataggtacctcaacaaccaagcctcttacatctctggaagaagaggaaggaaactgctcggacaaagatgctaggcgcggcttgtgtactcatgccaacgaggcagtgttggcaacaatgggtgagggactgcatgttagctacatcattggaggccttctcagcgtcttgttcattttgctgcttattgcttctttaatcatatataggcataataaatccaagttcacggaccctggcctagggaacctaacctatagtaacccctcgtatcggacatctacccaggaggtgaagattgaaacaattcccaagccaacaatgtacaaccaattatgctataaaaaagagactggtcctgatcacagctacaccaaggagaagataaagattgtggaagggatatgccttttatccagtgatgattcagagtgggatgaccttaagcaaattcggagctctcgaggagggatcctccgggaccatgtttgcatgaagacagacacagtctctatccaggctagttctggttcactggatgacactgaaactgagcagctgctacaggaagaacaatctgagtgcagcagtgttaacacagcagcggccactcctgaacggcgtggctcactcccagacacaggctggaaacaccaacgcaagccctccacggagagcgaggtctaa

SEQ ID NO: 383 - DNA, 5166 bp, Mus musculus
gcccgaggcgggagcaagaggcgccgggagccgcgaggatccaccgccgccgcgcgcgccatggagcccgagtgagcgcgcggcgctcccggccgccggacgacatggaaacggcgccgacccgggcccctccgccgccgccgccgccgctgctgctgctggtgctgtactgcagcttggtccccgccgcggcctcaccgctcctgttgtttgccaaccgccgggatgtgcggctagtggatgccggcggagtgaagctggagtccaccattgtggccagtggcctggaggatgcagctgctgtagacttccagttctccaagggtgctgtgtactggacagatgtgagcgaggaggccatcaaacagacctacctgaaccagactggagctgctgcacagaacattgtcatctcgggcctcgtgtcacctgatggcctggcctgtgactgggttggcaagaagctgtactggacggactccgagaccaaccgcattgaggttgccaacctcaatgggacgtcccgtaaggttctcttctggcaggacctggaccagccaagggccattgccctggatcctgcacatgggtacatgtactggactgactggggggaagcaccccggatcgagcgggcagggatggatggcagtacccggaagatcattgtagactccgacatttactggcccaatgggctgaccatcgacctggaggaacagaagctgtactgggccgatgccaagctcagcttcatccaccgtgccaacctggacggctccttccggcagaaggtggtggagggcagcctcactcacccttttgccctgacactctctggggacacactctactggacagactggcagacccgctccatccacgcctgcaacaagtggacaggggagcagaggaaggagatccttagtgctctgtactcacccatggacatccaagtgctgagccaggagcggcagcctcccttccacacaccatgcgaggaggacaacggtggctgttcccacctgtgcctgctgtccccgagggagcctttctactcctgtgcctgccccactggtgtgcagttgcaggacaatggcaagacgtgcaagacaggggctgaggaagtgctgctgctggctcggaggacagacctgaggaggatctctctggacacccctgacttcacagacatagtgctgcaggtgggcgacatccggcatgccattgccattgactacgatcccctggagggctacgtgtactggaccgacgatgaggtgcgggctatccgcagggcgtacctagatggctcaggtgcgcagacacttgtgaacactgagatcaatgaccccgatggcattgctgtggactgggtcgcccggaacctctactggacagatacaggcactgacagaattgaggtgactcgcctcaacggcacctcccgaaagatcctggtatctgaggacctggacgaaccgcgagccattgtgttgcaccctgtgatgggcctcatgtactggacagactggggggagaaccccaaaatcgaatgcgccaacctagatgggagagatcggcatgtcctggtgaacacctcccttgggtggcccaatggactggccctggacctgcaggagggcaagctgtactggggggatgccaaaactgataaaatcgaggtgatcaacatagacgggacaaagcggaagaccctgcttgaggacaagctcccacacatttttgggttcacactgctgggggacttcatctactggactgactggcagagacgcagtattgaaagggtccacaaggtcaaggccagtcgggatgtcatcattgatcaactccccgacctgatgggactcaaagccgtgaatgtggccaaggttgtcggaaccaacccatgtgcggatggaaatggagggtgcagccatctgtgcttcttcaccccacgtgccaccaagtgtggctgccccattggcctggagctgttgagtgacatgaagacctgcataatccctgaggccttcctggtattcaccagcagagccaccatccacaggatctccctggagactaacaacaacgatgtggctatcccactcacgggtgtcaaagaggcctctgcactggactttgatgtgtccaacaatcacatctactggactgatgtcagcctcaagacgatcagccgagccttcatgaatgggagctcagtggagcacgtgattgagtttggcctcgactaccctgaaggaatggctgtggactggatgggcaagaacctctattgggcggacacagggaccaacaggattgaggtggcccggctggatgggcagttccggcaggtgcttgtgtggagagaccttgacaaccccaggtctctggctctggatcctactaaaggctacatctactggactgagtggggtggcaagccaaggattgtgcgggccttcatggatgggaccaattgtatgacactggtagacaaggtgggccgggccaacgacctcaccattgattatgccgaccagcgactgtactggactgacctggacaccaacatgattgagtcttccaacatgctgggtcaggagcgcatggtgatagctgacgatctgccctacccgtttggcctgactcaatatagcgattacatctactggactgactggaacctgcatagcattgaacgggcggacaagaccagtgggcggaaccgcaccctcatccagggtcacctggacttcgtcatggacatcctggtgttccactcctcccgtcaggatggcctcaacgactgcgtgcacagcaatggccagtgtgggcagctgtgcctcgccatccccggaggccaccgctgtggctgtgcttcacactacacgctggaccccagcagccgcaactgcagcccgccctccaccttcttgctgttcagccagaaatttgccatcagccggatgatccccgatgaccagctcagcccggaccttgtcctaccccttcatgggctgaggaacgtcaaagccatcaactatgacccgctggacaagttcatctactgggtggacgggcgccagaacatcaagagggccaaggacgacggtacccagccctccatgctgacctctcccagccaaagcctgagcccagacagacagccacacgacctcagcattgacatctacagccggacactgttctggacctgtgaggccaccaacactatcaatgtccaccggctggatggggatgccatgggagtggtgcttcgaggggaccgtgacaagccaagggccattgctgtcaatgctgagcgagggtacatgtactttaccaacatgcaggaccatgctgccaagatcgagcgagcctccctggatggcacagagcgggaggtcctcttcaccacaggcctcatccgtcccgtggcccttgtggtggacaatgctctgggcaagctcttctgggtggatgccgacctaaagcgaatcgaaagctgtgacctctctggggccaaccgcctgaccctggaagatgccaacatcgtacagccagtaggtctgacagtgctgggcaggcacctctactggatcgaccgccagcagcagatgatcgagcgcgtggagaagaccactggggacaagcggactagggttcagggccgtgtcacccacctgacaggcatccatgccgtggaggaagtcagcctggaggagttctcagcccatccttgtgcccgagacaatggcggctgctcccacatctgtatcgccaagggtgatggaacaccgcgctgctcgtgccctgtccacctggtgctcctgcagaacctgctgacttgtggtgagcctcctacctgctcccctgatcagtttgcatgtaccactggtgagatcgactgcatccccggagcctggcgctgtgacggcttccctgagtgtgctgaccagagtgatgaagaaggctgcccagtgtgctccgcctctcagttcccctgcgctcgaggccagtgtgtggacctgcggttacgctgcgacggtgaggccgactgccaggatcgctctgatgaagctaactgcgatgctgtctgtctgcccaatcagttccggtgcaccagcggccagtgtgtcctcatcaagcaacagtgtgactccttccccgactgtgctgatgggtctgatgagctcatgtgtgaaatcaacaagccaccctctgatgacatcccagcccacagcagtgccattgggcccgtcattggtatcatcctctccctcttcgtcatgggcggggtctactttgtctgccagcgtgtgatgtgccagcgctacacaggggccagtgggccctttccccacgagtatgttggtggagcccctcatgtgcctctcaacttcatagccccaggtggctcacagcacggtcccttcccaggcatcccgtgcagcaagtccgtgatgagctccatgagcctggtgggggggcgcggcagcgtgcccctctatgaccggaatcacgtcactggggcctcatccagcagctcgtccagcacaaaggccacactatatccgccgatcctgaacccacccccgtccccggccacagacccctctctctacaacgtggacgtgttttattcttcaggcagcccggccaccgctagaccatacaggccctacgtcattcgaggtatggcacccccaacaacaccgtgcagcacagatgtgtgtgacagtgactacagcaccagtcgctggaagagcagcaaatactacctggacttgaattcggactcagacccctacccccccccgcccaccccccacagccagtacctatctgcagaggacagctgcccaccctcaccaggcactgagaggagttactgccacctcttcccgcccccaccgtccccctgcacggactcgtcctgacctcggccgtccacccggccctgctgcctccctgtaaatatttttaaatatgaacaaaggaaaaatatattttatgatttaaaaaataaatataattgggatttttaacaagtgagaaatgtgagcggtgaaggggtgggcagggctgggaaaactttgtacagtggagaaaatatttataaacttaattttttaaaacataaaaaaaaaaaaaaaa

SEQ ID NO: 384 - DNA, 5039 bp, Homo sapiens
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggacttcaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtggg

SEQ ID NO: 385 - DNA, 5101 bp, Rattus norvegicus
cgggagccgcgagggtccaccgccgccgcgcgcgccatggagcccgagtgagcgcgcggcgctcccggccgccggacgacatggaaacggcgccgacccgggcccctccgccgccgccgcagccgctgttgctgctgctggcgctgtgctgcagcctagtccccgctgcggcctcaccactcctgttgtttgccaaccgccgggatgtacgactagtggatgctggtggagtgaagctggagtccaccattgtggccagtggtctggaggacgcagctgctgtagacttccagttctccaagggtgctgtgtactggacagatgtgagcgaggaggccatcaaacagacctacctgaaccagactggagctgctgcacagaacattgtcatctctggccttgtatcacctgatggcctggcctgtgactgggttggcaagaagctgtactggacagactcagagaccaaccgcatcgaggtggccaacctcaatgggacgtcccgtaaggttctcttctggcaggacctggaccagccaagggccattgccctggatcctgcacatgggtacatgtactggactgactggggggaagcaccccggatcgagcgggcagggatggatggcagtacccggaagatcattgtagactccgacatttactggcccaatgggctgaccattgatctggaggagcagaagctgtactgggctgatgccaaactcagcttcatccaccgtgccaacctggacggctccttccggcagaaggtggtggagggcagcctcactcacccctttgccctgacactctctggggacacactctactggacagactggcagacccgttctatccacgcctgcaacaagtggacaggggagaaaaggaaggagatcctcagtgctctgtattcacccatggacattcaggtgctgagccaggagcggcagcctcccttccacacgccatgtgaggaaggcaacggcggctgctcccacctgtgcctgctgtccccgagggagcccttctactcctgcgcctgccctactggtgtacagttgcaggacaatggcaagacgtgtaaggcaggggctgaggaagtgctgctgctggcccggaggacagacctgagaaggatctctctggacacccctgacttcacagacatagtgctgcaagtgggcgacattcggcatgccatcgccatcgactatgacccactggagggctacgtgtactggacagatgacgaggtgcgggctatccgcagggcgtacctggatggctcaggtgcgcagacccttgtgaacaccgagatcaatgatcccgatggcattgccgtggactgggtcgcccggaacctctactggacagatacaggcactgacagaattgaggtgactcgcctcaacggcacctcccgaaagatcctggtgtccgaggacctggacgaacctcgcgccattgtgttgcaccctgtgatgggcctcatgtactggacagactggggagagaaccccaaaatcgaatgtgccaacctagatgggcgagatcggcatgtcctggtgaacacctcccttgggtggcccaatggactggccctggacctgcaggagggcaagctgtactggggggatgccaaaactgataaaattgaggtgatcaacatagacgggacaaagcggcagaccctgctagaggacaagctcccgcacatttttgggttcactctgcttggggacttcatctactggacagattggcagcgacgcagtattgagagggtacacaaggtcaaggccagccgggatgtcatcatcgatcaactccccgacctgatgggactcaaagcagtgaacgtggccaaggttgtcggaaccaacccgtgtgcggatggaaatggagggtgcagccatctgtgcttcttcaccccacacgccaccaagtgcggttgccccataggcctggagctgctgagtgacatgaagacctgcataatccctgaagccttcctggtgttcaccagcagagccaccatccacaggatctccctggagactaacaacaatgacgtggccatcccgctcaccggtgtcaaggaggcctctgcactggactttgatgtgtccaacaaccacatctactggactgatgtcagcctcaagactatcagccgagccttcatgaatgggagctcagtggagcacgtgattgagtttggcctcgactaccctgaaggaatggccgtggattggatgggcaagaacctctattgggctgacacggggaccaacaggattgaggtggcccgactggatgggcagttccggcaggtgctggtgtggagagaccttgacaaccccaggtcgctggctctggatcctactaaaggctacatctactggaccgagtggggaggcaagccaaggatcgtgcgggccttcatggatgggaccaattgtatgacactggtagacaaggtgggccgggccaatgacctcaccattgattacgccgaccagcgactgtactggactgacctggacaccaacatgattgagtcctccaacatgctgggtcaggagcgcatggtgatagctgatgatctgccctacccctttggcctgactcaatatagcgattacatctactggaccgactggaacctgcatagcattgaacgggccgacaagaccagcgggcggaaccgtaccctcatccagggtcacctggacttcgtcatggacatcctggtgtttcactcctcccgtcaggatggcctcaacgactgcgtgcacagcaatggccagtgtgggcagctgtgcctcgccatccccggaggccaccgctgcggctgtgcttcccactacacgctggaccccagcagccgcaactgcagcccgccctccaccttcttgctgttcagccagaaatttgccatcagccggatgatccccgatgaccagctcagtccggaccttatcctacccctacacgggctgaggaatgtcaaagccatcaactacgacccgctggacaagttcatctactgggtggatgggcgtcagaacatcaagagggccaaggacgacggtacccagccctccatgctgacctctcccagccaaagcctgagcccagaccgacagccacatgacctcagcattgacatctacagccggacactgttctggacctgtgaggccaccaacaccatcaatgtccaccggctggatggggatgccatgggagtggtgcttcgaggggaccgtgacagaccaagggccattgctgtcaatgctgagcgagggtacatgtactttaccaacatgcaggaccatgctgccaagattgagcgagcctccctggatggcacggagcgtgaggttctcttcaccaccggccttatccgtcctgtggcccttgtggtggacaatgccctgggcaagctcttctgggtggatgcagacctaaagcgcattgaaagctgcgacctctcaggggccaaccgcctgaccctggaagatgccaacatcgtgcagccagtaggtctgacggtgctggggaggcacctctactggatcgaccgccagcagcagatgattgagcgtgtggagaagaccactggggacaagaggactagggttcagggccgtgtcacccacctgacgggcatccatgctgtggaggaagtcagcctggaggagttctcagcccatccttgtgcccgagacaatggcggctgctcccatatctgcatcgccaagggtgatgggacaccgcgctgctcatgcccagtccacctcgtgctcctgcagaacctgctgacttgtggtgagcctcctacctgctcccctgatcagtttgcatgtgccactggtgagattgactgcatccctggagcctggcgctgtgacggcttccctgagtgtgctgaccagagtgatgaggaaggctgcccagtgtgctctgcctctcagttcccctgtgctcgaggccagtgcgtggacctgcgtttacgctgcgatgctgtctgtctccccaatcagttccggtgcgccagtggccagtgcgtcctcatcaagcagcagtgcgattccttccccgactgtgctgatggatctgatgagctcatgtgtgaaatcaacaagccaccttctgatgacgtaccagcccacagcagcgccattgggcctgtcattggcatcatcctgtccctcttcgtcatgggcggggtctactttgtctgccagcgtgtgatgtgccagcgttacacaggggccagtggtccctttccccacgagtatgttggtggaacccctcatgtgcctctcaacttcatagccccaggtggctcacagcacggtcccttcccaggcatcccgtgcagcaagtccatgatgagttctgtgagcctggtgggggggcggggcagcgtgcccctctatgaccggaatcacgtcaccggggcctcatccagcagttcatccagcacaaaggccacactctatccaccgatcctgaacccaccaccgtccccggccacagacccctccctgtacaacatggacatgttttattcttcaaacatcccatccactgccagaccatacaggccctacatcattcgaggtatggcacccccaacaacaccttgcagcacagacgtgtgtgacagcgactacagcaccagtcgctggaagaccagcaagtactacctggacttgaattcggactcagacccctacccgcccccgcccactccccacagccagtacctgtctgcagaggatagctgtccaccctctcctgtcactgagagaagctactgccacctcttcccaccccctccatccccctgcacggactcgtcctgacctcggccgtccaccccggcctcgctgcctccctgtaaatatttttaaatatgaacaaaggaaaaatatattttatgatttaaaaaataaatataattgggatttttaagaagtgagaaatgtgagcggtgaaggggtgggcagggctgggaaaactttgtacagtggagaaaatatttataaacttaattttttaaaacataaaaaaaaaaaaaaaa

SEQ ID NO: 386 - DNA, 5168 bp, Gallus gallus
cccttcccgcctccctccccgccgaaggcagcgccccgagcatggaacctgcctgagacgctcggcgggctccgctccggcagctgctgctgcggcacaacatggaggcggcgccgccgctcccgctgctgctgctgctgcggctcctggccgcctcggtgctgcgctgcgagccgggctcggccggggcatctcccttcctgctttttgccaaccgtcgggatgttcgactcgtggatgctggaggtacaaagctggagtccactgtggtggtcagtggtttagaggatgctgctgcggttgacttccagtattcgcaaggcatcgttttctggacagatgtgagcgaagaagccatcaagcaaacttatattaaccaaactgggaatgtggtgcagaatgtcataatttctggtctggtttccccggatggcctggcgtgtgattggattgggaaaaaactttactggacagattcagaaaccaataggatagaagtcgctaatctgaatggaacatctagaaaagtcctcttctggcaagacctcgatcagcccagggcaatagctttggatccagctcatggatacatgtattggactgactggggagaaacacccagaatagaacgggcaggaatggacagcagcacacggaaaataattgttgattcagatatctattggccaaatggactcacaatagatcttgatgagcagaaactttactgggctgatgcgaaattgagtttcattcatagagcaaatctggatggctcctttaggcaaaaggttgttgaaggaagtcttactcatccatttgcattgacgttgtctggggacactttgtattggacggactggcagacacgttccattcatgcctgtaacaaacggacaggagagaagaggagagaaatattatctgctttgtattctcccatggacatccaagtcttaagtccagaccggcagccctactttcacactccctgtgaagaaaacaatggtggttgctcccatctgtgcctgctatcaccaagagaccccttctatagctgtgcctgtcccactggtgtgcagctggaagatgatggcaggacgtgcaaatcaggtgctgaagaagtgttgctgcttgctaggaggactgacttgagaaggatttccttggacatgccagatttcactgacattattctgcagatagacaatatcaggcatgcaattgccatagactatgatcctgtggaaggctacatctactggactgatgatgatgttcgggcaattcgtcgggcgtaccttgatggctctggagcacagactctggtaaccacagaaatcaaccatccagatggtattgctgtggactgggtggcaaggaacctgtactggactgatactggaacagaccgcattgaagtgacccgtttgaatggaacatcaagaaagatccttatatcagaaaatctggatgaacctcgtgcaattgtgctcaatcctgtgatgggctacatgtattggacagactggggtgaaagtccaaagatagaatgtgcttatttggatggatcagagaggagagttctggtgaatacgtccttgggttggcctaacggcttggcactggatcttgaagaggacaaactttactggggagatgccaaaacagataaaattgaggtcataaatgttgatggaacaatgaggaaaaccctcttagaagataaacttccacatatatttgggttcacgctgctgggtgattacatctactggactgactggcagaggcgaagcattgaaagagttcataagataagggctagcagagacattattattgatcagctgccagacttgatgggccttaaagcaacaagcgttaccaaagtttttggaactaatccatgcgcagagaacaacggaggctgcagccatctgtgtttctttacaccccaggagaccaggtgtgcctgtcccattggccttgagctgttgagtgacatgaagacttgcatcattcctgaagccttcttagttttcacaagcagagcagcaattcataggatttcccttgaaaccaataataacgatgttgctattcctcttactggagtcaaggaggcatctgctctggattttgatgtttctgataacagaatctattggactgatgttagtttgaagaccataagtcgagcttttatgaatgggagctctgttgaacacgtgattgaatttgggctagattaccctgagggaatggctgtagactggatgggaaagaacctctactgggcagatactggaactaatcgaattgaggtagcccgcctggatggacagtataggcaggtccttgtgtggaaggacttggacaacccaaggtctttggctcttgatcctactaaagggtatatgtactggacagaatggggaggtaaacctaggatcgttcgagcatatatggatggaacaaacagcatcactttggtggataaagtgggtcgagccaatgacctcacaattgattatgcagaccaacggctgtattggactgacttagacaccagcatgatcgagtcatcaaatatgctaggacaggaacgagaaatcatagcagatgatctacctcatccatttggattgacccagtacagtgactacatctactggacagactggaatcttcacagtatagaaagagcagacaagaccagtgggaagaacaggacacttattcagggccatctggattttgtgatggatatattggtcttccattcttcacgtcaggatggcttgaatgattgtgtgcaaaataatggtcattgtggtcacttgtgccttgccataccaaatggatttagatgtggctgtgctgctcactataccttggatcccaacagcaggaactgcagttctcctaccagtttcctgttgtttagccagaaatctgccatcagccgaatgatacctgatgatcagcagagtccagatatcatcctgcctatgcatggtctaaggaatgtgaaggctattgattatgatcccttagataaattgatttactgggtagatgggcgtcagaatattataaaaagagccaaagatgatggcactcagccttttactgtcatgagctctccaaaccaaagccagaacccagagaagcagccacatgacctcagcattgatatatacagccataccttgtattggacctgtgaggccacaaattctgtcaacgttcacaggctgaatggcgagtcaatagggatggtgctgcgaggtgaccacgacaagcccagagccattgtagtgaatgcagagcgggggtacatgtatttcaccaacatgcaagagcgagctcccaagattgaacgtgcagcccttgatggcacagagagggaagtgcttttcacaactgggttgatccgaccagtagcactggtgattgacaacaaactcggaaagcttttctgggtggacgcagatctgaagcgcatcgaaagctgtgacttgtcaggtgctaaccgtgtgactctggaggactccaacatccttcagccaatgggcctgactgtgctggggaatcacctgtactggatcgatcggcagcagcagatgattgagagagtggagaaaactaatgggtacaaaaggactagaattcaaggccgaattgctcacctaacaggcattcatgctgtggaggaacttgatatggaggaattctctgcacatccgtgctcccgtgacaatggtgggtgctcacacatctgcattgccaaaggggatgggactccaagatgttcgtgtccagagcaccttgtgctcctgcagaatctcttaacgtgtggagaacctccaacttgttccccagaccagtttacctgtgcaactggagagattgactgtatcccaatggcatggcggtgtgatggattcccagagtgtgatgaccagagcgacgaggacagttgcccaatatgctccgcatctcagttccagtgtgagaaaggacaatgtattgacgcgcatctgaggtgtaatggagaaattgactgccaagataaatctgatgaagtggattgtgatacaatttgtctgctcaatcagttccgatgtgccagtggccagtgcatcctcctgaagcaacagtgtgactcctttccagactgcattgatggctcagatgaactcatgtgcgaaaaatcaaagccatcctcagatgaacctcagccccacagcagtgccattggtcccgtcattggaataatactttccctttttgtcatgggaggaatgtactttgtttgccagcgagtggtgtgtcagcgctacgccggacccaacagcccctttccacatgagtacgtcagtggcacgcctcacgtccctctaaattttattgctccaggaagttctcagcatggcacttttactggcatctcttgtggaaagtctatgattagctccatgagcctcatgggagggagcagtggtgcccccttatatgacagaaaccacgttactggagcctcttctagtagctcatccagcactaaagccactttctacccgcagatcttgaacccgcctccatccccagctactgatcgctccctgtataatgcagagatgttttattcttcaaacattccttcaaccacaagatcttacaggccttaccttatacgagggacagctccacccacaaccccgtgcagcacagatgtttgtgacagtgactatactactagtcgatggaaagccaacaagtactatatagatttaaattcagactcagacccttacccccctccacccacacctcgcagccaatacatgtcggcagaagaaagttgccctccctctcctgccacagaaagaagctattttcacctctacccacctccaccctctccttgtacagactcctcatgacctcaacagaaggaactttccctgtaaatatttttaaatatgaacagattttaaatatatattttatgatttaaagataaaaaggggtgggaattaaaaaggaaaaaaaaaagaaatgtgaataaaaatgggtgggagggatggggctgtgaaaattgtacaaaggaagaatatttataaaattgatttttttttaacttaaaaaaaaaaaaaaaa

SEQ ID NO: 387 - DNA, 5440 bp, Mus musculus
ggcggcggcatgggggcaggcgctggcgtcgcgcgagcccgggccggagcggcgcgctagagccctgacgtgcgcccttttctttcttctcgcggccgcggaaagcatgagcgtgcagacccgccgctgcggcggccgccccggctcctcgcccgcgcgtcccctggccgccgggcgccggtgagggccgcgagcgcgcgcggtggtggggggaagatgggggccgtgctgaggagcctcctggcctgcagcttctgcgtgctgctgagagcggcccctttgttgctttatgcaaacagacgggacttgagattggttgatgctacaaatggcaaagagaatgcaacgattgtagttggaggcttggaggatgcagctgcggtggactttgtgtttggtcatggcttgatatactggagtgatgtcagcgaagaagccattaaacgaacagaatttaacaaaactgaaagtgtacagaatgttgttgtttctggattattgtccccggatgggctggcatgtgattggcttggagaaaaattgtactggacagattctgaaactaatcgtattgaagtttctaatttagatggatctttacgaaaagttttattttggcaagagttggatcaacccagagctattgccttagatccatcaagtgggttcatgtactggacagactggggagaagtgccaaagatagaacgtgctgggatggatggctcaagtcgcttcgttataataaacacagagatttactggccaaacggactgactctggactatcaggagcggaagctttactgggccgatgcaaaacttaatttcatccataaatcaaacctggatggaacaaaccggcaggcagtggttaaaggttcccttccacatccttttgccttgacgttatttgaggacacattgtactggactgactggaatacacactctattttggcttgcaacaaatatactggcgagggtctgcgtgaaattcattctaacatcttctctcccatggatatacatgctttcagccaacagaggcagccaaatgctacaaatccatgtggaattgataatggtggttgttcccatttgtgtttgatgtctccagtcaagcctttttatcagtgtgcttgcccaactggggtcaagctgctggagaatggaaagacctgcaaagatggtgccactgaactattgctgttagcccgacggacagacttgaggcgaatttctttggatacacccgattttactgacattgttctgcagttagaagatatccggcatgccattgccatagactatgaccctgtagaaggctacatatactggacggatgacgaagtgagggctatccgtcgctccttcatagatggatctggcagtcagtttgtggtcacggcccagattgctcatcctgatggtattgctgttgactgggttgcaaggaacctgtactggacagacactggcacggatcgtatagaagtgacaaggctcaatgggaccatgaggaagatcttgatttcagaggacttagaggagccccgggctatcgtgttagatcccatggttgggtacatgtattggacagactggggagaaatcccaaaaatagagcgagctgctctggacggatctgaccgagtagttcttgtcaacacttcccttggttggccaaacggcttagccctggattatgatgaaggcacaatatactggggagatgccaaaacagacaaaattgaggttatgaataccgatggcaccgggaggcgagtgctggtggaagacaagatccctcacatatttgggtttaccttgctgggtgactatgtttactggactgactggcagaggcggagcatcgagagagtacacaaacggagcgcagagagggaggtcatcatagatcagctgccagacctcatgggactgaaggccacaagtgttcacagaatcattggttctaacccctgtgctgaggacaatggaggatgtagccatctttgcctgtacaggcctcaggggcttcgatgcgcctgtcccattggctttgagctcatcagtgacatgaagacatgcattgtccccgaggctttccttctgttctcgaggagagcggatatcagacgcatatctttggaaacaaacaacaacaatgtggccattcctctcactggtgtcaaagaagcctctgctttggattttgatgtcacagacaacaggatttactggactgatatatcactgaagactattagcagagcctttatgaatggcagtgcactggaacatgtggtagagtttggcttagattatccagaaggcatggcagtggactggcttgggaagaacttatactgggcagacacaggaacaaatcgcattgaggtatcgaagttggacggacagcaccgacaggttttggtatggaaagaccttgacagtcctcgagctctggcactggatcctgctgaagggtttatgtattggactgagtggggaggcaagcctaagattgacagggctgctatggatggaagtgaacgcactacattagttccaaatgtaggccgagcaaatggtctcaccatcgactatgctaaaaggcggctttactggacagacctggacactaacctaatagaatcctcagatatgctcggactcaaccgtgaagttatagcagatgacttgcctcatccttttggcttaactcagtaccaagattacatctactggacagactggagccgacgcagcattgaacgtgccaacaaaaccagtggccaaaaccgcaccatcatccagggccatttggactatgtgatggacatcctggtcttccactcttcccggcaggcagggtggaatgagtgtgcctccagcaacgggcactgctcccacctctgcttggctgtgcccgtcggaggttttgtgtgtggatgccctgcccactactccctgaatgctgacaacaggacctgcagtgctcccaccaccttcctgctcttcagtcagaagagcgccatcaaccgcatggtgattgatgaacaacagagccctgacatcatccttcctatccacagccttcggaacgtccgggccattgactatgaccctttggacaagcagctctactggattgactctcgacaaaactccatacgaaaggcacatgaagatggtggccagggttttaatgtagttgcaaactcagtcgcaaatcagaaccttgaaatacagccctatgatctcagcattgatatttatagccgttacatctactggacctgtgaagctaccaatgtcattgatgtgacgagattagatggacgatcagttggagtggttctaaaaggcgagcaagacagacctcgagccattgtggtaaaccccgagaaagggtatatgtattttaccaatcttcaggaaagatctcctaaaattgaacgggctgcattggatggtacagaacgagaggtcctctttttcagtggcttaagtaaaccaattgctttggctcttgatagcaagctgggcaagctcttctgggctgactcagatctccggcgaattgaaagcagtgatctctcaggtgccaacaggatcgtgctagaagactctaatatattacagcctgtgggcctgaccgtgtttgaaaactggctctattggattgataaacagcagcagatgattgaaaaaattgacatgactggtcgagaaggaagaaccaaggtccaggctcgaattgctcagctgagtgacatccatgcagtaaaggagctgaaccttcaggagtacagacagcacccttgtgcccaggataatggtggctgttcacatatctgccttgtaaaaggagatggtacgacaagatgctcctgccccatgcacttagttctgcttcaggatgagctgtcctgtggagagcctccaacgtgttctcctcagcagtttacctgcttcactggggacattgactgcatccctgtggcttggcggtgtgatgggttcactgagtgcgaagaccacagcgatgaactcaattgtcccgtgtgctcagagtctcagttccagtgtgccagcgggcagtgcattgatggtgcccttcgatgcaatggcgatgcgaactgccaggacaaatcagatgagaagaactgtgaagtgctttgtttaattgatcagttccgctgtgccaatggtcagtgcgttggaaagcacaagaaatgtgaccacagtgtggactgcagtgacagatctgacgagctggactgttatccaactgaggagccagcaccacaagccaccaacacagttggttctgttattggagtaattgtcaccatttttgtgtctggaaccatatactttatctgccagaggatgctgtgtcctcgtatgaagggagacggggagaccatgactaacgactatgtggttcacagcccggcgtctgtgccccttggttatgttcctcacccaagctctctctctggatctcttccaggaatgtctcgaggcaaatcaatgatcagttccctcagtatcatggggggaagcagtgggcccccctatgatcgagcgcacgtcacgggagcctcctcaagcagttcttccagtaccaaaggcacttacttccctgcaattttgaacccaccaccatcgcctgccacagaaagatcccattataccatggaatttggttattcttccaacagtccttccacacataggtcctacagctataggccgtacagctaccggcactttgcaccgcccaccacaccctgcagcactgatgtctgtgacagtgactatgctcctagccggaggatgacctcggtggcaacagccaagggctacaccagtgacgtgaactatgactcagaacctgtgcccccaccgcccacaccccgaagccagtacttgtcagcggaggagaactatgaaagctgccccccttccccatacacggagaggagttactcccaccacctctacccgccaccaccctccccctgcacggactcctcctgaggagggcccctcctcctctgactgcctccaccgggacatgtaaatacacatctggttgagatctggagggggggagggagctatagagaagagtgaggcagactctgtacagttaacattataaagtgggtgggtgctggagatatttgtacagaagaaaagaatatttatatattttcttaaaacagcagttttgctgcttgtgccataaaagtttgtataaaaaataaatttgtactaaaagttttatttttgcaaactaaatacacagagcatgccttaagcccagtgaagagactgagtacaaaggaaacaggaaaagaaaggcatcactgaccaggagtgtctgggttttaacgataccaaaaaaaaaaaaaaaaaa

SEQ ID NO: 388 - DNA, 4774 bp, Homo sapiens
tcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacg

SEQ ID NO: 389 - DNA, 5092 bp, Rattus norvegicus
cggcccctttgttgctttatgcaaacagacgggacttgagattggttgatgctacaaatggcaaagagaatgcgacgattgtagttggaggcttggaggatgcagctgcggtggactttgtgtttggtcatggcttgatatactggagtgacgtcagcgaagaagccattaaacgaacagaatttaacaaaactgagagtgtacagaatgtcgttgtttctggattattgtccccggatgggctggcatgtgattggcttggagaaaaattgtactggacagattctgaaactaatcgtattgaagtttctaatttagatggatctttacgaaaagttttattttggcaagagttggatcaacccagagctattgccttagatccatcaagtgggttcatgtactggacagactggggagaagtgccaaagatagaacgtgctggaatggatggttcaagtcgctttgttataataaacactgagatttactggccaaatggactgactctggattatgaggagcggaagctttattgggcagatgcaaaacttaatttcatccataaatcaaacctggacggaacaaatcgacaggcagtggttaaaggttcccttccacatccttttgccttgacgttatttgaggacacattgtactggactgactggaatacacactctattttggcttgcaacaagtatactggcgagggtctgcgtgaaattcattctaacatcttctctcccatggatatacatgctttcagccaacagaggcagccaaatgctacaaatccatgtggaattgataatggtggttgttcccatttgtgtttgatgtctccagtcaagcctttttatcagtgtgcttgtccaactggggtcaagcttctggagagtggaaaaacctgcaaagatggtgccactgaactattgctgttagcccgacggacagacttgaggcgcatctctttggatacaccagatttcaccgacattgtcctacagttagaagacatccgacatgccattgccatagactacgaccccgtggaaggctacgtctactggacggacgatgaagtacgggccatccgtcgctccttcattgatggatctggcagtcagtttgtggtcacggcccagattgcccatcctgatggtattgctgttgactgggtggcaagaaacctgtactggacagacactggcactgatcggatagaagtgacaaggctcaatgggaccatgaggaagatcctgatttcagaggacttagaggagccccgggctattgtgttagatcccatggctgggtacatgtactggacggactggggagaaatcccaaaaatcgagcgagctgctctggacggatctgaccgagtagttcttgttaacacttcccttggttggccaaatggcttagccctggattatgatgaaggcacaatatactggggagatgccaaaacagacaaaattgaggttatgaatactgatggcactgggaggcgagtgctggtggaagacaagatccctcacatatttgggtttaccttgctgggcgactatgtttactggactgactggcagaggcggagcatcgagagagtgcacaagcggagtgcagagagggaggtcataatagaccagctgccagacctcatgggactgaaggccacaagcgttcacagagtcattggttctaacccctgtgctgaggagaatggaggatgtagccatctttgcctctacaggcctcagggacttcgatgtgcctgtcccattggctttgagctcattagtgacatgaagacatgcattgttcctgaggctttccttctgttctcacggagagcagatataagacgcatctctttggaaacaaacaacaacaatgtggccattcctctcacgggtgtcaaagaagcttctgctttggattttgatgtcacagacaacagaatttactggaccgatatatccctgaagactattagcagagcttttatgaatggcagtgcactggaacatgtggtagagttcggcttagattacccagaaggcatggcagtggactggcttgggaagaacttatactgggcagacacaggaacaaatcgcattgaggtgtcaaagttggatggacagcaccgacaggttttggtatggaaagaccttgacagtccccgagctctggcattggatcctgctgaaggatttatgtattggactgagtggggagggaagcctaagattgacagagcggctatggatggaagtgaacgcactacattagttccaaatgtgggtcgagcaaatggccttaccatcgactatgctaagaggcggctttactggacagacctggacactaacttaatagaatcttcaaatatgctcggactcaaccgtgaagttatagcagatgacttgcctcatccttttggcttaactcagtaccaagattacatctactggacagactggagccgacgcagcattgaacgtgccaacaaaaccagtggccaaaaccgcaccatcatccagggccatttggattatgtgatggacatcctagtctttcactcttcccggcaggcagggtggaatgaatgtgcctccagcaacgggcactgctcccacctttgcttggctgtgcctgttggaggttttgtgtgtggatgccctgcccactactccctgaatgctgacaacaggacctgtagtgctcctaccaccttcctgctcttcagtcagaagagcgccatcaaccgcatggtgattgatgaacaacagagtcctgacatcatccttcctatccacagccttcggaacgtccgggccattgattatgaccctttggacaagcagctctactggattgactctcgacaaaacatgatacgaaaggcacaagaagatggtggccagggttttactgtagttgtaagctcggttccaaatcagaaccttgaaatacagccctatgatctcagcattgatatttatagccgttacatctattggacctgtgaagctaccaatgtcattgatgtgacgagattagatggtcgatcagttggagtggtcctaaaaggcgagcaagacagacctcgagccattgtggtaaaccctgagaaagggtatatgtattttaccaatcttcaggaaagatctcccaaaattgaacgggctgccttggatggaacagaacgggaggttctctttttcagtggcctgagtaagcccgtcgctttggctctcgacagcaagctgagcaggctcttctgggctgactccgacctccggcgaattgagagcagcgacctctcaggtgccaacaggatcgtgctagaggactctaatatattgcagcctgtgggtctgaccgtgtttgagaactggctctactggattgataagcagcaacagatgattgaaaaaattgacatgactggtcgcgaaggcagaaccaaggtccaggctcggattgctcagctcagtgacatccatgcagtgaaggagctgaaccttcaggaatacagacagcacccgtgtgcccaggataatggtggctgttcacatatttgccttgtaaaaggagatggtaccacaagatgctcctgtcccatgcatttagttctgcttcaggatgagctgtcctgtggagagcctccaacatgttctcctcagcagtttacgtgtttcactggggacattgactgcatccctgtggcctggcggtgtgatgggtttactgaatgtgaagaccacagtgatgagctcaactgtcctgtgtgctcggagtctcagttccagtgtgccagcgggcagtgcattgacggcgcccttcgatgcaatggtgacgcgaactgccaggataaatcggatgagaagaactgtgaagtgctttgtctaattgatcagttccgctgtgccaatggtcagtgcgttggaaaacacaagaaatgcgaccacaatgtggactgcagtgacagatctgatgaactggactgctattttgaacccaccaccatcctacaggccgtacagctaccggcactttgcaccgcccactacaccctgcagcactgatgtctgtgacagtgactatgctcctagccggagaatgacctcagtggcaacagccaagggctacaccagtgacttgaactatgactcagaacctgtgcccccaccacccacaccccgaagccagtacttgtcggcagaggagaactatgaaagctgccccccttctccatacactgagaggagttactcccatcacctctacccaccaccaccttccccctgcacggactcctcctgaggagggcccctcctcctctgactgcctccaccgggacatgtaaatacacatctggttgagatctggagggggggagggagctacagaaaagagtgaggcagacctgtacagttaacataaagtgggtggggtacctgagatatttgtacagaagaaaaggatatttatatattttcttaaaacagcagatttgctgcttgtgccataaaagtttgtataaaaaataaatttgtactaaaagttttatttttacaaactaaatacacagagcatgccttaagcccagtgaagaaactgagtacaaaggaagcgggacagtaaaggcatcactcaccaggaatgtctgggctttaatgataccaaaaatacaaaggagaaagtaatgccatctccaagtggccaacttggaaggagccaaattgccttcagggtagctaacatttgagggccccgaaagaaaaaagaacagtaccaactttggacaaagggctttgttttctccaacagttcacgaggaaagtagatatttgtgagatccattttgagtgttgctgtcgtaggagctgaagtgtctcttgcaggacgccgcagagctgctgagctctcacgcctgtcacagttcacgctaacaccagccgctttcacggtctgcctccccagcaccttggagacccgttttgctaagagccatgatttatttaaaaatgtgaggaattgagtaaatggaatttcctaaacgcgagattggact

SEQ ID NO: 390 - DNA, 5793 bp, Gallus gallus
atgatgcagatcccaccgtgtttcaaaagacaaaaagagaagtgcgataactttcaaaatcctggaactgagagatactgcgtactcttgcgctctgccccactcttgctgtatgcaaaccgccgtgacctgaggctggtggatgctgccaacgggaaggagaatgccacagtaatcgtgggtggcctcgaagatgcagcggcagtggactttgtgttcgtgaaggggttgatatactggagcgacgtgagtgaagaagccattaaacgaacagaattcaataaatccgggagtgtacaaaatgtggtcatttctggacttttgtcacctgatgggctggcatgtgactggctgggagagaaattatattggacagattctgaaacaaatcggattgaagtttcgaatttagatggatcattgagaaaagttttattttggcaagaattggatcaacccagagcaattgccttagatcctgcaagagggttcatgtattggacagactggggagaagtgccaaagatagaacgtgctgggatggatggttcaagccgctccattatagtcaacacggacatttattggccaaatggactgacattggattatgaggaacagaaactttattgggcagatgctaaactgaatttcatccacaaatcaaatctcgatggaagtcatcggcaagcagtggttaaaggctcccttccacatccttttgctttgacgctgtttggggacacattgtactggactgactggaacacgcattccatcttggcctgcagcaagtactctggggaggacctgcgtgaaatacattccaacatcttctcacccatggatatccatgctttcagccagaagaggcagccaaatgctacaaacccatgtggaattaataatggtggctgctcccacttatgtctgatgtctcctaccaagccctcttatcagtgtgcatgtcccactggtgtgaaacttcttgagaatggaaagacctgcaaagatggtgccactgaactgctgcttctagcccgccggacagatttgaggcgcatttccttagacactccagattttacagacatcgttttgcctctggaggacatccgtcatgccatcgccattgactttgatcctctggaaggatacatctattggactgacgatgaagttagagccatccgtcgctcattcgttgatgggtcaggtagtcagtttgttgtcacagcacagattgctcatcctgatggcattgctgtggactgggttgcccgaaatctgtattggactgacactggcacagaccgtatagaagttacaaggcttaatgggaccatgagaaaaatcttgatatcagaagacctggaagaacctagagctattgtgctggatcctatggtagggtacatgtattggactgactggggagaaattccaaagatcgagagggcagcattagatggctcggacaggattgtgctggtgaatacctcacttgggtggccaaatggactggcattggattatgcagaaagcaaaatatactggggagatgcaaaaactgacaaaatagaggtcatgaatgctgatggaactggaagaagagttcttgtagaggacaaacttcctcacatctttggattcacattattgggagactatgtttattggacagattggcaaagacgcagtattgaacgtgtgcataagcggacagcagaaagagaaatcatcatagatcaactgcctgatctaatgggcctcaaagctacaaatgtacaccagataattggtacaaacccttgtgcagaggataatggtggttgcagccatctgtgtctgtacagaccgcaaggccttcgctgcgcttgtcccataggcttggagctcatcaatgacatgaagacctgcattgtcccagaagctttccttctgttttctcggagagcagatattagacgaatctctttagaaacaaacaacaacaacgttgctattccacttactggagttaaggaagcttccgctctggatttttgatgtgacagacaatcgaattactggactgatatttcattgaagaccatcagcagagcttttatgaatggcagcgcactggaacacgttgtagagtttggcttggattaccctgaaggcatggccgtagattggctggggaagaacttgtattgggctgatactggaacaaatcgcatcgaagtgtcaaagctggacggccagcatcgccaagtgttggtgtggaaagatcttgacagtccccgggcactggcactggatcctgcagaggggtttatgtactggactgaatggggtggtaaaccgaagattgatagagcttctatggatggcagtgagcggactactctggtacccaatgtgggacgtgccaacggcctaacaattgattatgctaaaagacgactgtactggactgaccttgataccaacctgatagaatcctccaacatgctaggccttgaccgtgagattatagctgatgacttgcctcacccattcggcttgactcagtatcaggattacatttactggacagactggagccggcgcagcattgaacgagccaacaagacgagtggccagaacagaactatcatccaagggcatttggattatgttatggacatcctggtcttccattcctccaggcaggcaggctggaacgagtgtgcctctagcaatggccattgctctcatctctgcttagcagtccctgttggtggttttgtctgcgggtgccctgctcactactctctgaactctgacaacaggacttgcagtgctcctacaacctttcttttgttcagtcagaagaatgcaattaaccgcatggtgatagatgagcagcagagtccagatatcatacttcctatccatagtctccgaaatgttcgagccatcgattatgaccccctggataagcagttgtattggattgattcgcggcagaacatcattcggaaggcacaggaggatggcagccagagcctcactgttgtgataagtccagttcccaatcagaacctagacatgcagccttatgacctgagcattgacatctacagccgctatatctactggacctgtgaagctactaatgtcatcaatgtgacacgcctggatgggagacccatgggagtggtgctcaaaggagatcaagatagacccagggccattgtggtgaatccagagaaaggatacatgtatttcaccaacctgcaggaaaggtctcctaaaattgagagagcagcattggatggcactgaacgagaggtcctttttttcagtggtttgagcaagcctatagccttagctattgacagccagctaggcaagttgttctgggctgattcagacctgcggagaattgaaagcagtgacctttcaggtgctaaccgagttgttttggaagattccaatattttgcaacctgtgggactaactgtatttgagaactggctgtattggattgacagacaacagcagatgattgaaaagattgatatgactggtcgagaaggacgtacaaaggtccaagctcgtattgcacaactcagcgacattcatgctgtgaaagagcttaacgttcaggaatacagacaacacccttgttctcaagataatggtggttgctcacacatttgcattgtgaagggcgatggcaccacacgctgctcttgtccagtccaccttgttttgctgcaggatgagctgtcctgtggagaacctccaacatgctcccctcagcaattcacctgtttcacaggtgagattgactgcatcccggtggcctggcgctgtgatggtttcactgaatgtgaagaccacagtgatgaaaaaaactgcccagtgtgctctgacacccagtttcagtgtgaaagtggacagtgcatagacagtgccctccggtgcaatggagaagcaaattgccaggacaactcagatgagaagaactgtgaagtgctctgtttaaccagtcagttccggtgtgccagcgggcagtgcattggtaaaagcaagaaatgtgatcacaacctggactgtagtgacagctcagatgagcaaggatgctacacgacagaggagcctgcgccacagcccaacaacacaataggctccatcattggtgtaatcctcacactttttgtggtcggagcaatgtatttcatctgccagagggtgctgtgcccacgcatgaaaggagatggagagacaatgaccaatgattatgtggtacatggaccagcctctgtacctcttggttatgtgcctcacccaagctctttgtcagggtctcttcctgggatgtctcgaggaaaatctgtgatcagctccctcagcattatgggagggagcagtgggccaccctatgacagggcccatgtcactggagcatcttccagtagttcttcaagtacgaaaggcacttactttcctccaattttgaacccaccaccatcaccagctactgaacgttcacattatacaatggaattcggttattcttcaaacagcccatccactcatagatcttacagctacaggccttacagctaccgccactttgcacctcccacaacaccctgtagcacggatgtctgtgacagtgactatgccccaagccgaagggtcacggccacaatggccaagggctacaccagtgacttgaactatgactcagagcccgtcccaccgcctccgaccccacgcagccagtacctgtcagcggaggagaactacgagagctgccccccttcaccatacacagagcggagctactctcaccacctctacccaccaccaccgtccccctgcacagactcctcatgaggggcagccccccctccattatctgcctctgccgtggaagtgtaaatacaaagtgttctaccagggaggggggagttcttggcaaggggtgaggtagaacagtgtacagttaaatgttattcagttgggtggaggaatactggacatatttgtacagaagaaaaaaaggatatttatatatttttttaaacagcaactgctgcttgtgccataagaaaaatttttgtataaaagaacccagacatttgtacaaaaagtttttatttttgcaaattgaacacaaaaacatgccttaatccagtgaagtgactgagcacatagcgaaatggaaggaccaggtatgtgtcacttgtccagagaggtgagatgtggggtttgtcaataccaaaaatgtttaaagtaattaatgataaagaagtccgtctcagagcagcataccatagatacttggaagtagccaaataacctctatattagctacagagggccagcaactaagttaaacttgaaaatgcagtcaggacagataataaccttatacaaagggctttgttttctacaggaatacagcaatcgtagcagtgaatccagaagaataatcacacacttttttaaaagagtacccctctgtttttcttcaatacatttaccaaacttgagcattttgagctctctccttgcttctggaatttcaatgatttttgttgggtatattttcaagtctggtttgttaatactctttagcctttctcatctcttctacatcctttcttccctcccatgtcactttaaatcacattatccttatttaaagtattgctagaactgggctgctgtaaattcttttttttttcccctcctaattagccttgtcgtgcaatgagtgtcatgctatacattacttcattaattttg

SEQ ID NO: 391 - DNA, 968 bp, Mus musculus
ccacgcgtccggaaagaattagcggctcctcagcccagcaaatcctccactcatcatgcttcctcctgccattcatctctctctcattcccctgctctgcatcctgatgagaaactgtttggcttttaaaaatgatgccacagaaatcctttattcacatgtggttaaacctgtcccggcacaccccagcagcaacagcaccctgaatcaagccaggaatggaggcaggcatttcagtagcactggactggatcgaaacagtcgagttcaagtgggctgcagggaactgcggtccaccaaatacatttcggacggccagtgcaccagcatcagccctctgaaggagctggtgtgcgcgggcgagtgcttgcccctgccggtgcttcccaactggatcggaggaggctatggaacaaagtactggagccggaggagctctcaggagtggcggtgtgtcaacgacaagacgcgcacccagaggatccagctgcagtgtcaggacggcagcacgcgcacctacaaaatcaccgtggtcacggcgtgcaagtgcaagaggtacacccgtcagcacaacgagtccagccacaactttgaaagcgtgtcgcccgccaagcccgcccagcaccacagagagcggaagagagccagcaaatccagcaagcacagtctgagctagacctggactgactagaaagcatctgctacccagatttgattgcttggaagactctctctcgagcctgccattgctctttcctcacttgaaagtatatgctttctgctttgatcaagcccagcaggctgtccttctctgggactagcttttcctttgcaagtgtctcaagatgtaatgagtagtttgcagtgaaagccaggcatcctgtagtttccatcccctcccccatcccagtcatttctttaaaagcacctgatgctgcattctgttacagtttaaaaaaaaaaaaaaa

SEQ ID NO: 392 - DNA, 1723 bp, Homo sapiens
acagttacagtgcagaagcagaggcaaaagaattaaccagctcttcagtcaagcaaatcctctactcaccatgcttcctcctgccattcatttctatctccttccccttgcatgcatcctaatgaaaagctgtttggcttttaaaaatgatgccacagaaatcctttattcacatgtggttaaacctgttccagcacaccccagcagcaacagcacgttgaatcaagccagaaatggaggcaggcatttcagtaacactggactggatcggaacactcgggttcaagtgggttgccgggaactgcgttccaccaaatacatctctgatggccagtgcaccagcatcagccctctgaaggagctggtgtgtgctggcgagtgcttgcccctgccagtgctccctaactggattggaggaggctatggaacaaagtactggagcaggaggagctcccaggagtggcggtgtgtcaatgacaaaacccgtacccagagaatccagctgcagtgccaagatggcagcacacgcacctacaaaatcacagtagtcactgcctgcaagtgcaagaggtacacccggcagcacaacgagtccagtcacaactttgagagcatgtcacctgccaagccagtccagcatcacagagagcggaaaagagccagcaaatccagcaagcacagcatgagttagaactcagactcccataactagacttactagtaaccatctgctttacagatttgattgcttggaagactcaagcctgccactgctgttttctcacttgaaagtatatgctttctgctttgatcaaacccagcaagctgtcttaagtatcaggaccttctttgggaatagtttttccttttcaagtttttcaagatgtaggtatatccatgaatgcaatttgcatttaaattccacgtatcctgtagttttaattcctcattgttcttaaaagactgttgatactataaacatcagtgaatcattatattttaaaacagaaaagggcttctcagataccctccatctactggcccatcccctctcctaaacaaaactccttcaaaacaggttaaaaaaaatatgttgtcatgaatcttcacagtaacatttcagaaaggtgcttttttggtactcttcatgggaacagtttagcagccatgagtgatcttcctttgaaagagaatgaaagaccctgtgacatttcacttcaaaaataagccctgtagctctttacggtcgcatagtataaaattataccctgcatgctgaccctcgcttggaatggaatgccagaaatgcatggcagcagctaataagtaaagctgattaactatttatttgtcaatgttattatttaatgagctttcacatgtgatttgtttcaaaactttaattttttaatgttttgaaactttttcatggacctaaatattttcctatatgatttgtggttgattagaaatatgaaatacatgttgtagatatgtaaaatgaatattttagtctccttattacatatatgttcatggtgaactttatcaatagtatggatctttttaaatcaataagatgctttgtaaagttgaaataagtaatactttcttgtttaatctgtgcaatcagaaggtgtcttgaccttcaattcaattggtttcttttaacaaaaataaacactgctaaaagcaaaaaaaaaaaaaaaaaaaaaaaaaaaaa

SEQ ID NO: 393 - DNA, 1698 bp, Rattus norvegicus
gcagggggaaagaattcaccgcctcagccgagtaaatcctccactcatcatgcttcctcctgccattcatctctctctcattcccctgctctgcatcctgatgaaaaactgtttggcttttaaaaatgatgccacagaaatcctttattcacatgtggttaaacctgtttcagcacaccccagcagcaacagcaccttgaatcaagccaggaatggaggcaggcacttcagtagcacgggactggatcgaaatagtcgagttcaagtgggctgcagggaactgcggtccaccaaatacatctcggatggccagtgcaccagcatcagccctctgaaggagctggtgtgcgcgggtgagtgcttgcccttgccagtgcttcccaactggatcggaggaggctacggaacaaagtactggagccggaggagctcccaggagtggcggtgtgtcaacgacaagacgcgcacccagagaatccagctgcagtgtcaggacggcagcacacgcacctacaaaatcaccgtggtcacagcgtgcaagtgcaagaggtacacccggcagcacaacgagtccagccacaactttgaaagcgtgtctcccgccaagcccgcccagcaccacagagagcggaagagagccagcaaatccagcaagcacagtctgagctagagctggactgactagaaagcatctgctccccagatttgattgcgtggaagactcgctcgagcctgccattgctcttttctcacttgaaagtatatgctttctgctttgatcaggcccagcaggctgtccttctctgggaccttctctgggactagcttttcctttgcaagtttctcaagatgtaatgagtagtttgcagtgaaagccaggcatcccatagtttcaccccctcccccagtcatttctttcaaaagcacctgatgctgcatcctgttacagtttaaaccgcgctccattccctagcccaccctttacccttcccaaactaaatccctccgaaccggagaagttcaatcaaaaaaaaaatgtatcttcacagaacatttcacaaaggggcttttccagtagtttttatgggaatagtttgacagccaggggttagcttcctttgaaagagaggcaaaccttgtgacatttcacttcaaaaataagcccggtggcattttccagtctcagtgtgaaattatccccctcgtgttgacctctcttggagtggaatgccagcaatgcaaggcagcagctagtaggtaaagccggttatttatttgtcgatgttgttatttaatgagctcgcgcatgtgattttttttccaaatgttaattttttttatattttgaagctttttcatgtacctaaatatttcccaatacgatttgtggttggtctagaaatatgaaaatacatgttgtagatatgtaaaaataaatattttagactccttatatattacacggttcctcatacactttgtcattagcgtggttccctgtaaagctgaagttatgtaatactttattatttagtccatgaaatcagaaggcagcttgaccttcggtaccatcggtttcttttaataaacataaatatccactaaatgctgttgtctttgcctctgacatgcatgcttgcagatactgtaaaataaagttataagtgtgaaaaaaaaaaaaaaaaaaaaaaaaaaaaa

SEQ ID NO: 394 - DNA, 1433 bp, Gallus gallus
cacaactttacacctgaatgaatgccaagcctctccgaatataaaaaggaaaccttgaagagcaatttcagagttagcgagaacgagcagaagaaaaacatttcatccggctccgtcagacagagaccaaaccaatcatgcttctctccgccattcacttctacggcttactcctagcttgcaccttcacgagaagctactcggctttcaagaacgatgccactgagatactttattcccacgtcgttaaacctgcccctgcgagcccgagcagcaacagcacgttgaaccaagccaggaacggagggaggcactacgccggcacgggctccgaccgtaacaatcgcgttcaagttggctgccgggaactgcgatctaccaagtacatctcagacggccagtgcaccagcatcaatcccctgaaggagctggtgtgtgctggcgaatgcctccccttgccgctcctgcccaactggattggaggaggttatggaaccaagtactggagcagacggagctcgcaagagtggagatgtgtcaatgacaaaactcgcacccagaggatccagctgcagtgccaggatggaagtataagaacctacaaaataactgtggtcacggcctgcaagtgcaagcgatacaccaggcagcacaacgagtccagccacaactttgagggaacctctcaagcaaagcctgtccagcatcacaaagagagaaaaagagccagtaaatccagcaaacatagtacaagttagaagtcagacgtcctctcctctcctgctcctcctgccaaaactgaactcacctgtaaccatttgctttacagttctgactgcttaaagacaagtctgccattgctttgttctcagatgatactgcacgcttattgttttgaccaaaatcaaaaggggcattctcagcatatggacctttttgggtgactgtccaaaagctcaagatgggcagtaacacaagccttctaaaccctcacttcaaagttttacattccctccagccacggaggcaaagaaaagttgccatgaatattcacggaagtctgttttgtaaaatggtgctttgttgtgtgtcttcacgagaacagtttattatcactcctatcgccttctctatcgatgttttatgaacttctgacaacagcttacaaatgcaacaccaactatctgctttcagattcattctaagaaaagaatgccttgcatgctgatctttcccctcagtataccaaaaatgaaaatatgcatggcagcaatccaaaagtaacgcggaacaaactatttatttgttgttgtaattatttaatgagcttttgtattatttttgtttaaaagtgtgcaatcaaaagacagatatgacttcctttccaattcattggtttctttttttttttgtacaaaaataaacactgctaa

(19) United States
(12) Patent Application Publication
(10) Pub. No.: US 2019/0194314 A1
(43) Pub. Date: Jun. 27, 2019

(54) ANTIBODIES FOR MODULATING BINDING BETWEEN LRP AND WISE

(71) Applicant: STOWERS INSTITUTE FOR MEDICAL RESEARCH, Kansas City, MO (US)

(72) Inventors: Robb Krumlauf, Mission Hills, KS (US); Debra Ellies, Kansas City, MO (US)

(21) Appl. No.: 14/653,759

(22) PCT Filed: Dec. 20, 2013
(86) PCT No.: PCT/US13/77299

§ 371 Date: Jun. 18, 2015

Related U.S. Application Data
(60) Provisional application No. 61/745,007, filed on Dec. 21, 2012.

(51) Int. Cl. C07K 16/28; C07K 14/705

(52) U.S. Cl. CPC C07K 16/28; C07K 2317/30; C07K 2317/76; C07K 14/705

(57) ABSTRACT

The present invention provides, inter alia, antibodies that modulate binding between Lrp5 and WISE or Lrp6 and WISE, but do not modulate binding between Lrp4 and WISE. Also provided are pharmaceutical compositions and kits containing such antibodies. Further provided are methods for preventing WISE binding to Lrp5 or Lrp6, but not WISE binding to Lrp4.

### CROSS-REFERENCE TO RELATED APPLICATIONS

The present invention claims benefit to U.S. provisional application Ser. No. 61/745,007 filed Dec. 21, 2012, the entire contents of which are incorporated by reference.

### FIELD OF INVENTION

The present invention provides, inter alia, antibodies that modulate binding between Lrp5 and WISE or Lrp6 and WISE, but do not modulate binding between Lrp4 and WISE.

### INCORPORATION BY REFERENCE OF SEQUENCE LISTING

This application contains references to amino acids and/or nucleic acid sequences that have been filed concurrently herewith as sequence listing text file “0339588pct.txt”, file size of 389 KB, created on Dec. 20, 2013. The aforementioned sequence listing is hereby incorporated by reference in its entirety pursuant to 37 C.F.R. § 1.52(e)(5).

### BACKGROUND OF THE INVENTION

Wnt signaling plays an important role in a variety of processes, including development and maintenance of various organs and tissues, such as the bones. Mutations in Wnt genes or Wnt pathway components lead to specific developmental defects, including defects in the formation of mammary placodes, while various human diseases, including cancer and alterations in bone mass, are caused by abnormal Wnt signaling (For a review, see, e.g., Krishnan et al., 2006).

In the Wnt/β-catenin signaling pathway, interaction of Wnt ligands with Frizzled (Fz) receptors and Wnt co-receptors, Lrp5 and Lrp6, initiates a series of intracellular events leading to stabilization and nuclear accumulation of β-catenin. Subsequently, β-catenin forms complexes with TCF/LEF transcription factors and activates expression of target genes (MacDonald et al., 2009). Ectopic expression of the Wnt inhibitor Dickkopf 1 (Dkk1) blocks placode formation (Chu et al., 2004) and lack of Lef1, Lrp5 or Lrp6 disrupts normal placode development (van Genderen et al., 1994; Boras-Granic et al., 2006; Lindvall et al., 2006; Lindvall et al., 2009). It has been shown that Wnt/β-catenin signaling is initially activated in a broad domain along the mammary line, coincident with the expression pattern of a number of Wnt genes, but rapidly becomes restricted to mammary placodes (Chu et al., 2004; Veltmaat et al., 2004). This suggests that spatiotemporal control of the signaling activity is tightly coupled to placode formation. However, little is known about how precise control of Wnt signaling is achieved during embryonic mammary development.

Modulation of Wnt/β-catenin signaling in the extracellular space is often mediated by secreted Wnt antagonists, which interact with Wnt proteins, Fz receptors or Lrp5/6 co-receptors (MacDonald et al., 2009). For example, Dkk1, Sost and Wise (Sostdc1-Mouse Genome Informatics) can bind to the extracellular domain of Lrp5/6 and inhibit Wnt signaling presumably by disrupting the formation or activity of Wnt-induced Fz-Lrp5/6 complexes (Semenov et al., 2001; Itasaki et al., 2003; Li et al., 2005; Semenov et al., 2005). Another layer of complexity was added by recent findings on a low-density lipoprotein (LDL) receptor-related protein, Lrp4. The extracellular domain of Lrp4 resembles that of Lrp5/6, but its intracellular domain is distinct from that of Lrp5/6 suggesting that it may have different inputs on Wnt signaling (Herz and Bock, 2002; Weatherbee et al., 2006). In humans, LRP4 mutations cause limb, kidney and tooth malformations in Cenani-Lenz syndrome and are associated with bone overgrowth in two isolated cases of sclerosteosis (Li et al., 2010; Leupin et al., 2011). The role for Lrp4 appears to be conserved in mammals, because mice deficient for Lrp4 also display defects in limbs, kidney and teeth (Johnson et al., 2005; Weatherbee et al., 2006; Ohazama et al., 2008).

In Lrp4 mutant mice, limb and tooth defects were associated with abnormal Wnt signaling activity. Furthermore, Lrp4 can antagonize activation of Wnt signaling when over-expressed in cultured cells and this inhibitory activity is lost in mutant proteins (Johnson et al., 2005; Li et al., 2010). However, studies in bone and kidney development revealed no apparent elevation of Wnt signaling in Lrp4 mutants (Choi et al., 2009; Karner et al., 2010). In addition, Lrp4 is implicated in regulation of Bmp signaling in some contexts and functions as a co-receptor for Agrin in the neuromuscular junction (Kim et al., 2008; Ohazama et al., 2008; Zhang et al., 2008). Therefore, whether Lrp4 directly inhibits the Wnt pathway or it controls another pathway to indirectly affect Wnt signaling in vivo has been unclear.

Similar to Lrp5/6, Lrp4 can bind in vitro to Dkk1, Sost and Wise suggesting that roles for Lrp4 in Wnt signaling may be modulated by binding of these antagonists (Ohazama et al., 2008; Choi et al., 2009; Karner et al., 2010). This is consistent with the observation that Lrp4 facilitates the Wnt inhibitory function of Sost in in vitro bone mineralization (Leupin et al., 2011). In addition to this potential cell-autonomous role as a membrane receptor, Lrp4 is also postulated to modulate Wnt signaling by releasing its extracellular domain, and hence sequestering Wnt antagonists (Choi et al., 2009; Dietrich et al., 2010). It remains to be determined whether interaction between Lrp4 and the Wnt antagonists plays a significant role in vivo.

Similar to other LDL receptor-related proteins, Lrp4 is implicated in regulating different signaling pathways (May et al., 2007; Willnow et al., 2007). With its multiple ligand binding motifs in the extracellular domain, Lrp4 has the ability to bind in vitro to secreted Wnt and Bmp antagonists (Ohazama et al., 2008; Choi et al., 2009). Interestingly, in both humans and mice Lrp4 mutations phenocopy defects caused by deficiency of individual Wnt antagonists in a tissue-specific manner. For example, limb defects of Lrp4 mutants are similar to those of Dkk1 mutant mice (MacDonald et al., 2004), and bone overgrowth of human patients with LRP4 mutations is reminiscent of bone defects caused by SOST and Dkk1 mutations (Balemans et al., 2001; Morvan et al., 2006). Lastly, Lrp4 and Wise mutant mice share defects in tooth, mammary glands and other skin appendages (Ohazama et al., 2008). These observations imply that interplay between Lrp4 and the Wnt antagonists may play an important role in modulating Wnt/β-catenin signaling in many developmental and physiological contexts.

Wise is known as a context-dependent modulator of Wnt signaling and an inhibitor of Bmp signaling (Itasaki et al., 2003; Laurikkala et al., 2003; Lintern et al., 2009). The strong genetic interaction of Wise with Lrp5 and Lrp6 suggested that Wise controls tooth number and patterning by inhibiting Wnt signaling (Ahn et al., 2010).

Genetic studies in mouse have provided insights on signaling pathways required for embryonic mammary development (Robinson, 2007). Skin appendages such as teeth, hair and mammary glands develop from the surface ectoderm and underlying mesenchyme during embryogenesis. Despite the differences in the final structures, these skin appendages arise through similar morphological processes and tissue interactions in the early stages of their development (Mikkola and Millar, 2006). The future site of appendage development is initially marked by a thickening of the epithelium, which gives rise to a more localized placode. Subsequently, invagination of the placodal epithelium and condensation of the underlying mesenchymal cells leads to bud formation. Interactions within and between epithelial and mesenchymal tissues are essential for the proper growth and patterning of placode development. Genetic disruptions of genes encoding components of signaling pathways (Wnt, FGF, BMP, Eda, etc.) often cause developmental defects in multiple skin appendages suggesting that patterning processes are shared among these appendages at the molecular level (Pispa and Thesleff, 2003; Mikkola and Millar, 2006).

While many aspects of early patterning are similar, the spatial and temporal dynamics of placode development appear to be unique among the appendages. For example, hair placode formation begins with broad, regularly spaced epithelial thickenings, which are gradually refined to smaller circular placodes (Schmidt-Ullrich and Paus, 2005). In contrast, mammary placodes develop along the mammary lines, two lines of transient epithelial thickening, which appear between the fore and hind limb buds. Within a day, five pairs of mammary placodes form in a defined order as the mammary lines resolve (Robinson, 2007; Cowin and Wysolmerski, 2010) (FIG. 1C). The molecular and cellular basis of this transition is still unclear. However, earlier morphological studies in rabbits and recent cell-tracing experiments in mice suggested that the formation and growth of mammary placodes involve migration and reassembly of the mammary epithelial cells (Propper, 1978; Lee et al., 2011). This dynamic mode of placode formation suggests that mammary glands may have adopted a distinct molecular mechanism for placode induction.

Accordingly, there is a need to clarify the relationship between Lrp4 and Wise, provide insights into interplay between Lrp4 and Wnt antagonists in Wnt inhibition, and to provide improved regulation of the interaction between Lrps and Wnt antagonists. The present invention is directed to meeting these and other needs.

### SUMMARY OF THE INVENTION

One embodiment of the present invention is an antibody that modulates binding between Lrp5 and WISE or Lrp6 and WISE, but does not modulate binding between Lrp4 and WISE.

Another embodiment of the present invention is a pharmaceutical composition. This pharmaceutical composition comprises one or more antibodies of the present invention and at least one pharmaceutically acceptable excipient or diluent.

A further embodiment of the present invention is a method for preventing WISE binding to Lrp5 or Lrp6, but not WISE binding to Lrp4, comprising contacting Lrp5 or Lrp6 with an agent that binds to Lrp5 or Lrp6 but not Lrp4.

An additional embodiment of the present invention is a method for preventing human WISE from binding to human Lrp5 or human Lrp6 comprising contacting human WISE with a monoclonal antibody, which antibody specifically binds to human Lrp5 or human Lrp6 but not to human Lrp4.

A further embodiment of the present invention is a kit. This kit comprises one or more antibodies of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the abnormal mammary development in Lrp4 mutant mice. FIG. 1A shows five pairs of nipples (#1-5) in a pregnant control female. FIG. 1B shows that Lrp4 mutant female displays ectopic nipples (arrowheads) and fusion of nipples #2 and 3. FIG. 1C is a cartoon showing appearance of the five mammary placodes on each side of the embryos (top) and distribution of mammary epithelial cells around placodes #2 and 3 (bottom). FIG. 1D shows that TopGal is expressed in mammary epithelial cells, which are gradually restricted to placodes in control. FIG. 1E shows that in Lrp4 mutants, delayed placode formation (E12.0) is followed by ectopic buds (arrows) and fusion of placodes #2 and 3. Higher magnification images (E12.0) and histological sections (E12.5 and E14.5) of the placode #2 and 3 area are shown in bottom panels in FIGS. 1D and 1E.

FIG. 2 shows the genetic interaction of Lrp4 with Lrp5 and Lrp6. FIGS. 2A, 2B, and 2B′ show that reduced dosages of Lrp5 and Lrp6 rescue the limb (FIG. 2A) and mammary (FIGS. 2B and 2B′) defects of Lrp4 mutants. FIGS. 2C and 2C′ show that Lrp4 and Lrp6 compensate for loss of each other in limbs. Note that hindlimb defects of Lrp6^{-/-} mice were rescued by inactivation of Lrp4 (FIG. 2C′), but other defects such as loss of tail remain the same (arrows). FIG. 2D is a chart showing the separation of mammary buds #2 and 3 by reduced dosages of Lrp5 and Lrp6 in Lrp4 mutants. In this Figure, TopGal expression at E13.5 is shown. A proximal (left, dorsal to the right) and a dorsal (right) view of a forelimb bud are shown for each genotype with anterior to the top in FIG. 2A. A low level of broad β-galactosidase activity is detectable from LacZ inserted into the Lrp6 mutant allele.

FIG. 3 shows that Lrp4 inhibits Wnt/β-catenin signaling to facilitate placode initiation and control the number of mammary epithelial cells. FIGS. 3A-3C and 3A′-3C′ show that reduced dosage of Lrp5 and Lrp6 restores normal timing of placode initiation and reduces ectopic TopGal-expressing cells in Lrp4 mutants. FIGS. 3D-3F and 3F′ show the detection of Cre activity from K14cre transgene using the R26-floxstop-LacZ line. FIGS. 3G-3J and 3G′-3J′ show that epithelial β-catenin is required for growth of mammary buds, as shown in FIGS. 3H and 3H′. Inactivation of β-catenin in Lrp4 mutants results in separated, but smaller buds, as shown in FIGS. 3J and 3J′.

FIG. 4 shows that Lrp4 is required for development of other skin appendages. FIG. 4A shows delayed formation of the primary hair follicles in Lrp4 mutants as evidenced by the lack of Wnt10b expression. FIGS. 4B-4E, 4D′, and 4E′ show that the expression of the Lrp4-LacZ BAC reporter line is observed in early hair placodes and mammary buds (#1-5) at E13.5. The mammary buds 1-5 at E13.5 are as indicated in FIG. 4B. More focalized reporter expression is observed in mature hair placodes of back skin at E14.5, as shown in FIGS. 4D and 4D′. In Lrp4 mutants, Lrp4-LacZ expression is spread along the mammary line (arrow) with no sign of hair placodes at E13.5, as shown in FIG. 4C. The mutants show a delay in hair placode development, as shown in FIGS. 4E and 4E′). FIGS. 4F and 4G show supernumerary mystacial (arrow) and supra-orbital (arrowhead) vibrissal follicles in Lrp4 mutants. FIGS. 4H, 4I, 4H′, and 4I′ show the abnormal patterning of interramal vibrissal follicles in Lrp4 mutants. Frontal sections, shown in FIGS. 4H′-4I′, were obtained along the dashed line shown in FIGS. 4H-4I. FIGS. 4J-4M show that Lrp4 mutants display supernumerary vibrissal follicles in the submental (rectangle), postoral (circle) and interramal (oval) regions.

FIG. 5 shows that Wise controls patterning of mammary placodes via the Wnt/β-catenin pathway. FIGS. 5A and 5A′ show whole-mount in situ hybridization (FIG. 5A) and cross-section across the mammary placodes #2 and 3 (FIG. 5A′). FIG. 5B shows that Wise-null females display abnormal spacing between nipples and supernumerary nipples (arrowheads). FIGS. 5C and 5D show that in mutants, abnormal size and morphology of the placodes is apparent by E12.5. The distance between placodes #2 and 3 is reduced, often leading to fusion at later stages. TopGal-expressing cells are ectopically observed in the interplacodal regions. FIGS. 5E and 5F show that the loss of Lrp5 or epithelial inactivation of β-catenin restores normal spacing between mammary buds #2 and 3 in Wise-null mice.

FIG. 6 shows that Wise over-expression disrupts mammary development. FIG. 6A shows a schematic diagram of K14-tTA and tetO-Wise constructs. FIGS. 6B-6C and 6B′-6G′ show that, Wise over-expression disrupts limb development (arrow) and results in smaller mammary placodes. FIGS. 6D-6G and 6D′-6G′ show that Wise-null mammary defects are rescued by a moderate level of Wise expression in the ectoderm. FIG. 6H shows a schematic diagram of the TCF-tTA construct. FIGS. 6I-6K and 6I′-6K′ show that TCF-tTA;tetO-Wise mice display limb and mammary defects. TopGal is shown in FIGS. 6B-6G, 6B′-6G′, 6I-6J, and 6I′-6J′, and eGFP expression is shown in FIGS. 6K and 6K′.

FIG. 7 shows the genetic interaction between Lrp4 and Wise. FIGS. 7A-7C show TopGal expression in various Lrp4; Wise double mutant mice. Transheterozygotes display normal mammary patterning (FIG. 7A), and inactivation of Wise does not exacerbate Lrp4 mutant defects such as fusion of bud #2 and 3 and ectopic buds (arrows in FIG. 7B-7C). FIGS. 7D-7I and 7G′-7I′ show that Wise over-expression, as evidenced by eGFP expression (FIGS. 7G′-7I′), fails to rescue the mammary (FIGS. 7D-7E and 7G-7H) and forelimb (FIGS. 7F and 7I) defects of Lrp4 mutants, as demonstrated by the lack of significant change in TopGal expression. FIGS. 7J-7M and 7J′-7M′ show that Wise over-expression causes reduction in the number of vibrissal follicles (FIGS. 7J and 7L) and taste papilla (FIGS. 7J′ and 7L′), but has no significant effect in Lrp4 mutants (FIGS. 7K, 7M, 7K′, and 7M′). All at E14.5 except FIGS. 7F, 7I and 7I′, which are at E13.5.

FIG. 8 shows a model of the function of Lrp4 and Wise in mammary development. FIG. 8A shows that Wnt/β-catenin signaling modulates multiple steps of placode formation. Initially, Lrp4 facilitates placode initiation, and later Lrp4 and Wise together limit the number of mammary precursor cells by inhibiting Wnt/β-catenin signaling. FIG. 8B shows that the activation of Wnt/β-catenin signaling requires formation of the Wnt-Frizzled receptor-Lrp5/6 complex, which eventually leads to regulation of target genes by β-catenin and TCF/LEF transcription factors. Early (the left panel of FIG. 8B), Lrp4 functions in a Wise-independent manner, but later (the right panel of FIG. 8B), Lrp4 and Wise act together to inhibit Wnt signaling.

FIG. 9 shows the similarity between mammary defects in different Lrp4 mutant mice. FIGS. 9A-9D show that allelic combinations of Lrp4 mutations result in abnormal patterning of mammary placodes as shown by TopGal expression. Ectopic TopGal-expressing cells are present in the interplacodal regions of mutant mice (arrows).

FIG. 10 shows that β-catenin is required for growth of mammary buds. FIGS. 10A-D, 10C′, and 10D′ show the ectodermal inactivation of the β-catenin gene leads to hypoplastic mammary buds.

FIG. 11 shows that Wise is required for skin appendage development. FIGS. 11A and 11B show complementary expression patterns of TopGal and Wise-LacZ in the mystacial (red circles), supra- and sub-orbital (green circles) vibrissal follicles, mammary buds (pink circles) and hair follicles (yellow circles). FIGS. 11C and 11D show that BrdU staining is reduced in the epithelium between mammary buds #2 and 3 (arrow) in Wise-null mice. FIGS. 11E and 11F show that TopGal expression reveals supernumerary mystacial (arrow) and supra-orbital (arrowhead) vibrissal follicles in Wise-null mutant mice.

FIG. 12 shows that over-expression of Wise disrupts development of limbs and skin appendages. FIG. 12A shows a schematic diagram of the K14-Wise construct. FIGS. 12B-12E show that K14-Wise mice display hair loss and limb abnormalities. FIGS. 12F-12G show that histological sections reveal disruption in formation of primary hair follicles in K14-Wise mice. FIGS. 12H-12I and 12H′-12I′ show the abnormal development of mammary placodes and limb buds in K14-Wise mice.

FIG. 13 shows a sequence comparison of human Lrp4, 5, and 6. The highlighted areas show some of the exemplary sequences to which an antibody that modulates binding between Lrp5 and WISE or Lrp6 and WISE, but does not modulate binding between Lrp4 and WISE would specifically recognize.

FIGS. 14A-K show increased number of mammary epithelial cells in Lrp4 mutants. FIGS. 14A and 14B are confocal images of the placode 2/3 region from TCF/LEF:H2B-GFP embryos. FIG. 14C shows the relative number of GFP-positive cells in FIGS. 14A and 14B. Data are mean±s.d. FIGS. 14D-14G show that BrdU staining is reduced in the interplacodal region (arrow) in Lrp4 mutants. FIGS. 14H and 14I show caspase 3 staining. FIGS. 14J and 14K show E-cadherin staining. Note that placode 2 is out of the focal plane in FIGS. 14F and 14J.

FIG. 15 shows reduced number of proliferating cells in the interplacodal epithelium of Lrp4 mutant mice. Relative number of BrdU-positive epithelial cells between mammary placodes 2 and 3 at E12.5 on stained sections were determined. Average number of labeled cells in control mice is 27.8. Data are mean±s.d.

FIGS. 16A-16C show a gene expression analysis of Lrp4 mutant mammary placodes. Real-time PCR was performed with TaqMan assays (Life Technologies) using cDNA from mammary placodes 2/3 and surrounding epithelial and mesenchymal tissues dissected from E12.5 embryos. Twelve to 14 dissected areas (the areas marked with rectangles in FIG. 16B) from control and mutant embryos were pooled for RNA extraction. The error bar was calculated from four replicates for each probe using DataAssist (Life Technologies). FIGS. 16B and 16C are in situ hybridizations showing increases in Lef1 (B) and Msx1 expression.

### DETAILED DESCRIPTION OF THE INVENTION

One embodiment of the present invention is an antibody that modulates binding between Lrp5 and WISE or Lrp6 and WISE, but does not modulate binding between Lrp4 and WISE.

As used herein, an “antibody” encompasses naturally occurring immunoglobulins as well as non-naturally occurring immunoglobulins, including, for example, single chain antibodies, chimeric antibodies (e.g., humanized murine antibodies) and heteroconjugate antibodies (e.g., bispecific antibodies), as well as antigen-binding fragments thereof, (e.g., Fab′, F(ab′)₂, Fab, Fv, and rlgG). See also, e.g., Pierce Catalog and Handbook, 1994-1995 (Pierce Chemical Co., Rockford, Ill.); Kuby, J., Immunology, 3rd Ed., W. H. Freeman & Co., New York (1998). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

The term “antibody” includes both polyclonal and monoclonal antibodies. The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

Typically, an antibody has a heavy and a light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody, whereas a V_{L} CDR1 is the CDR1 from the variable domain of the light chain of the antibody. As used herein, “V_{H}” refers to the variable region of an immunoglobulin heavy chain of an antibody, including the heavy chain of an Fv, scFv, or Fab. “V_{L}” refers to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

Furthermore, technologies have been developed for creating antibodies based on sequences derived from the human genome, for example by phage display or using transgenic animals (WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol., 222, 581-597; Knappik et al., J. Mol. Biol. 296: 57-86, 2000; S. Carmen and L. Jermutus, “Concepts in antibody phage display”. Briefings in Functional Genomics and Proteomics 2002 1(2):189-203; Lonberg N, Huszar D. “Human antibodies from transgenic mice”. Int Rev Immunol. 1995; 13(1):65-93.; Bruggemann M, Taussig M J. “Production of human antibody repertoires in transgenic mice”. Curr Opin Biotechnol. 1997 August; 8(4):455-8.). Such antibodies are “human antibodies” in the context of the present invention.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in an unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping is surface plasmon resonance.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

The phrase “binds specifically” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

Specific binding to an antibody under such conditions requires an antibody that is selected for its specificity for a particular protein. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W. H. Freeman and Co. 1976)).

Furthermore, a binding agent can interfere with the specific binding of a receptor, for example, an Lrp such as Lrp5 or Lrp6, and its ligand, for example, WISE, by various mechanism, including, for example, by binding to the ligand binding site, thereby interfering with ligand binding; by binding to a site other than the ligand binding site of the receptor, but sterically interfering with ligand binding to the receptor; by binding the receptor and causing a conformational or other change in the receptor, which interferes with binding of the ligand; or by other mechanisms. Similarly, the agent can bind to or otherwise interact with the ligand to interfere with its specifically interacting with the receptor. For purposes of the methods disclosed herein, an understanding of the mechanism by which the interference occurs is not required and no mechanism of action is proposed. An Lrp5 or Lrp6 antibody is characterized by having specific binding activity (Kₐ) for an Lrp5 or Lrp6 of at least about 10⁵ mol⁻¹, 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, Ann. N.Y. Acad. Sci. 51: 660-72, 1949).

As used herein, “modulate”, “modulates”, “modulating”, or other grammatical variations thereof means to change.

Non-limiting representative protein sequences for Lrp5 are listed in SEQ ID NOs.53-56, and the corresponding cDNAs are listed in SEQ ID NOs:383-386. Preferably, the Lrp5 is human (such as SEQ ID NO:54, a mature form or an isoform thereof). As used herein, the “mature form” of a protein means the form after post-translational processing, including the removal of signal sequences, which, in the case of the Lrps, are the approximately 20 N-terminal amino acids. As used herein “isoform” means an alternative form of a protein resulting from differential transcription of the relevant gene either from an alternative promoter or an alternate splicing site.

Non-limiting representative protein sequences for Lrp6 are listed in SEQ ID NOs:57-60, and the corresponding cDNAs are listed in SEQ ID NOs. 387-390. Preferably, the Lrp6 is human such as SEQ ID NO:58, a mature form or an isoform thereof).

Non-limiting representative protein sequences for WISE are listed in SEQ ID NOs:61-64, and the corresponding cDNAs are listed in SEQ ID NOs:391-394. Preferably, the WISE is human (such as SEQ ID NO:62, a mature form or an isoform thereof).

Non-limiting representative protein sequences for Lrp4 are listed in SEQ ID NOs:49-52, and the corresponding cDNAs are listed in SEQ ID NOs:379-382. Preferably, the Lrp4 is human such as SEQ ID NO:50, a mature form or an isoform thereof).

In one aspect of this embodiment, the antibody prevents binding between Lrp5 and WISE or Lrp6 and WISE. For example, the antibody may specifically bind to Lrp5 or Lrp6 at one or more sequences selected from the group consisting of SEQ ID NOs: 1-39, such as one or more of SEQ ID NOs: 1-15, one or more of SEQ ID NOs: 1-3 and 16-27, or one or more of SEQ ID NOs: 1-3 and 28-39. Preferably, the antibody binds specifically to Lrp5. In another preferred embodiment, the antibody binds specifically to Lrp6.

The details of SEQ ID NOs:1-39 are listed below.

(SEQ ID NO: 1)
HVTGASSSSSSSTK
(SEQ ID NO: 2)
ISLDTPDFTDIVLQ
(SEQ ID NO: 3)
VIIDQLPDLMGLKA
Lrp5
(SEQ ID NO: 16)
IEVANLNGTSRK
Lrp6
(SEQ ID NO: 28)
IEVSNLDGSLRK
Consensus
(SEQ ID NO: 4)
IEVXNLXGXXRK
Lrp5
(SEQ ID NO: 17)
AGAEEVLLLAR
Lrp6
(SEQ ID NO: 29)
DGATELLLLAR
Consensus
(SEQ ID NO: 5)
XGAXEXLLLAR
Lrp5
(SEQ ID NO: 18)
ISLDTPDFTDIVLQVDDIR
Lrp6
(SEQ ID NO: 30)
ISLDTPDFTDIVLQLEDIR
Consensus
(SEQ ID NO: 6)
ISLDTPDFTDIVLQXXDIR
Lrp5
(SEQ ID NO: 19)
ANLDGQERR
Lrp6
(SEQ ID NO: 31)
AALDGSDRV
Consensus
(SEQ ID NO: 7)
AXLDGXXRX
Lrp5
(SEQ ID NO: 20)
ISLETNNNDVAIPLTGVK
Lrp6
(SEQ ID NO: 32)
ISLETNNNNVAIPLTGVK
Consensus
(SEQ ID NO: 8)
ISLETNNNXVAIPLTGVK
Lrp5
(SEQ ID NO: 21)
EASALDFDVSNNH
Lrp6
(SEQ ID NO: 33)
EASALDFDVTNDR
Consensus
(SEQ ID NO: 9)
EASALDFDVXNXX
Lrp5
(SEQ ID NO: 22)
IYWTDVSLKT
Lrp6
(SEQ ID NO: 34)
IYWTDISLKT
Consensus
(SEQ ID NO: 10)
IYWTDXSLKT
Lrp5
(SEQ ID NO: 23)
AIVVNAER
Lrp6
(SEQ ID NO: 35)
AVVVNPEK
Consensus
(SEQ ID NO: 11)
AXVVNXEX
Lrp5
(SEQ ID NO: 24)
RIESCDLSGANR
Lrp6
(SEQ ID NO: 36)
RIESSDLSGANR
Consensus
(SEQ ID NO: 12)
RIESXDLSGANR
Lrp5
(SEQ ID NO: 25)
CASGQCVLI
Lrp6
(SEQ ID NO: 37)
CANGQCIGK
Consensus
(SEQ ID NO: 13)
CAXGQCXXX
Lrp5
(SEQ ID NO: 26)
CDSFPDCIDG
Lrp6
(SEQ ID NO: 38)
CDHNVDCSDK
Consensus
(SEQ ID NO: 14)
CDXXXCDXDX
Lrp5
(SEQ ID NO: 27)
NHVTGASSSSSSSTK
Lrp6
(SEQ ID NO: 39)
AHVTGASSSSSSSTK
Consensus
(SEQ ID NO: 15)
XHVTGASSSSSSSTK

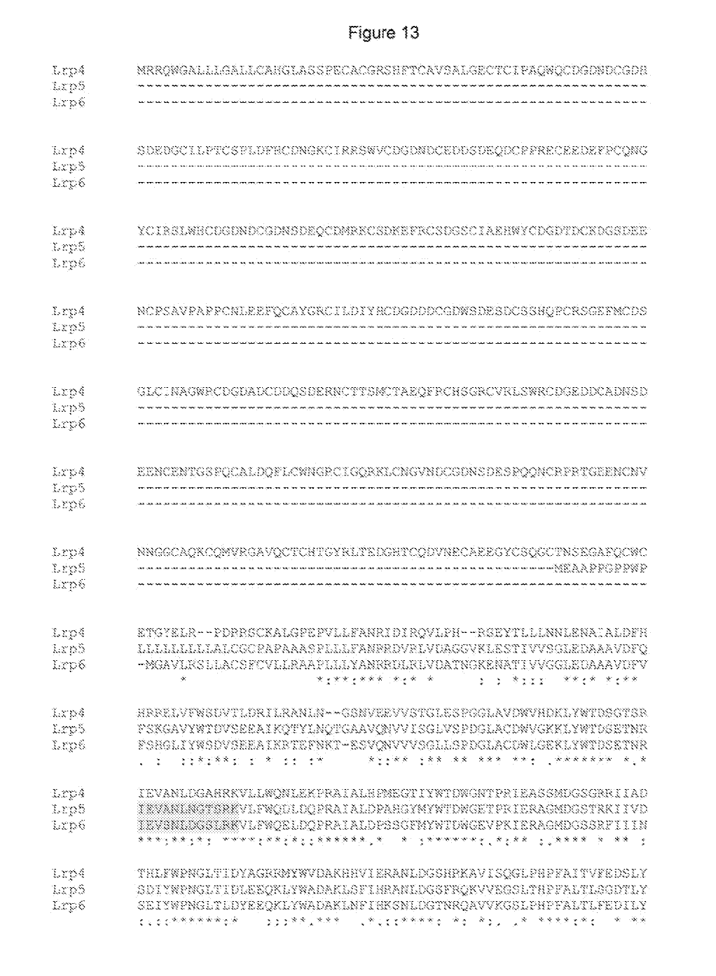

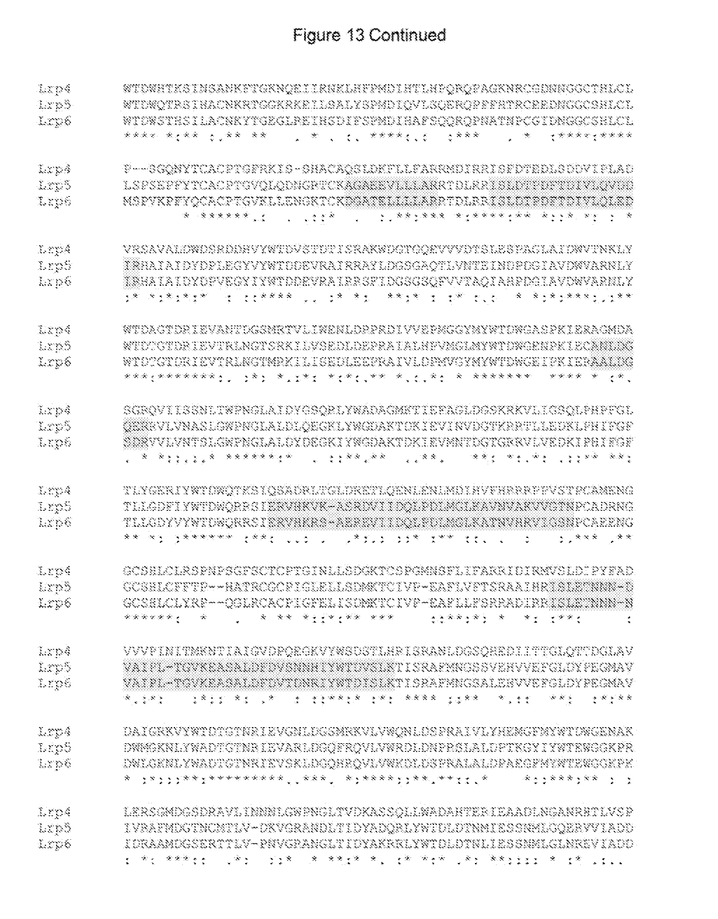

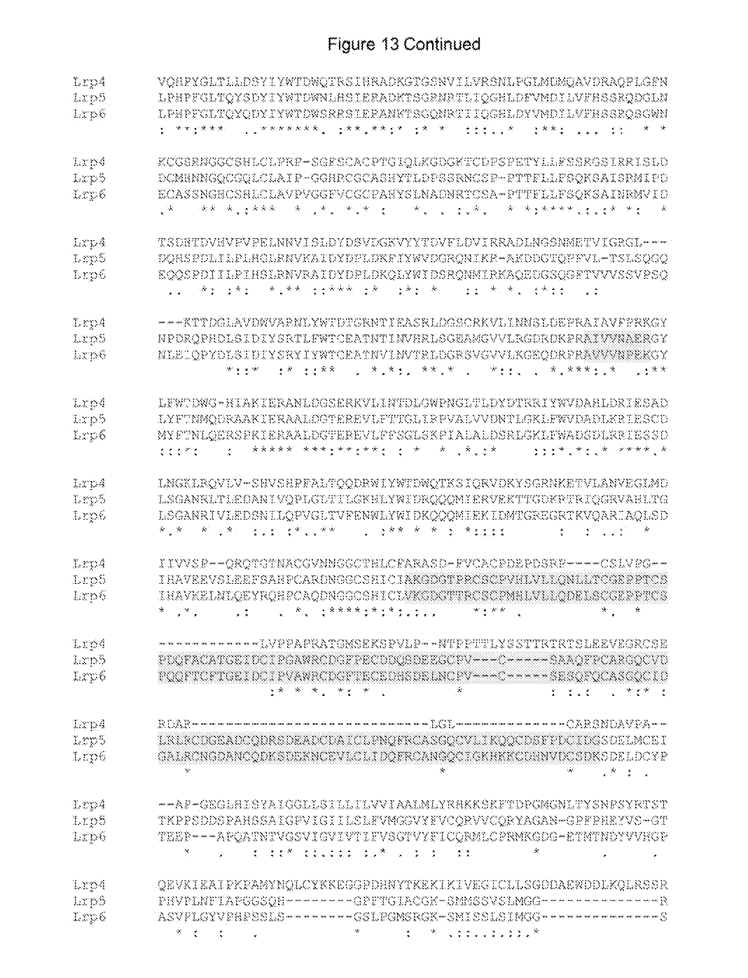

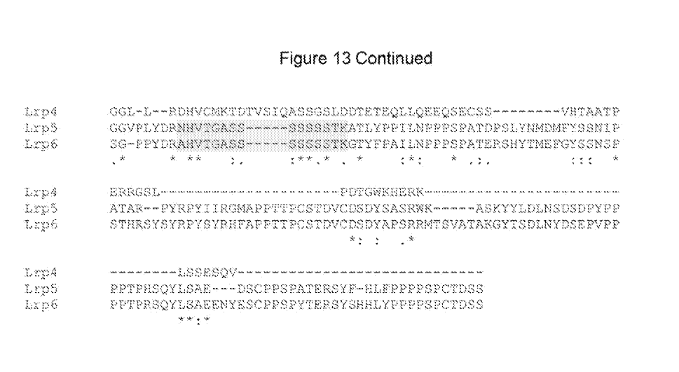

In another aspect of this embodiment, the antibody specifically binds to Lrp5 or Lrp6 within a sequence selected from the group consisting of SEQ ID NOs:40-48. For example, such an antibody may specifically bind to Lrp5 or Lrp6 at SEQ ID NOs:65-378. It is noted that SEQ ID NOs: 46 and 47 contain the amino acid sequences just before the transmembrane domain of Lrp5 and Lrp6, respectively. The identified portions of the Lrp5 and Lrp6 are very similar to each other, but are very different from the sequence of Lrp4 (see FIG. 13).

The details of SEQ ID NOs:40-48 are listed below.

Lrp5
(SEQ ID NO: 40)
ERVHKVKASRDVIIDQLPDLMGLKAVNVAKVVGTN
Lrp6
(SEQ ID NO: 41)
ERVHKRSAEREVIIDQLPDLMGLKATNVHRVIGSN
Consensus
(SEQ ID NO: 42)
ERVHKXXAXRXVIIDQLPDLMGLKAXNVXXVXGXN
Lrp5
(SEQ ID NO: 43)
RISLETNNNDVAIPLTGVKEASALDFDVSNNHIYWTDVSLKT
Lrp6
(SEQ ID NO: 44)
RISLETNNNNVAIPLTGVKEASALDFDVTDNRIYWTDISLKT
Consensus
(SEQ ID NO: 45)
RISLETNNNXVAIPLTGVKEASALDFDVXXNXIYWTDXSLKT
Lrp5
(SEQ ID NO: 46)
KGDGTPRCSCPVHLVLLQNLLTCGEPPTCSPDQFACATGEIDCIPGA
WRCDGFPECDDQSDEEGCPVCSAAQFPCARGQCVDLRLRCDGEADCQD
RSDEADCDAICLPNQFRCASGQCVLIKQQCDSFPDCIDGSDEL
Lrp6
(SEQ ID NO: 47)
KGDGTTRCSCPMHLVLLQDELSCGEPPTCSPQQFTCFTGEIDCIPVA
WRCDGFTECEDHSDELNCPVCSESQFQCASGQCIDGALRCNGDANCQDK
SDEKNCEVLCLIDQFRCANGQCIGKHKKCDHNVDCSDDEL
Consensus
(SEQ ID NO: 48)
KGDGTXRCSCPXHLVLLQXXLXCGEPPTCSPXQFXCXTGEIDCIPXA
WRCDGFXECXDXSDEXXCPVCSXXQFXCAXGQCXDXXLRCXGXAXCQDX
SDEXXCXXLCLXXQFRCAXGQCXXXXXXCDXXXDCXDXSDEL

In another aspect of this embodiment, Lrp4, Lrp5, and Lrp6 are human.

In an additional aspect of this embodiment, the antibody is monoclonal.

In a further aspect of this embodiment, the antibody is human, humanized, or chimeric.

Another embodiment of the present invention is a pharmaceutical composition. This pharmaceutical composition comprises an antibody of the present invention such as, e.g., an antibody that modulates binding between Lrp5 and WISE or Lrp6 and WISE, but does not modulate binding between Lrp4 and WISE, and at least one pharmaceutically acceptable excipient or diluent.

A further embodiment of the present invention is a method for preventing WISE binding to Lrp5 or Lrp6, but not WISE binding to Lrp4, comprising contacting Lrp5 or Lrp6 with an agent that binds to Lrp5 or Lrp6 but not Lrp4.

A binding agent according to the present invention may be an antibody, or non-immunoglobulin “antibody mimics”, sometimes called “scaffold proteins”, may be based on the genes of protein A, the lipocalins, a fibronectin domain, an ankyrin consensus repeat domain, and thioredoxin (Skerra, Current Opinion in Biotechnology 2007, 18(4): 295-304). A preferred embodiment in the context of the present invention are designed ankyrin repeat proteins (DARPin's; Steiner et al., J Mol Biol. 2008 Oct. 24; 382(5): 1211-27; Stumpp M T, Amstutz P. Curr Opin Drug Discov Devel. 2007 March; 10(2):153-9). Preferably, the agent is an antibody. More preferably, the antibody is monoclonal.

In one aspect of this embodiment, the agent is an antibody that specifically binds to Lrp5 or Lrp6. Preferred binding sites are as disclosed herein.

An additional embodiment of the present invention is a method for preventing human WISE from binding to human Lrp5 or human Lrp6 comprising contacting human WISE with a monoclonal antibody, which antibody specifically binds to human Lrp5 or human Lrp6 but not to human Lrp4. Preferred binding sites are as disclosed herein.

A further embodiment of the present invention is a kit. This kit comprises one or more of the antibodies of the present invention.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of antibody in the compositions disclosed herein is from about 0.1 mg/kg to about 150 mg/kg per day, such as from about 0.5 mg/kg to about 50 mg/kg per day, including from about 1 mg/kg to about 100 mg/kg per day. Other representative dosages of such agents include about 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, and 150 mg/kg per day. The effective dose of antibody in the compositions disclosed herein maybe administered as two, three, four, five, six or more sub-doses, administered separately at appropriate intervals throughout the day.

A composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a composition of the present invention may be administered in conjunction with other treatments. A composition of the present invention maybe encapsulated or otherwise protected against gastric or other secretions, if desired.

The compositions of the invention comprise one or more active ingredients i.e., antibodies of the present invention, in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

Pharmaceutical compositions may be prepared by mixing the antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, pchlor-m-cresol, methyl or propyl parabens, benzalkonium chloride or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-Methylglucosamine (so-called “Meglumine”), galactosamine and neuraminic acid; and/or non-ionic surfactants such as Tween, Brij Pluronics, Triton-X or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization), however, solutions comprising antibacterial agents also may be used for the production of pharmaceutical compositions for parenteral administration; see also Chen (1992) Drug Dev Ind Pharm 18, 1311-54.

Exemplary antibody concentrations in the pharmaceutical composition may range from about 1 mg/mL to about 200 mg/ml or from about 50 mg/mL to about 200 mg/mL, or from about 150 mg/mL to about 200 mg/mL. For clarity reasons, it is emphasized that the concentrations as indicated herein relate to the concentration in a liquid or in a liquid that is accurately reconstituted from a solid form.

An aqueous formulation of the antibody may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the antibody formulation to modulate the tonicity of the formulation. Exemplary tonicity agents include sodium chloride, potassium chloride, glycerin and any component from the group of amino acids, sugars as well as combinations thereof. Preferably, the aqueous formulation is isotonic, although hypertonic or hypotonic solutions may be suitable. The term “isotonic” denotes a solution having the same tonicity as some other solution with which it is compared, such as a physiological salt solution and the blood serum. Tonicity agents may be used in an amount of about 5 mM to about 350 mM, in particular in an amount of 105 mM to 305 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Preferred polyoxyethylenesorbitan-fatty acid esters are polysorbate 20, (sold under the trademark Tween 20™) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants are generally used in an amount of about 10 mM to 500 nM.

In one embodiment, the formulation contains the above-identified agents (i.e. antibody, surfactant, buffer, stabilizer and/or tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In another embodiment, a preservative may be included in the formulation, e.g., at concentrations ranging from about 0.001 to about 2% (w/v).

Compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions of the present invention for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Compositions of the present invention which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods of the present invention. These examples are illustrative only and are not intended to limit the scope of the invention in any way.

### EXAMPLES

### Example 1

### Materials and Methods

### Mouse Strains

Lrp4^{mdig}, Lrp4^{mitt}, Lrp4^{mte}, Wise, TopGal, Lrp5, Lrp6, Ctnnb1^{fx}, K14cre, Ptch1^{Lacz} and R26-floxstop-LacZ mice were described previously (DasGupta and Fuchs, 1999; Milenkovic et al., 1999; Soriano, 1999; Dassule et al., 2000; Pinson et al., 2000; Brault et al., 2001; Kato et al., 2002; Simon-Chazottes et al., 2006; Weatherbee et al., 2006; Ahn et al., 2010; Ferrer-Vaguer et al., 2010). All experiments involving mice were approved by the Institutional Animal Care and Use Committee of the Stowers Institute for Medical Research (Protocol 2010-0062).

Generation of Lrp4-LacZ, K14-tTA , TCF-tTA and tetO-Wise Transgenic Mice

For Lrp4-LacZ BAC reporter, a mouse BAC clone, RP23-276H15, was modified to contain an 134 kb genomic region which covers the whole Lrp4 coding region and neighboring upstream (36 kb) and downstream (44 kb) sequences using the bacterial recombination technology (Lee et al., 2001). LacZ was then inserted in-frame into the first coding exon of Lrp4. The K14-tTA was generated by inserting the K14 promoter (Ahn et al., 2010) and a synthetic intron (IVS) (Clontech) upstream of VP22-tTA-SV40pA (Gossen and Bujard, 1992). For TCF-tTA, the K14 promoter of K14-tTA was removed except the basal promoter region (−120 to +13) and replaced with the multiple TCF binding sites from TOPFLASH vector (Millipore). To make tetO-Wise, G-CaMP2 of tetO-G-CaMP2 (He et al., 2008) was replaced with a Wise ORF, and then IRES-eGFP was subcloned between Wise and SV40pA. Transgenic founders were generated by pro-nuclear injection of linearized constructs into C57BI/10J xCBA-F1 embryos.

### Generation of Wise-LacZ and K14-Wise Transgenic Mice

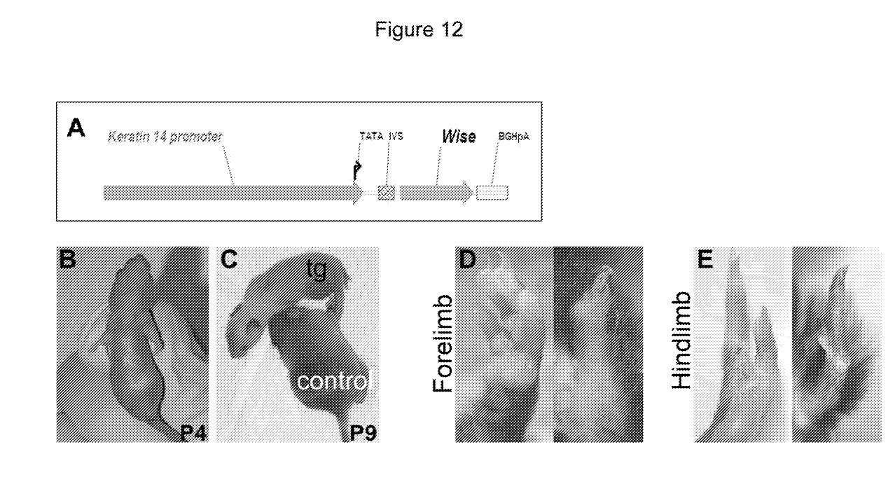

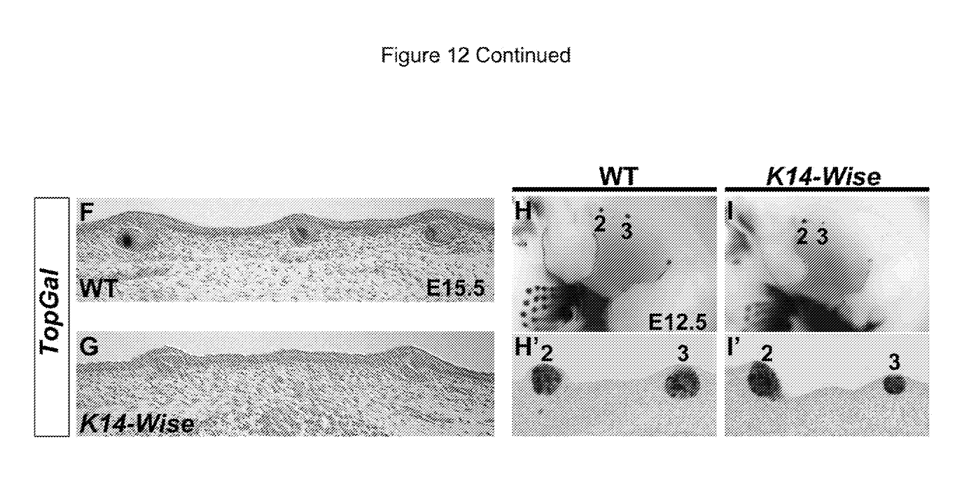

The Wise-LacZ construct was generated by inserting a LacZ-SV40pA in-frame into the first coding exon of Wise in the 24 kb EcoR I-Sal I genomic fragment from a mouse BAC clone, RP23-98E22. Three of five Wise-LacZ lines mimicked the known expression pattern of Wise in the skin appendages and used for this study (FIG. 12).

The K14-Wise construct has been previously described (Ahn et al., 2010). Two of the 9 K14-Wise transgenic founders showed severely deformed limbs and hair loss (data not shown). By in vitro fertilization using sperm from one of the founders, transgenic progeny which phenocopied their parents were generated (FIG. 12). The K14-Wise transgene also was injected into eggs harvested from TopGal females to monitor changes in Wnt signaling (FIG. 12).

### β-Gal Staining, In Situ Hybridization and BrdU Analysis

To detect β-Galactosidase activity, embryos were fixed in either 0.1% paraformaldehyde/0.2% glutaraldehyde (E11.5-E13.5) or 4% paraformaldehyde (PFA) (E14.0 or older) for 30-60 minutes on ice. After several washes in phosphate buffered saline, samples were stained in X-Gal for 4-20 hours at 4° C. or at room temperature. Whole-mount in situ hybridization was performed with embryos fixed in 4% PFA overnight according to standard protocols using DIG-labeled anti-sense riboprobes. Histological samples were paraffin-embedded after post-fixation in 4% PFA, sectioned at 8 μm and counterstained with nuclear fast red. For analysis of cell proliferation and cell death, embryos were harvested 2 hours after intraperitoneal injection of BrdU (50 μg/g body weight) into pregnant females, sectioned and stained with a mouse anti-BrdU antibody (Amersham), a mouse E-cadherin antibody (BD Biosciences), or a rabbit caspase 3 antibody (Cell Signaling).

### Confocal Microscopy and Cell Counting

Fluorescent images were obtained by the LSM 710 confocal microscope (Carl Zeiss). Nuclei with fluorescence above basal level were counted using the Imaris software (Bitplane).

### Example 2

### Abnormal Development of the Mammary Glands in Lrp4 Mutant Mice

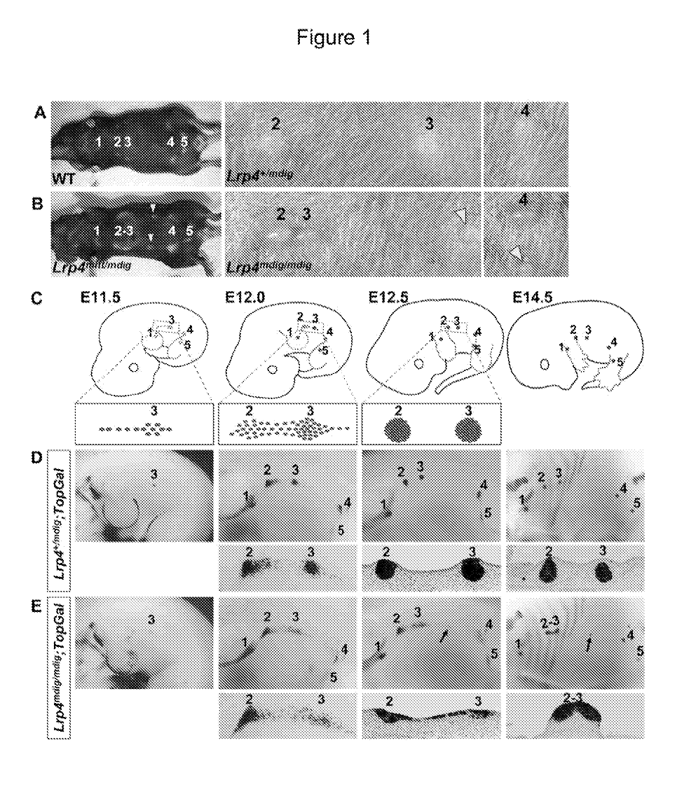

Lrp4 is known to be expressed in placodes of skin appendages such as mammary glands, hair follicles and vibrissae (Weatherbee et al., 2006; Fliniaux et al., 2008). Thus, potential roles for Lrp4 in development of these tissues were examined. Mice homozygous for null alleles of Lrp4 (Lrp4^{mitt} and Lrp4^{mte}) die after birth, but mice homozygous for a hypomorphic allele (Lrp4^{mdig}) survive to reach adulthood (Simon-Chazottes et al., 2006; Weatherbee et al., 2006). The analyses of both Lrp4^{mitt/mdig} and Lrp4^{mdig/mdig} females revealed a variety of abnormalities in the number, position and morphology of nipples (FIGS. 1A and 1B and data not shown). In Lrp4 mutant females, nipples #2 and 3 were frequently fused and the individual nipples were enlarged compared to those of control females. In addition, ectopic nipples were present in the region between nipples #3 and #4 and around nipple #4 (yellow arrowheads in FIG. 1B). The ectopic nipples were smaller than normal nipples and were associated with little or no fat pads, suggesting that they are non-functional (Data not shown).

### Example 3

### Lrp4 is Essential for Patterning of the Mammary Placodes

The mammary defects in Lrp4 mutants suggest that Lrp4 plays a role in embryonic mammary development. The number and position of the nipples and associated mammary glands is primarily determined around embryonic day 12 (E12) when the mammary placodes develop (Cowin and Wysolmerski, 2010). The TopGal reporter mouse line (DasGupta and Fuchs, 1999) was used to follow the progress of mammary development and to monitor changes in the activity of Wnt/β-catenin signaling (FIGS. 1C-1E). Consistent with a previous report (Chu et al., 2004), in control embryos, TopGal expressing epithelial cells were spread along the mammary lines at E11.5, and within a day they sequentially became restricted to placodes in a defined order (#3, 1/4, 5, and finally 2). TopGal expression was gradually lost in the inter-placodal regions and after E12.5, TopGal expression was seen only in the epithelial cells of the mammary buds.

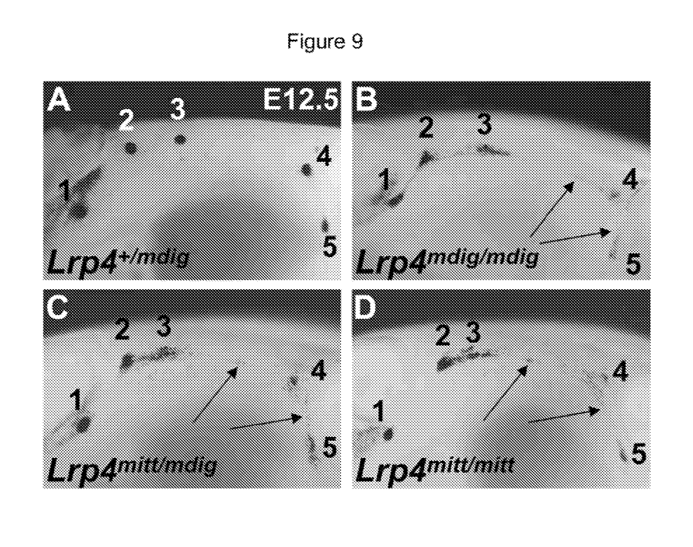

In Lrp4^{mdig/mdig} embryos, TopGal expressing cells were more loosely organized around the developing placodes at E12.0 suggesting that placode assembly was delayed (compare FIG. 1D with 1E). This is particularly apparent in placodes # 3 and 4 at this stage. Consistent with a delay, the mutant placodes displayed broader but shallower epithelial invagination at E12.5, which is typical of an earlier stage placode. Furthermore, there were ectopic TopGal-expressing cells spread along the mammary line. A large proportion of these cells were found around the underdeveloped placodes, especially placodes #2, 3 and 4. It appeared that some of these cells later give rise to supernumerary placodes (arrows in FIG. 1E) in the interplacodal region, which corresponds to the site of supernumerary nipples in adults. Similar changes in the TopGal expression pattern were observed in Lrp4^{mitt/mdig} and Lrp4^{mitt/mitt} mice, indicating that the observed defects are loss-of-function phenotypes (FIG. 9). These data indicate that Lrp4 is essential for mammary patterning by facilitating assembly of the placodes and controlling the distribution and number of the mammary epithelial cells.

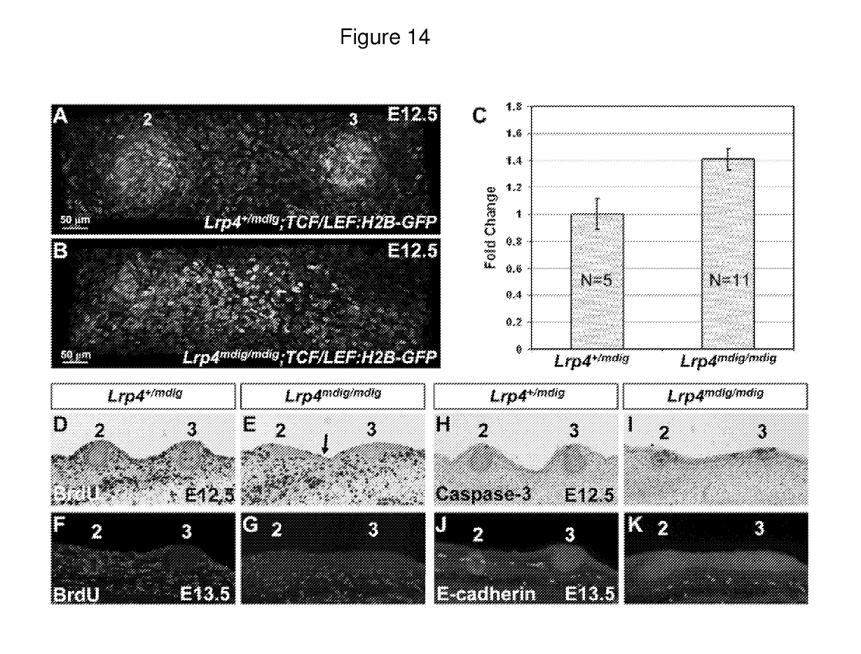

Whether the abnormal mammary patterning in Lrp4 mutants is associated with changes in the number of mammary epithelial cells was investigated using the TCF/LEF:H2B-GFP reporter, which marks mammary placodes similar to TopGal (Ferrer-Vaquer et al., 2010). Confocal imaging of the placode 2/3 region revealed a 40% increase in the total number of GFP-expressing cells (FIGS. 14A-C). Together, these data indicate that Lrp4 is required for facilitating the assembly of mammary placodes and for limiting the number of mammary epithelial cells.

### Example 4

### Reduced Proliferation of the Misplaced Mammary Epithelial Cells Causes Placode Fusion

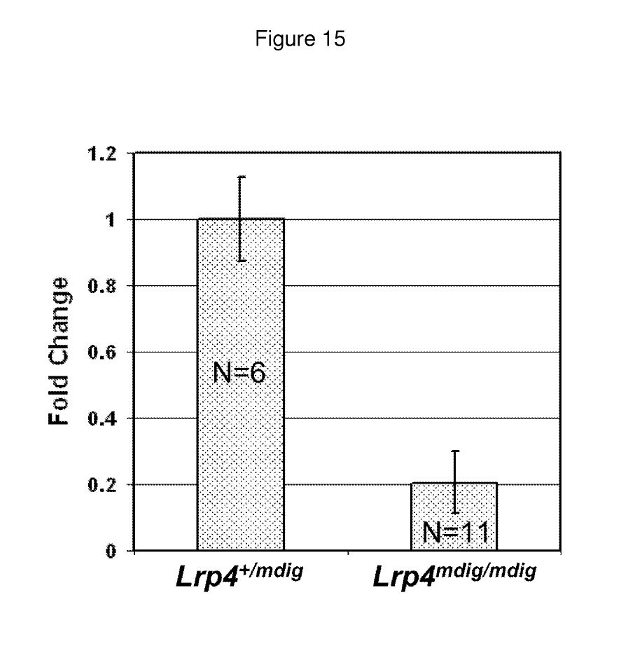

Although mammary placodes #2 and 3 were developmentally delayed and morphologically abnormal in Lrp4 mutants, they were centered at fairly normal positions at E12.0 (FIG. 1E). However, afterwards the distance between the two placodes was reduced compared to controls, leading to fusion later in development (FIG. 1E). To investigate the underlying basis of this placode fusion, the rate of cell proliferation and cell death was examined. As previously reported (Balinsky, 1950), in control mice, non-mammary epithelial cells surrounding the placodes were actively proliferating while the placodes themselves displayed a very low level of proliferation (FIG. 14D). In contrast, in Lrp4^{mdig/mdig} mice, cell proliferation was greatly reduced in the interplacodal region (FIGS. 14E and 15). The interplacodal region continued to show reduced proliferation and was thickened at E13.5 in the mutants (FIGS. 14F, 14G, 14J, and 14K). Combined with the TopGal and TCF/LEF:H2BGFP expression data, the results indicate that cells in the interplacodal region in Lrp4 mutants possess mammary fate.

With respect to cell death, in control mice, a small number of apoptotic cells were observed mostly around the neck of the buds, but not in the interplacodal epithelium (FIG. 14H). In Lrp4 mutants, more apoptotic cells were observed in the interplacodal region and also around the sites of invagination (FIG. 14I). Together, these results suggest that placode fusion in the mutants is largely due to relatively slow growth of ectopic mammary epithelial cells in the interplacodal region that forms part of the large extended placode, but removal of some epithelial cells by cell death also contributes to the fusion.

### Example 5

### Reduction in the Dosage of the Lrp5 And Lrp6 Wnt Co-Receptors Ameliorates the Lrp4 Mutant Defects in Limb and Mammary Patterning

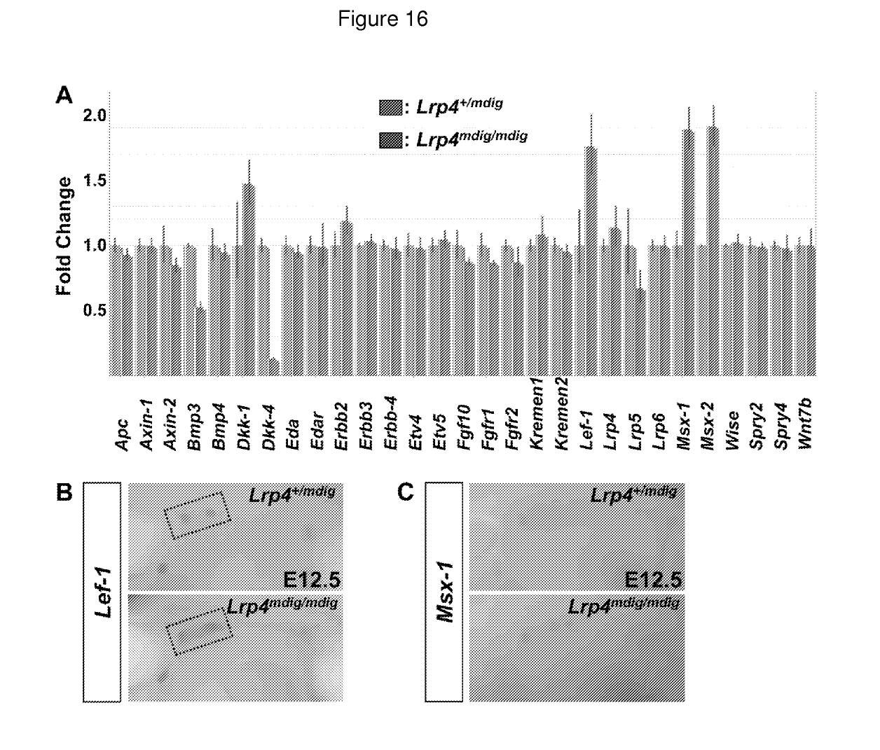

To examine which signaling pathways were misregulated in the mammary placodes of Lrp4 mutants, placodes 2 and 3 were dissected from E12.5 embryos and expression analysis was performed using qPCR assays designed for components of Wnt, FGF, TGFβ/BMP and Eda pathways (FIG. 16). Differential expression of genes in Wnt (Dkk1, Dkk4 and Lef1) and TGFβ/BMP (Bmp3, Msx1 and Msx2) pathways suggests that signaling activity of the two pathways is changed in Lrp4 mutants.

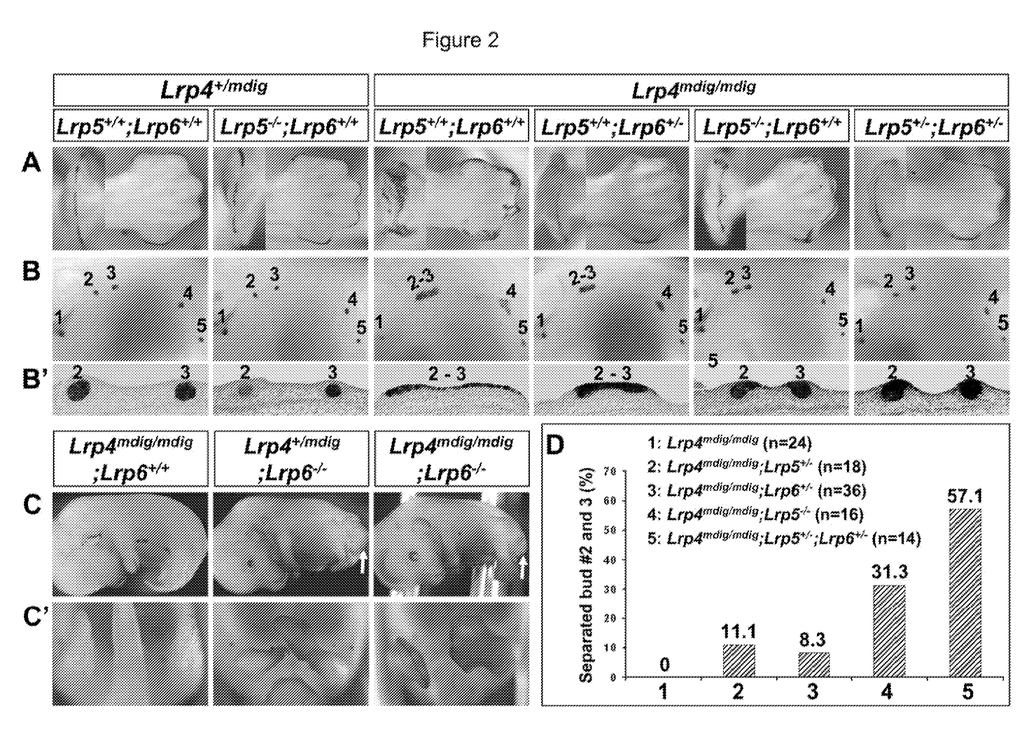

The increased number and abnormal distribution of cells expressing the Wnt reporters (FIGS. 1 and 14) and Lef-1 (FIG. 16) in Lrp4 mutants raise the possibility that misregulation of Wnt/β-catenin signaling is causally related to the mammary abnormalities. To explore this idea, genetic interactions between Lrp4 and the Wnt co-receptor genes, Lrp5 and Lrp6, were examined. In crosses between Lrp4 and Lrp5/6 mutants, limb defects were first focused on as a means to score for genetic interactions (FIG. 2A). All the known Lrp4 mutants have been characterized by abnormal patterning of the apical ectodermal ridge (AER) and polysyndactyly, while Lrp5;Lrp6 compound mutants displayed limb defects in a dose dependent manner (Holmen et al., 2004; Johnson et al., 2005; Simon-Chazottes et al., 2006; Weatherbee et al., 2006). At E13.5, TopGal expressing cells were normally confined to AER as a thin line, but in Lrp4 mutants these cells were scattered in the distal limb buds due to broadening of AER. Interestingly, inactivating two copies of Lrp5 or a single copy of Lrp6 ameliorated the AER defects of Lrp4 mutants and fairly normal limb patterning was observed in Lrp4^{mdig/mdig};Lrp5^{+/-};Lrp6^{+/-} mice. Loss of Lrp6 resulted in severe limb defects with a stronger effect on hind limbs (Pinson et al., 2000; Zhou et al., 2010). Such limb defects of Lrp6-null mice were significantly rescued in Lrp4^{mdig/mdig};Lrp6^{-/-} mice (n=5) (FIG. 2C,C′) indicating that Lrp4 and Lrp6 act antagonistically.

It has been shown that embryonic mammary development is delayed or severely impaired in Lrp5^{-/-} and Lrp6^{-/-} mice, respectively, in association with reduced Wnt signaling activity. (Lindvall et al., 2006; Lindvall et al., 2009). Therefore, whether reduced doses of Lrp5 and Lrp6 can rescue the mammary defects of Lrp4 mutants was investigated (FIGS. 2B and 2B′). Lrp5^{-/-} mice displayed reduction in placode size as previously reported. When both copies of Lrp5 or a single copy of Lrp6 were inactivated in Lrp4 mutants, TopGal expressing cells were more confined around the sites of bud formation indicating amelioration of Lrp4 mutant phenotypes. Furthermore, in Lrp4^{mdig/mdig};Lrp5^{+/-};Lrp6^{+/-} mice, the buds appeared to be fairly normal and buds #2 and 3 were fully separated in the majority of cases (FIG. 2D). These genetic interactions indicate that the abnormal limb and mammary development in Lrp4 mutants is largely due to elevated Wnt signaling and support the idea that Lrp4 inhibits Wnt/β-catenin signaling in vivo.

### Example 6

### Lrp4 Facilitates Placode Formation and Restricts Mammary Fate by Inhibiting Wnt/B-Catenin Signaling

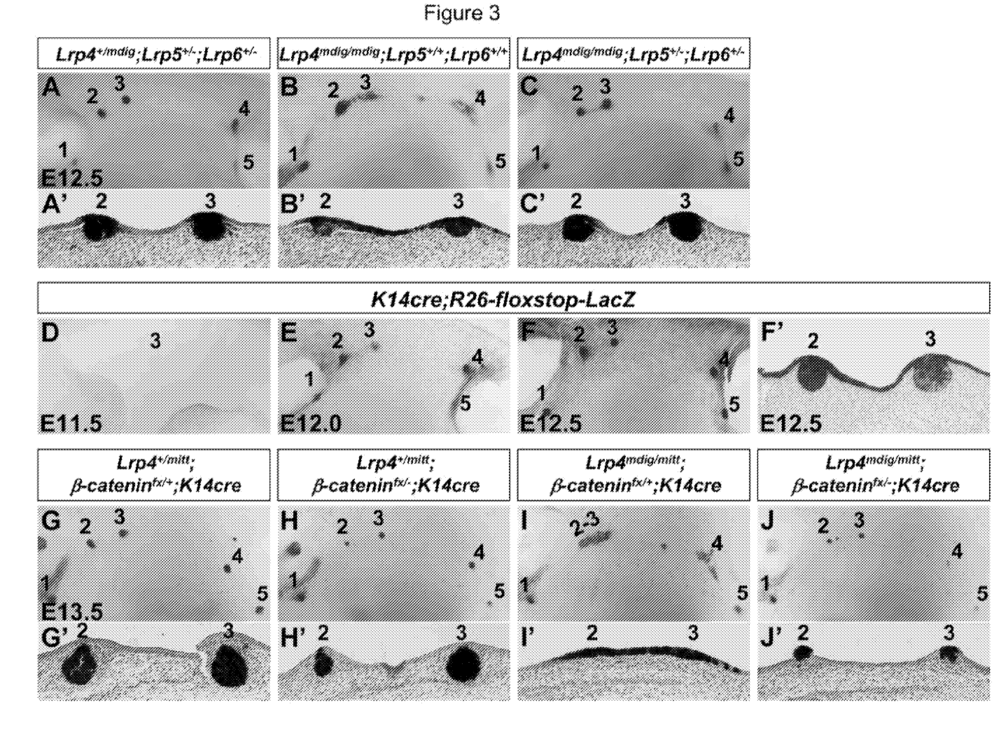

Whether the normal timing of placode initiation is restored in Lrp4^{mdig/mdig};Lrp5^{+/-};Lrp6^{+/-} mice was investigated. Indeed, the compound mutants displayed placodes with almost normal morphology and size with few TopGal-expressing cells in the interplacodal region at E12.5 (FIG. 3A-3C and 3A′-3C′). This suggests that a reduction in Wnt/β-catenin signaling can compensate for loss of Lrp4 function and facilitate placode formation in Lrp4 mutants.

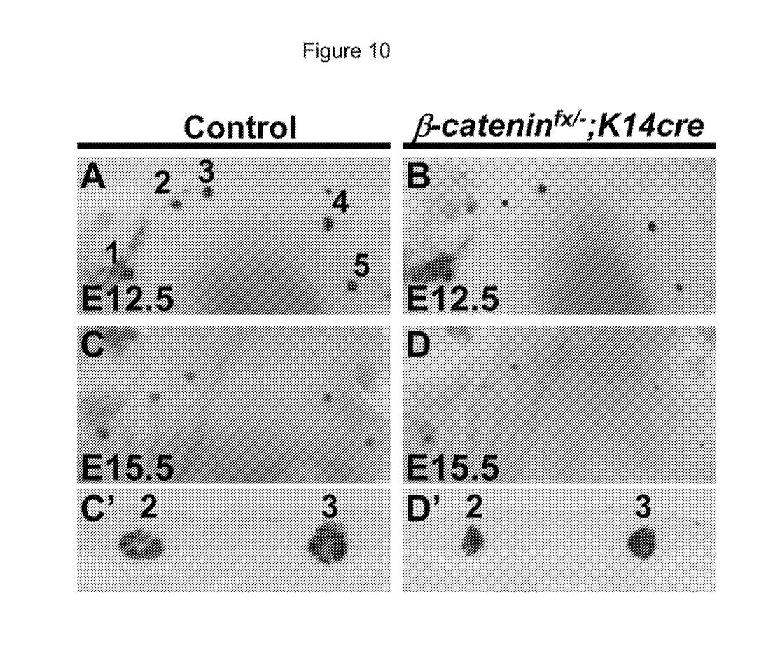

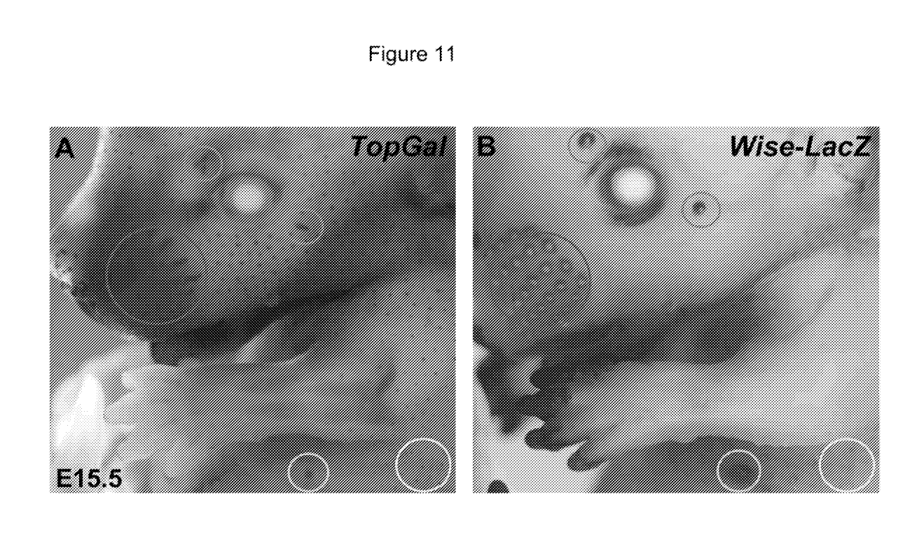

To further explore a role for Wnt/β-catenin signaling in controlling the number of mammary epithelial cells, the β-catenin gene (Ctnnb1) was inactivated in the epithelium after placode initiation using a conditional allele of β-catenin combined with a Cre line driven by a Keratin 14 promoter (K14cre). K14cre can induce recombination in a subset of epithelial cells along the mammary line at E11.5-E12.0 (FIGS. 3D and 3E). By E12.5, Cre activity is detected in most epithelial cells in and around the mammary buds (FIGS. 3F and 3F′). In β-catenin^{fx/-};K14cre mice, all the buds formed at their normal position consistent with the late onset of Cre activity (FIGS. 3G, 3H, 3G′, and 3H′), but they were smaller at E12.5 and remained growth-retarded afterwards (FIG. 10). This suggests that Wnt/β-catenin signaling is required for producing a sufficient pool of the mammary precursor cells and for facilitating growth of the buds at later stages.

Whether inactivation of β-catenin has effects on the Lrp4 mutant phenotypes was tested (FIGS. 3I, 3J, 3I′, and 3J′). Interestingly, a greater reduction in placode size was observed in Lrp4 mutants than in control mice when β-catenin was inactivated. Lrp4^{mdig/mdig}; β-catenin-^{-/fx};K14cre mice often developed a variable number of small placodes in the placodes #2 and 3 region. This was interpreted to mean that due to the delay in placode formation in Lrp4 mutants, β-catenin was inactivated at a relatively earlier stage of placode development, resulting in further reduction in mammary precursor cells. These genetic analyses further support the idea that Wnt/β-catenin is also essential for inducing or maintaining the mammary fate in the epithelial cells before placode assembly. Taken together, these data suggest that Lrp4 normally facilitates placode formation and limits the number of mammary epithelial cells by inhibiting Wnt/β-catenin signaling.

### Example 7

### Lrp4 is Required for Development of Hair and Vibrissal Follicles

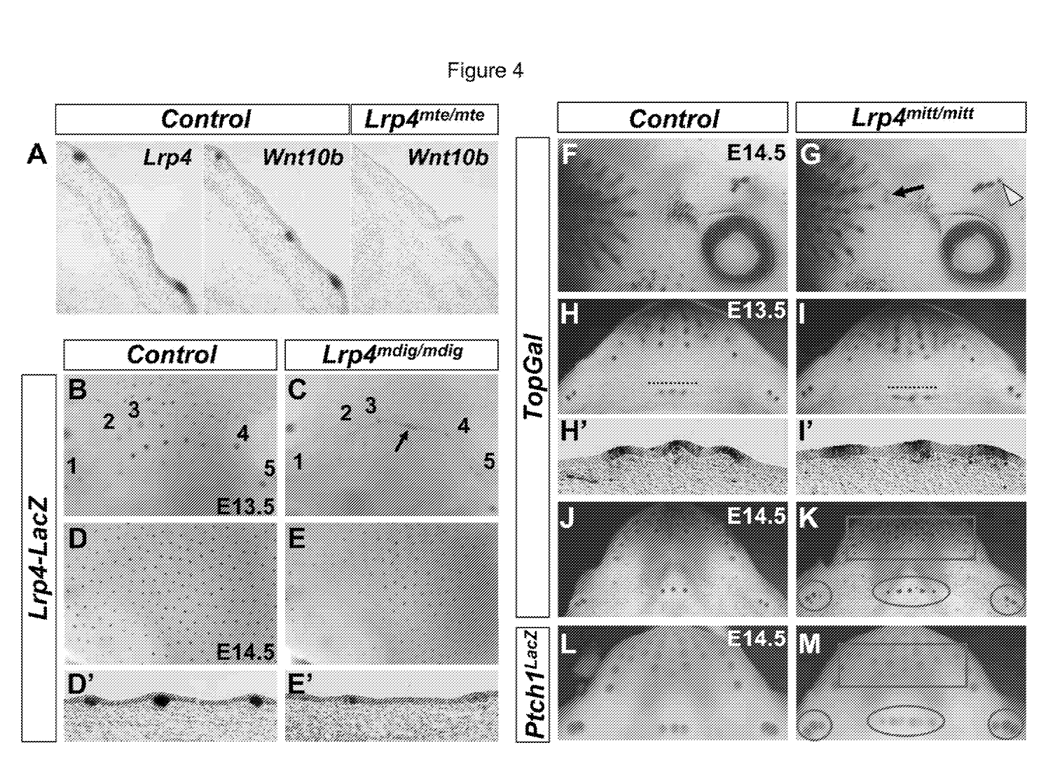

Whether the findings in mammary gland development reflect related roles for Lrp4 in other skin appendages was investigated. Primary hair follicles were marked by Wnt10b transcripts at E14.5 in control mice, but in the Lrp4 mutant skin, Wnt10b was undetectable (FIG. 4A). The Lrp4-LacZ BAC reporter line marked newly forming hair placodes at E13.5 and continued to express in the primary hair follicles (FIGS. 4B and 4C), mimicking endogenous Lrp4 expression pattern (Fliniaux et al., 2008). In Lrp4 mutants, Lrp4-LacZ expression was not observed at E13.5, and hair placodes were less developed compared to those of control mice at E14.5 (FIGS. 4C and 4E), indicating that hair follicle development is delayed.

Groups of vibrissae develop in different regions of the mouse head (Yamakado and Yohro, 1979), and supernumerary vibrissal follicles were observed for each group in Lrp4 mutants (FIGS. 4H-I and 11). In particular, extra interramal vibrissal follicles which form along a transverse line under the chin at E14.5 were detected (FIGS. 4J-4M). One day earlier, there was a delay in morphogenesis of the follicles in Lrp4 mutants with less condensed domains of TopGal expression (FIGS. 4H, 4I, 4H′, 4I′). In general, these phenotypes were milder and less penetrant in Lrp4^{mdig/mdig} mice compared to Lrp4^{mitt/mitt} mice (data not shown). The analyses revealed that Lrp4 is required for timely formation of hair and vibrissal follicles and suggest that Lrp4 normally facilitates morphogenesis of these skin placodes, similar to its role in mammary placodes.

### Example 8

### Wise is Required for Development of Mammary Glands and Vibrissae

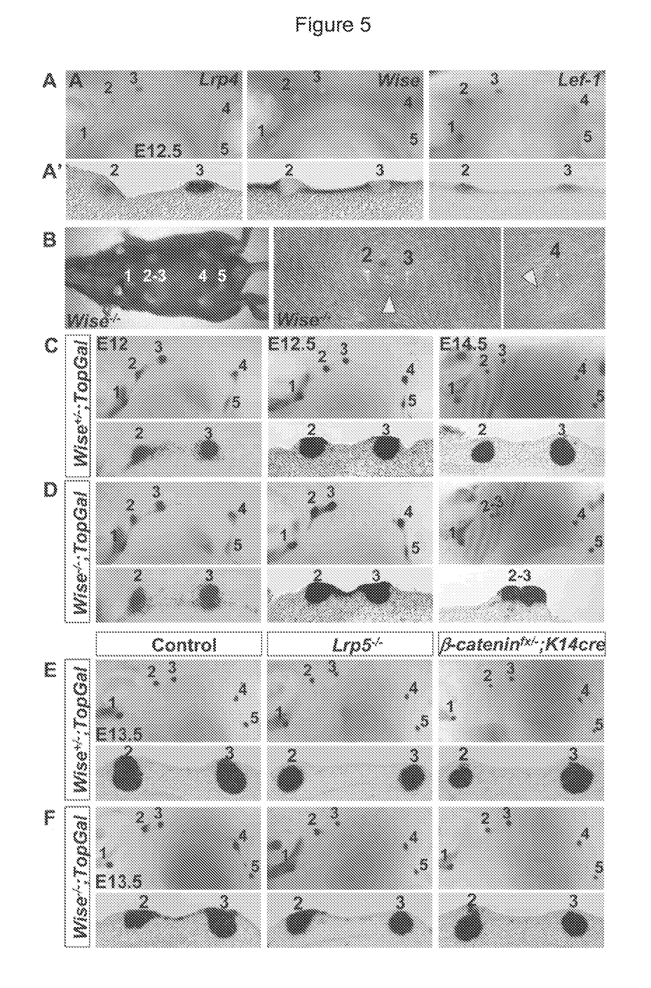

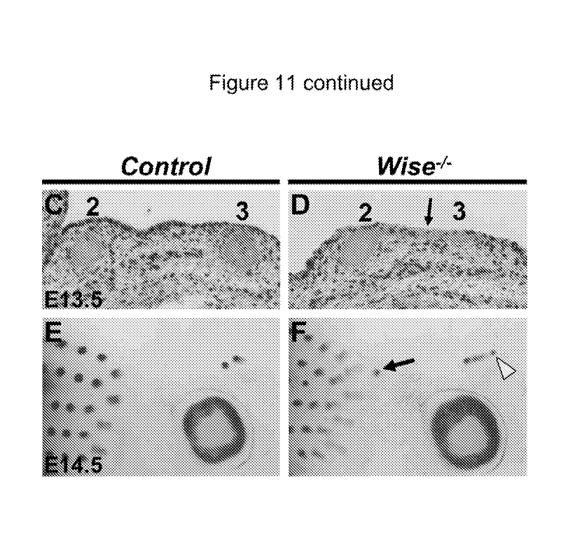

Because Wise is a potential ligand for Lrp4 and mice deficient for Wise or Lrp4 displayed similar tooth defects, roles for Wise in the mammary glands and other skin appendages were investigated. Earlier studies have shown that in developing skin appendages Wise is excluded from the epithelial signaling centers where Lrp4 is expressed (Laurikkala et al., 2003; Weatherbee et al., 2006). During mammary placode formation, Lrp4 was expressed in the placodal epithelial cells similar to Lef-1 while Wise expression was strong in the surrounding epithelial and mesenchymal cells (FIGS. 5A and 5A′). Comparison of TopGal, which marks the epithelial signaling centers, and the Wise-LacZ reporter further demonstrates that the complementary expression pattern of Lrp4 and Wise is a common feature of skin appendage formation (FIG. 11).

In Wise-null females, changes in the position and number of nipples were observed (FIG. 5B). In control females, there was only a modest level of variation in the distance between nipples #2 and 3 (Data not shown). However, in the majority of Wise-null females, the distance was greatly reduced, and with a low frequency (4/18), the two nipples were fused or juxtaposed next to each other. In addition, Wise-null females frequently displayed supernumerary nipples around normal ones. Next, changes in TopGal expression in Wise-null mice were examined (FIGS. 5C-5D). In the mutants at E12.0, the placodes appeared modestly enlarged, but formed at the normal positions with no clear sign of delay in placode assembly. However, by E12.5, mutant placodes were further enlarged with an increased number of TopGal-expressing cells. Some of these TopGal-expressing cells were observed outside the placodes, in particular in the region between placodes #2 and 3, and formed a bridge connecting the two placodes. Histological sections revealed that the expanded TopGal expression was associated with abnormal morphology of the mammary epithelium in the mutant. The distance between the two placodes/buds became gradually reduced in most mutants often leading to fusion by E14.5 (4/12), consistent with the adult nipple phenotypes. Similar to the observation in Lrp4^{mdig/mdig} mice, this abnormal spacing between the placodes was associated with reduced proliferation in the interplacodal region (FIG. 11). These data indicate that Wise and Lrp4 have a similar role in controlling the distribution and number of the mammary epithelial cells, but Wise is largely dispensable for placode initiation.

In addition to the mammary defects, Wise-null mice displayed supernumerary vibrissal follicles with a frequency lower than that of Lrp4^{mitt/mitt} mice (FIG. 11; data not shown). Overall, the data suggest that Lrp4 and Wise are required for common processes in skin appendage development, but Lrp4 has additional roles. Narhi et al independently reported similar defects in mammary glands and vibrissae of Wise-null mice (Narhi et al., 2012). Relatively milder mammary defects, such as lack of fusion described by Narhi et al., are probably due to differences in strain background.

### Example 9

### Reduction in the Dosage of Lrp5 and B-Catenin Ameliorates the Wise-Null Mammary Phenotypes

Whether changes in Wnt/β-catenin signaling account for the Wise-null mammary defects were tested genetically. Wise-null mice were crossed with Lrp5 mutants to generate double homozygous mutants. It was found that removing both copies of Lrp5 significantly rescues the abnormal spacing and ectopic TopGal expression of Wise-null mammary buds (FIGS. 5E-5F). In addition, epithelial inactivation of β-catenin eliminated the ectopic TopGal expression around the buds and restored the normal spacing between the buds #2 and 3 in Wise-null mice (FIGS. 5E-5F). Together, these genetic interactions suggest that elevated Wnt/β-catenin signaling is the primary cause of mammary defects in Wise-null mice.

### Example 10

### Over-Expression of Wise Reduces the Number of Mammary Epithelial Cells

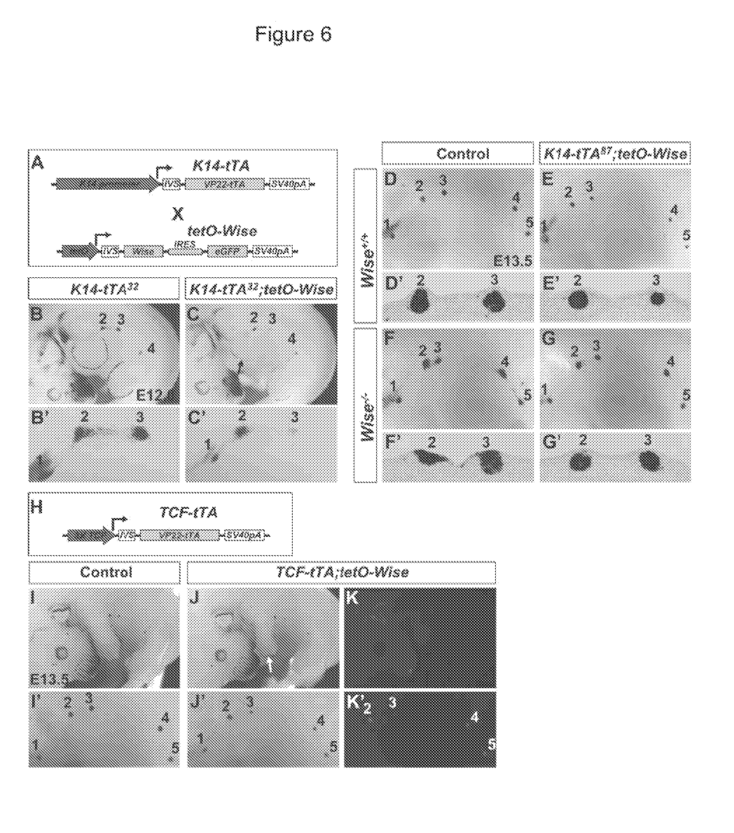

To complement and validate predicted roles for Wise based on loss-of-function analyses, whether over-expression of Wise using the Keratin 14 promoter (K14-Wise) (Ahn et al., 2010) can reduce the number of placodal epithelial cells was investigated. K14-Wise embryos showed defects in development of hair/vibrissal follicles, mammary placodes and limbs with reduced TopGal expression (FIG. 12). Due to the challenge in maintaining viable K14-Wise mice, the inventors developed a bi-transgenic Tet-off system in which expression of tetracycline-controlled transactivator (tTA) is driven by the Keratin 14 promoter (K14-tTA) in a driver line and tTA activates expression of Wise together with eGFP in an expressor line (tetO-Wise) (FIGS. 6A and 6H) (Gossen and Bujard, 1992).

Using a strong (#32) or a moderate (#87) K14-tTA driver, the mammary defects of the K14-Wise embryos were reproduced (FIGS. 6B-6E). Wise over-expression led to a significant reduction in the number of mammary epithelial cells present around the placodes (FIGS. 6B′-6C′). Importantly, Wise overexpression in the epithelium was sufficient to restore the normal morphology and spacing of the placodes in Wise-null mice (FIGS. 6D-6G′). Using the promoter with multiple TCF binding sites, similar mammary defects were observed even when Wise was over-expressed specifically in the placodes (FIGS. 6I-6K and 6I′-6K′). Because Wise is not normally expressed in the placodes, these gain-of-function phenotypes are consistent with the non-cell-autonomous function of Wise as a secreted protein. Together, the loss- and gain-of-function analyses suggest that Wise controls the number and distribution of the mammary epithelial cells during placode formation by inhibiting Wnt/β-catenin signaling.

### Example 11

### Wise Requires Lrp4 to Exert its Function In Vivo

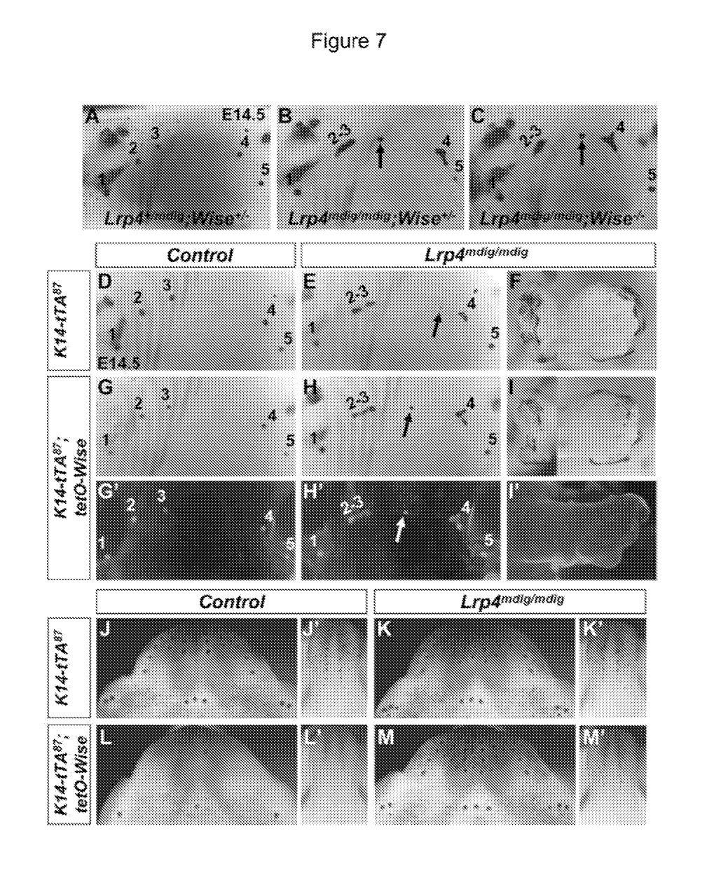

The overall similarities in skin defects and elevated Wnt signaling in Lrp4 and Wise mutants raise the question of whether Lrp4 and Wise act through a common or parallel pathways. To genetically test this idea, combinatorial mutants of the two genes were generated. No mammary defect was observed in transheterozygotes, and defects in double homozygous mutants were indistinguishable from those of Lrp4^{mdig/mdig} g mice during embryonic mammary development (FIGS. 7A-7C). A similar genetic interaction was observed with a null allele, Lrp4^{mitt} (Data not shown). This epistasis analysis suggests that inactivating Wise does not exacerbate the defects in Lrp4 mutants.

Considering the close genetic interaction of both genes with the components of Wnt/β-catenin pathway, this lack of synergy or additive effect between the two mutants suggests that Lrp4 and Wise may be acting on the same pathway to inhibit Wnt/β-catenin signaling and Lrp4 acts downstream of Wise. Alternatively, it is possible that Lrp4 and Wise function independent of each other, but Lrp4 has a larger role in modulating the level of signaling activity. To distinguish between these possibilities, Wise was over-expressed in Lrp4 mutants. Elevated Wise expression would rescue the Lrp4 mutant phenotypes if Wise and Lrp4 function primarily in an independent manner. However, over-expression of Wise resulted in no changes in the limb and mammary defects of Lrp4 mutants (FIGS. 7D-7I and 7G′-7I′). While Wise over-expression reduced the number of vibrissal follicles in control mice, in the absence of Lrp4 function, K14tTA;tetO-Wise mice still displayed supernumerary vibrissal follicles (FIGS. 7J-7M). Wise over-expression also disrupted TopGal expression in the tongue, consistent with the essential role of Wnt signaling in the taste papilla development (Iwatsuki et al., 2007) (FIGS. 7J′-7L′). However, in Lrp4 mutants, only minor changes in TopGal expression were observed with Wise over-expression in the tongue (FIGS. 7K′-7M′). These data suggest that in the mammary placodes and other contexts, Wise depends on Lrp4 for its function and support the idea that Lrp4 acts downstream of Wise to inhibit Wnt signaling.

The genetic analyses have revealed that Lrp4 and Wise play stage-specific roles for proper patterning and morphogenesis of the murine mammary glands and other skin appendages through their ability to modulate Wnt/β-catenin signaling. Lrp4 has an early role in facilitating placode initiation and together Lrp4 and Wise have later roles in induction and/or maintenance of precursor cells. Through loss-, gain-of-function and epistasis analyses, it was found that Wise requires Lrp4 to exert its activity. Together the data suggest a model whereby Wise and Lrp4 work in concert to modulate the activity of Wnt signaling though a common mechanism. These findings have important implications for a mechanistic understanding of how Wnt antagonists participate in the precise control of Wnt signaling to regulate cellular processes involved in ectodermal placode formation.

### Example 12

### Lrp4 and Wise Control Patterning of the Mammary Placodes

Development of mammary glands provides an opportunity to study spatiotemporal patterning of ectodermal organs since multiple placodes form along the mammary lines in a fairly well-defined order. The analyses of Lrp4 and Wise mutant mice have provided insight on the cellular processes that control the transition from stretches of thickened epithelium into precisely spaced placodes.

First, initiation of the placodes requires assembly of the precursor cells. In Lrp4 mutants, even when comparable numbers of cells were present around the site of placode formation, they were loosely assembled with a smaller degree of invagination compared with those of control mice. This delay in placode assembly suggests that Lrp4 normally facilitates aggregation of the precursor cells.

Second, the number of the precursor cells needs to be tightly controlled for proper morphogenesis of individual placodes and maintenance of spacing between them. The significant increase in the number of Wnt reporter-positive cells in Lrp4 and Wise mutants suggests that both Lrp4 and Wise have a role in limiting the mammary fate to a defined number of epithelial cells. This may be achieved by suppressing maintenance of mammary fate in existing precursor cells or by blocking induction of new precursor cells as mammary epithelial cells tend to proliferate at a very low rate.

In addition, migration of the mammary precursor cells may play an important role in placode initiation and morphogenesis. The sustained presence of the precursor cells in the interplacodal regions of Lrp4 and Wise mutants suggest that these cells fail to migrate to the normal sites of placode formation. These ectopic precursor cells then interfere with morphogenesis of normal placodes and give rise to supernumerary placodes. The extent of migration along the mammary line is not well characterized. It is possible that cell movement is limited to cells near the sites of placode formation and cells farther away from the placodes lose their potential to become mammary epithelial cells.

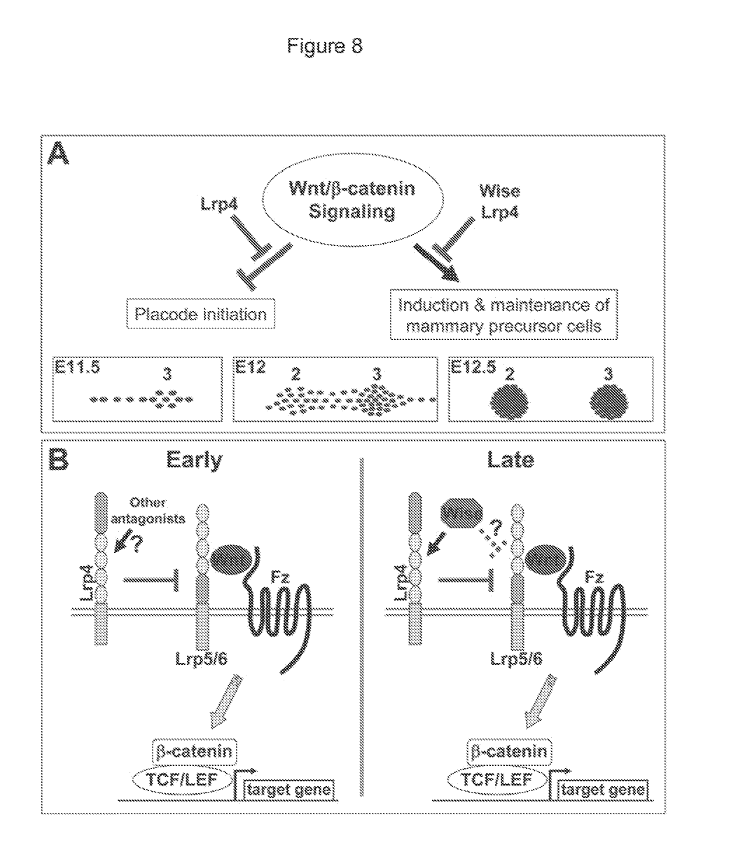

Disruption in any of the above processes would lead to defects in the number, morphogenesis and position of the mammary placodes. Mutant phenotypes suggest that initially Lrp4 is predominantly required for assembly of the placodes, and later both Lrp4 and Wise play a role in the number of the precursor cells (FIG. 8).

### Example 13

### Lrp4 and Wise are Required for Development of Other Skin Appendages

Consistent with the idea that the molecular mechanisms for early morphogenesis are shared among the skin appendages, both Lrp4 and Wise mutants display similar abnormalities in patterning of hair and vibrissal follicles with stronger defects observed in Lrp4 mutants. Interestingly, the formation of supernumerary vibrissal follicles is preceded by delayed placode morphogenesis with a broader distribution of the Wnt-active precursor cells in Lrp4 mutants. A delay in placode formation was also observed in the primary hair follicles of Lrp4 mutants. These delays are reminiscent of the defects observed during the mammary placode formation. Focalization of the epithelial precursor cells and associated Wnt activity are commonly seen during the formation of the skin placodes as well as AER (Mikkola and Millar, 2006; Fernandez-Teran and Ros, 2008). It is possible that Lrp4 and Wise modulate Wnt signaling in those precursor cells to control cellular processes such as cell movement, cell shape change, cell-cell adhesion and cell proliferation, which are important for patterning and morphogenesis of the skin placodes (Jamora et al., 2003).

### Example 14

### Lrp4 and Wise Inhibit Wnt/B-Catenin Pathway During Mammary Development

The data showed that the mammary defects of Lrp4 and Wise mutants can be rescued by reducing the dose of Lrp5/6 and β-catenin. This genetic interaction indicates that elevated Wnt/β-catenin signaling is responsible for the mammary defects and suggests that Lrp4 and Wise directly antagonize Wnt/β-catenin signaling instead of acting indirectly via another signaling pathway. This is consistent with the previous studies which provided genetic evidence that Wise functions as a Wnt inhibitor in tooth development (Munne et al., 2009; Ahn et al., 2010).

The genetic analyses also demonstrate that Wnt/β-catenin signaling is essential for induction and/or maintenance of mammary precursor cells, but its activity needs to be tightly controlled to achieve a proper number of these cells (FIG. 8A). Early inhibition of Wnt/β-catenin signaling would lead to loss or reduction of the precursor cells disrupting placode formation as seen in K14-Dkk1 (Chu et al., 2004) and K14-Wise mice. Conversely, elevated Wnt/β-catenin signaling in Lrp4 and Wise mutants results in increase in the number of mammary epithelial cells. Another important implication of our study is that a temporal reduction of Wnt/β-catenin signaling is necessary to facilitate initiation of mammary placodes and this seems to be applicable to other skin appendages. This provides additional insight into the diverse roles played by Wnt signaling throughout placode development and underscores the importance of Wnt inhibitory function of Lrp4 and Wise in these processes.

### Example 15

### Wise Requires Lrp4 to Modulate Wnt/B-Catenin Signaling

Similar to other LDL receptor-related proteins, Lrp4 is implicated in regulating different signaling pathways (May et al., 2007; Willnow et al., 2007). With its multiple ligand binding motifs in the extracellular domain, Lrp4 has the ability to bind in vitro to secreted Wnt and Bmp antagonists (Ohazama et al., 2008; Choi et al., 2009). Interestingly, in both humans and mice Lrp4 mutations phenocopy defects caused by deficiency of individual Wnt antagonists in a tissue-specific manner. For example, limb defects of Lrp4 mutants are similar to those of Dkk1 mutant mice (MacDonald et al., 2004), and bone overgrowth of human patients with LRP4 mutations is reminiscent of bone defects caused by SOST and Dkk1 mutations (Balemans et al., 2001; Morvan et al., 2006). Lastly, Lrp4 and Wise mutant mice share defects in tooth, mammary glands and other skin appendages (Ohazama et al., 2008; this study). These observations imply that interplay between Lrp4 and the Wnt antagonists may play an important role in modulating Wnt/β-catenin signaling in many developmental and physiological contexts.

While genetic evidence for such interplay has been lacking, the observation that Wise gain-of-function phenotypes depend on Lrp4 in various tissue contexts provides important insight on this issue. Based on loss- and gain-of-function analyses in this study, without being bound by a particular theory, it is believed that Lrp4 and Wise act through a common mechanism where Lrp4 lies downstream of Wise in the pathway leading to inhibition of Wnt/β-catenin signaling (FIG. 8B). In this model, Lrp4 is required to mediate or potentiate the Wnt inhibitory function of Wise and possibly other Wnt antagonists. This model is consistent with the lack of synergy or additive effects between Lrp4 and Wise mutants in our epistasis analyses of mammary and vibrissae phenotypes of Lrp4 and Wise mutants (FIG. 7 and data not shown). The earlier Wise-independent role for Lrp4 and the relatively milder mammary defects of Wise-null mice might be attributed to function of Lrp4 alone or compensation by other antagonists.

### DOCUMENTS

Ahn, Y., Sanderson, B. W., Klein, O. D. and Krumlauf, R. (2010) ‘Inhibition of Wnt signaling by Wise (Sostdc1) and negative feedback from Shh controls tooth number and patterning’, Development 137(19): 3221-31.
Balemans, W., Ebeling, M., Patel, N., Van Hul, E., Olson, P., Dioszegi, M., Lacza, C., Wuyts, W., Van Den Ende, J., Willems, P. et al. (2001) ‘Increased bone density in sclerosteosis is due to the deficiency of a novel secreted protein (SOST)’, Human Molecular Genetics 10(5): 537-43.
Balinsky, B. I. (1950) ‘On the prenatal growth of the mammary gland rudiment in the mouse’, Journal of Anatomy 84(3): 227-35.
Boras-Granic, K., Chang, H., Grosschedl, R. and Hamel, P. A. (2006) ‘Lef1 is required for the transition of Wnt signaling from mesenchymal to epithelial cells in the mouse embryonic mammary gland’, Developmental Biology 295(1): 219-31.
Brault, V., Moore, R., Kutsch, S., Ishibashi, M., Rowitch, D. H., McMahon, A. P., Sommer, L., Boussadia, O. and Kemler, R. (2001) ‘Inactivation of the beta-catenin gene by Wnt1-Cre-mediated deletion results in dramatic brain malformation and failure of craniofacial development’, Development 128(8): 1253-64.
Choi, H. Y., Dieckmann, M., Herz, J. and Niemeier, A. (2009) ‘Lrp4, a novel receptor for Dickkopf 1 and sclerostin, is expressed by osteoblasts and regulates bone growth and turnover in vivo’, PLoS One 4(11): e7930.
Chu, E. Y., Hens, J., Andl, T., Kairo, A., Yamaguchi, T. P., Brisken, C., Glick, A., Wysolmerski, J. J. and Millar, S. E. (2004) ‘Canonical WNT signaling promotes mammary placode development and is essential for initiation of mammary gland morphogenesis’, Development 131(19): 4819-29.
Cowin, P. and Wysolmerski, J. (2010) ‘Molecular mechanisms guiding embryonic mammary gland development’, Cold Spring Harb Perspect Biol 2(6): a003251.
DasGupta, R. and Fuchs, E. (1999) ‘Multiple roles for activated LEF/TCF transcription complexes during hair follicle development and differentiation’, Development 126(20): 4557-68.
Dassule, H. R., Lewis, P., Bei, M., Maas, R. and McMahon, A. P. (2000) ‘Sonic hedgehog regulates growth and morphogenesis of the tooth’, Development 127(22): 4775-85.
Dietrich, M. F., van der Weyden, L., Prosser, H. M., Bradley, A., Herz, J. and Adams, D. J. (2010) ‘Ectodomains of the LDL receptor-related proteins LRP1b and LRP4 have anchorage independent functions in vivo’, PLoS One 5(4): e9960.
Duchesne, A., Gautier, M., Chadi, S., Grohs, C., Floriot, S., Gallard, Y., Caste, G., Ducos, A. and Eggen, A. (2006) ‘Identification of a doublet missense substitution in the bovine LRP4 gene as a candidate causal mutation for syndactyly in Holstein cattle’, Genomics 88(5): 610-21.
Ferrer-Vaquer, A., Piliszek, A., Tian, G., Aho, R. J., Dufort, D. and Hadjantonakis, A. K. (2010). A sensitive and bright single-cell resolution live imaging reporter of Wnt/β-catenin signaling in the mouse. BMC Dev. Biol. 10, 121.

Fernandez-Teran, M. and Ros, M. A. (2008) ‘The Apical Ectodermal Ridge: morphological aspects and signaling pathways’, International Journal of Developmental Biology 52(7): 857-71.
Fliniaux, I., Mikkola, M. L., Lefebvre, S. and Thesleff, I. (2008) ‘Identification of dkk4 as a target of Eda-A1/Edar pathway reveals an unexpected role of ectodysplasin as inhibitor of Wnt signalling in ectodermal placodes’, Developmental Biology 320(1): 60-71.
Gossen, M. and Bujard, H. (1992) ‘Tight control of gene expression in mammalian cells by tetracycline-responsive promoters’, Proceedings of the National Academy of Sciences of the United States of America 89(12): 5547-51.
He, J., Ma, L., Kim, S., Nakai, J. and Yu, C. R. (2008) ‘Encoding gender and individual information in the mouse vomeronasal organ’, Science 320(5875): 535-8.
Herz, J. and Bock, H. H. (2002) ‘Lipoprotein receptors in the nervous system’, Annual Review of Biochemistry 71: 405-34.
Holmen, S. L., Giambernardi, T. A., Zylstra, C. R., Buckner-Berghuis, B. D., Resau, J. H., Hess, J. F., Glatt, V., Bouxsein, M. L., Ai, M., Warman, M. L. et al. (2004) ‘Decreased BMD and limb deformities in mice carrying mutations in both Lrp5 and Lrp6’, Journal of Bone and Mineral Research 19: 2033-40.
Howard, B. and Ashworth, A. (2006) ‘Signalling pathways implicated in early mammary gland morphogenesis and breast cancer’, PLoS Genetics 2(8): e112.

Itasaki, N., Jones, C. M., Mercurio, S., Rowe, A., Domingos, P. M., Smith, J. C. and Krumlauf, R. (2003) ‘Wise, a context-dependent activator and inhibitor of Wnt signalling’, Development 130(18): 4295-305.
Iwatsuki, K., Liu, H. X., Gronder, A., Singer, M. A., Lane, T. F., Grosschedl, R., Mistretta, C. M. and Margolskee, R. F. (2007) ‘Wnt signaling interacts with Shh to regulate taste papilla development’, Proceedings of the National Academy of Sciences of the United States of America 104(7): 2253-8.
Jamora, C., DasGupta, R., Kocieniewski, P. and Fuchs, E. (2003) ‘Links between signal transduction, transcription and adhesion in epithelial bud development’, Nature 422(6929): 317-22.
Johnson, E. B., Hammer, R. E. and Herz, J. (2005) ‘Abnormal development of the apical ectodermal ridge and polysyndactyly in Megf7-deficient mice’, Human Molecular Genetics 14(22): 3523-38.
Johnson, E. B., Steffen, D. J., Lynch, K. W. and Herz, J. (2006) ‘Defective splicing of Megf7/Lrp4, a regulator of distal limb development, in autosomal recessive mulefoot disease’, Genomics 88(5): 600-9.
Karner, C. M., Dietrich, M. F., Johnson, E. B., Kappesser, N., Tennert, C., Percin, F., Wollnik, B., Carroll, T. J. and Herz, J. (2010) ‘Lrp4 regulates initiation of ureteric budding and is crucial for kidney formation—a mouse model for Cenani-Lenz syndrome’, PLoS One 5(4): e10418.
Kato, M., Patel, M. S., Levasseur, R., Lobov, I., Chang, B. H., Glass, D. A., 2nd, Hartmann, C., Li, L., Hwang, T. H., Brayton, C. F. et al. (2002) ‘Cbfa1-independent decrease in osteoblast proliferation, osteopenia, and persistent embryonic eye vascularization in mice deficient in Lrp5, a Wnt coreceptor’, Journal of Cell Biology 157(2): 303-14.
Kim, N., Stiegler, A. L., Cameron, T. O., Hallock, P. T., Gomez, A. M., Huang, J. H., Hubbard, S. R., Dustin, M. L. and Burden, S. J. (2008) ‘Lrp4 is a receptor for Agrin and forms a complex with MuSK’, Cell 135(2): 334-42.
Krishnan, V., Bryant, H. U., MacDougald, O. A. (2006) ‘Regulation of bone mass by Wnt signaling’ J Clin Invest. 116(5):1202-1209.
Laurikkala, J., Kassai, Y., Pakkasjarvi, L., Thesleff, I. and Itoh, N. (2003) ‘Identification of a secreted BMP antagonist, ectodin, integrating BMP, FGF, and SHH signals from the tooth enamel knot’, Developmental Biology 264(1): 91-105.
Lee, E. C., Yu, D., Martinez de Velasco, J., Tessarollo, L., Swing, D. A., Court, D. L., Jenkins, N. A. and Copeland, N. G. (2001) ‘A highly efficient Escherichia coli-based chromosome engineering system adapted for recombinogenic targeting and subcloning of BAC DNA’, Genomics 73(1): 56-65.
Lee, M. Y., Racine, V., Jagadpramana, P., Sun, L., Yu, W., Du, T., Spencer-Dene, B., Rubin, N., Le, L., Ndiaye, D. et al. (2011) ‘Ectodermal Influx and Cell Hypertrophy Provide Early Growth for All Murine Mammary Rudiments, and Are Differentially Regulated among Them by Gli3’, PLoS One 6(10): e26242.
Leupin, O., Piters, E., Halleux, C., Hu, S., Kramer, I., Morvan, F., Bouwmeester, T., Schirle, M., Bueno-Lozano, M., Fuentes, F. J. et al. (2011) ‘Bone overgrowth-associated mutations in the LRP4 gene impair sclerostin facilitator function’, Journal of Biological Chemistry 286(22): 19489-500.
Li, X., Zhang, Y., Kang, H., Liu, W., Liu, P., Zhang, J., Harris, S. E. and Wu, D. (2005) ‘Sclerostin binds to LRP5/6 and antagonizes canonical Wnt signaling’, Journal of Biological Chemistry 280(20): 19883-7.
Li, Y., Pawlik, B., Elcioglu, N., Aglan, M., Kayserili, H., Yigit, G., Percin, F., Goodman, F., Nurnberg, G., Cenani, A. et al. (2010) ‘LRP4 Mutations Alter Wnt/□-Catenin Signaling and Cause Limb and Kidney Malformations in Cenani-Lenz Syndrome’, American Journal of Human Genetics 86(5): 696-706.
Lindvall, C., Evans, N. C., Zylstra, C. R., Li, Y., Alexander, C. M. and Williams, B. O. (2006) ‘The Wnt signaling receptor Lrp5 is required for mammary ductal stem cell activity and Wnt1-induced tumorigenesis’, Journal of Biological Chemistry 281(46): 35081-7.
Lindvall, C., Zylstra, C. R., Evans, N., West, R. A., Dykema, K., Furge, K. A. and Williams, B. O. (2009) ‘The Wnt co-receptor Lrp6 is required for normal mouse mammary gland development’, PLoS One 4(6): e5813.
Lintern, K. B., Guidato, S., Rowe, A., Saldanha, J. W. and Itasaki, N. (2009) ‘Characterization of wise protein and its molecular mechanism to interact with both Wnt and BMP signals’, Journal of Biological Chemistry 284(34): 23159-68.
MacDonald, B. T., Adamska, M. and Meisler, M. H. (2004) ‘Hypomorphic expression of Dkk1 in the doubleridge mouse: dose dependence and compensatory interactions with Lrp6’, Development 131(11): 2543-52.

MacDonald, B. T., Tamai, K. and He, X. (2009) ‘Wnt/beta-catenin signaling: components, mechanisms, and diseases’, Developmental Cell 17(1): 9-26.
May, P., Woldt, E., Matz, R. L. and Boucher, P. (2007) ‘The LDL receptor-related protein (LRP) family: an old family of proteins with new physiological functions’, Annals of Medicine 39(3): 219-28.
Mikkola, M. L. and Millar, S. E. (2006) ‘The mammary bud as a skin appendage: unique and shared aspects of development’, Journal of Mammary Gland Biology and Neoplasia 11(3-4): 187-203.
Milenkovic, L., Goodrich, L. V., Higgins, K. M. and Scott, M. P. (1999) ‘Mouse patched1 controls body size determination and limb patterning’, Development 126(20): 4431-40.
Morvan, F., Boulukos, K., Clement-Lacroix, P., Roman Roman, S., Suc-Royer, I., Vayssiere, B., Ammann, P., Martin, P., Pinho, S., Pognonec, P. et al. (2006) ‘Deletion of a single allele of the Dkk1 gene leads to an increase in bone formation and bone mass’, Journal of Bone and Mineral Research 21(6): 934-45.
Munne, P. M., Tummers, M., Jarvinen, E., Thesleff, I. and Jernvall, J. (2009) ‘Tinkering with the inductive mesenchyme: Sostdc1 uncovers the role of dental mesenchyme in limiting tooth induction’, Development 136(3): 393-402.
Narhi, K., Tummers, M., Ahtiainen, L., Itoh, N., Thesleff, I. and Mikkola, M. L. (2012) ‘Sostdc1 defines the size and number of skin appendage placodes’, Developmental Biology 364(2): 149-61.

Ohazama, A., Johnson, E. B., Ota, M. S., Choi, H. J., Porntaveetus, T., Oommen, S., Itoh, N., Eto, K., Gritli-Linde, A., Herz, J. et al. (2008) ‘Lrp4 modulates extracellular integration of cell signaling pathways in development’, PLoS One 3(12): e4092.
Pinson, K. I., Brennan, J., Monkley, S., Avery, B. J. and Skarnes, W. C. (2000) ‘An LDL-receptor-related protein mediates Wnt signalling in mice.’, Nature 407: 535-538.
Pispa, J. and Thesleff, I. (2003) ‘Mechanisms of ectodermal organogenesis’, Developmental Biology 262(2): 195-205.
Propper, A. Y. (1978) ‘Wandering epithelial cells in the rabbit embryo milk line. A preliminary scanning electron microscope study’, Developmental Biology 67(1): 225-31.
Robinson, G. W. (2007) ‘Cooperation of signalling pathways in embryonic mammary gland development’, Nature Reviews Genetics 8(12): 963-72.
Schmidt-Ullrich, R. and Paus, R. (2005) ‘Molecular principles of hair follicle induction and morphogenesis’, BioEssays 27(3): 247-61.
Semenov, M., Tamai, K. and He, X. (2005) ‘SOST is a ligand for LRP5/LRP6 and a Wnt signaling inhibitor’, Journal of Biological Chemistry 280(29): 26770-5.
Semenov, M. V., Tamai, K., Brott, B. K., Kuhl, M., Sokol, S. and He, X. (2001) ‘Head inducer Dickkopf-1 is a ligand for Wnt coreceptor LRP6’, Current Biology 11(12): 951-61.
Simon-Chazottes, D., Tutois, S., Kuehn, M., Evans, M., Bourgade, F., Cook, S., Davisson, M. T. and Guenet, J. L. (2006) ‘Mutations in the gene encoding the low-density lipoprotein receptor LRP4 cause abnormal limb development in the mouse’, Genomics 87(5): 673-7.
Soriano, P. (1999) ‘Generalized lacZ expression with the ROSA26 Cre reporter strain’, Nature Genetics 21(1): 70-1.
van Genderen, C., Okamura, R. M., Farinas, I., Quo, R. G., Parslow, T. G., Bruhn, L. and Grosschedl, R. (1994) ‘Development of several organs that require inductive epithelial-mesenchymal interactions is impaired in LEF-1-deficient mice’, Genes & Development 8(22): 2691-703.
Veltmaat, J. M., Van Veelen, W., Thiery, J. P. and Bellusci, S. (2004) ‘Identification of the mammary line in mouse by Wnt10b expression’, Developmental Dynamics 229(2): 349-56.
Weatherbee, S. D., Anderson, K. V. and Niswander, L. A. (2006) ‘LDL-receptor-related protein 4 is crucial for formation of the neuromuscular junction’, Development 133(24): 4993-5000.
Willnow, T. E., Hammes, A. and Eaton, S. (2007) ‘Lipoproteins and their receptors in embryonic development: more than cholesterol clearance’, Development 134(18): 3239-49.
Yamakado, M. and Yohro, T. (1979) ‘Subdivision of mouse vibrissae on an embryological basis, with descriptions of variations in the number and arrangement of sinus hairs and cortical barrels in BALB/c (nu/+; nude, nu/nu) and hairless (hr/hr) strains’, American Journal of Anatomy 155(2): 153-73.
Zhang, B., Luo, S., Wang, Q., Suzuki, T., Xiong, W. C. and Mei, L. (2008) ‘LRP4 serves as a coreceptor of agrin’, Neuron 60(2): 285-97.

Zhou, C. J., Wang, Y. Z., Yamagami, T., Zhao, T., Song, L. and Wang, K. (2010) ‘Generation of Lrp6 conditional gene-targeting mouse line for modeling and dissecting multiple birth defects/congenital anomalies’, Developmental Dynamics 239(1): 318-26.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. An antibody that modulates binding between Lrp5 and WISE or Lrp6 and WISE, but does not modulate binding between Lrp4 and WISE.

2. The antibody according to claim 1, wherein the antibody prevents binding between Lrp5 and WISE or Lrp6 and WISE.

3. The antibody according to claim 1, wherein the antibody binds specifically to Lrp5 or Lrp6.

4. The antibody according to claim 1, wherein the antibody binds specifically to Lrp5.

5. The antibody according to claim 1, wherein the antibody binds specifically to Lrp6.

6. The antibody according to claim 1, wherein the Lrp4, Lrp5, and Lrp6 are human.

7. The antibody according to claim 1, wherein the antibody is monoclonal.

8. The antibody according to claim 1, wherein the antibody is human, humanized, or chimeric.

9. The antibody according to claim 3, wherein the antibody specifically binds to Lrp5 or Lrp6 at one or more sequences selected from the group consisting of SEQ ID NOs: 1-39.

10. The antibody according to claim 9, wherein the antibody specifically binds to Lrp5 at one or more sequences selected from the group consisting of SEQ ID NOs: 1-15.

11. The antibody according to claim 9, wherein the antibody specifically binds to Lrp6 at one or more sequences selected from the group consisting of SEQ ID NOs: 1-3 and 16-27.

12. The antibody according to claim 9, wherein the antibody specifically binds to Lrp5 or Lrp6 at one or more sequences selected from the group consisting of SEQ ID NOs: 1-3 and 28-39.

13. The antibody according to claim 3, wherein the antibody specifically binds to Lrp5 or Lrp6 within a sequence selected from the group consisting of SEQ ID NOs:40-48.

14. A pharmaceutical composition comprising an antibody of claim 1 and at least one pharmaceutically acceptable excipient or diluent.

15. A method for preventing WISE binding to Lrp5 or Lrp6, but not WISE binding to Lrp4, comprising contacting Lrp5 or Lrp6 with an agent that binds to Lrp5 or Lrp6 but not Lrp4.

16. The method according to claim 15, wherein the agent is an antibody.

17. The method according to claim 16, wherein the antibody is monoclonal.

18. The method according to claim 16, wherein the antibody specifically binds to Lrp5 or Lrp6 at one or more sequences selected from the group consisting of SEQ ID NOs: 1-39.

19. The method according to claim 16, wherein antibody specifically binds to Lrp5 or Lrp6 within a sequence selected from the group consisting of SEQ ID NOs:40-48.

20. A method for preventing human WISE from binding to human Lrp5 or human Lrp6 comprising contacting human WISE with a monoclonal antibody, which antibody specifically binds to human Lrp5 or human Lrp6 but not to human Lrp4.

21. The method according to claim 20, wherein the antibody specifically binds to human Lrp5 or human Lrp6 at one or more sequences selected from the group consisting of SEQ ID NOs: 1-39.

22. The method according to claim 20, wherein the antibody specifically binds to human Lrp5 or human Lrp6 within a sequence selected from the group consisting of SEQ ID NOs:40-48.

23. A kit comprising an antibody according to claim 1.

24. A kit to modulate binding between Lrp5 and WISE or Lrp6 and WISE, but not Lrp4 and WISE comprising a pharmaceutical composition according to claim 14.
